(19) United States
(12) Patent Application Publication
(10) Pub. No.: US 2019/0287013 A1
(43) Pub. Date: Sep. 19, 2019

(54) METHODS FOR DETERMINING DOSING OF A THERAPEUTIC AGENT AND RELATED TREATMENTS

(71) Applicants: Yuan JI, Wilmette, IL (US); Juno Therapeutics, Inc., Seattle, WA (US)

(72) Inventors: He LI, Seattle, WA (US); Yuan JI, Wilmette, IL (US)

(73) Assignee: Juno Therapeutics, Inc., Seattle, WA (US)

(21) Appl. No.: 16/085,267

(22) PCT Filed: Mar. 16, 2017
(86) PCT No.: PCT/US2017/022844

§ 371 Date: Sep. 14, 2018

Related U.S. Application Data
(60) Provisional application No. 62/309,445, filed on Mar. 16, 2016.
(60) Provisional application No. 62/321,172, filed on Apr. 11, 2016.
(60) Provisional application No. 62/371,193, filed on Aug. 4, 2016.

(51) Int. Cl. G06N 7/00; G16H 20/10; G16H 50/20; G16H 10/20

(52) U.S. Cl. CPC G06N 7/005; G16H 10/20; G16H 50/20; G16H 20/10

(57) ABSTRACT

Provided herein are methods employing a toxicity and efficacy probability interval (TEPI) design for performing a clinical trial, such as a Phase I dose-finding trial. In some embodiments, the methods can be used in the dosing of subjects administered a therapy, such as adoptive cell therapy or other immunotherapy, where safety and efficacy data for a therapeutic agent can be observed in the same timeframe or period. In some embodiments, one or more of or all of the steps of the method occur at an electronic device containing one or more processors and memory, such as implemented by a computer. Also provided are methods of administering a therapeutic agent to a subject in accord with the dosing decisions.

### CROSS-REFERENCE TO RELATED APPLICATIONS

This application claims priority from U.S. provisional application No. 62/309,445 filed Mar. 16, 2016, entitled “Methods for Determining Dosing of a Therapeutic Agent and Related Treatments,” U.S. provisional application No. 62/321,172 filed Apr. 11, 2016, entitled “Methods for Determining Dosing of a Therapeutic Agent and Related Treatments,” and U.S. provisional application No. 62/371,193 filed Aug. 4, 2016, entitled “Methods for Determining Dosing of a Therapeutic Agent and Related Treatments,” the contents of each of which is incorporate by reference in its entirety.

### FIELD

The present disclosure relates to methods employing a toxicity and efficacy probability interval (TEPI) design for performing a clinical trial, such as a Phase I dose-finding trial. In some embodiments, the methods can be used in the dosing of subjects administered a therapy, such as adoptive cell therapy or other immunotherapy, where safety and efficacy data for a therapeutic agent can be observed in the same timeframe or period. In some embodiments, the TEPI design utilizes both safety data and efficacy data for the therapeutic agent in the treatment of a disease or condition to inform dose escalation decisions. In some aspects, decision rules (e.g., a dose-finding protocol) can be pre-specified prior to the start of the trial, allowing for transparency to clinicians and non-statisticians. In some embodiments, one or more of or all of the steps of the method occur at an electronic device containing one or more processors and memory, such as implemented by a computer. The present disclosure also provides methods of administering a therapeutic agent to a subject in accord with the dosing decisions.

### BACKGROUND

Various methods are available for designing clinical trials, including Phase I clinical trials. In general, in most methods for the design of Phase I clinical trials, dose escalation decisions are determined based on toxicity data only. Improved methods are needed, for example, to incorporate efficacy data into the dose escalation decision. Such methods may simplify, shorten and/or improve the robustness of the clinical trial. Provided are methods that meet such needs.

### SUMMARY

Provided in some aspects are methods for providing a dose recommendation for a therapeutic agent in a treatment course or dosing regimen. In some embodiments, the dose recommendation is for a clinical trial, such as a Phase I clinical trial. In some embodiments, by incorporating efficacy data and decisions based on efficacy data, the methods are useful for planning and/or carrying out a Phase I/II clinical trial. In some embodiments, the method is performed prior to the initiation of a clinical trial or following completion of a clinical trial.

Provided in some embodiments are methods for assessing dosing, such as dosing of a therapy or therapeutic agent. For example, in some aspects, the methods are useful for determining a dose level of a therapy or therapeutic agent, such as one to be tested, e.g., in a clinical trial.

Provided in some aspects are methods for determining an optimal dose of a therapeutic agent. For example, in some cases, the provided methods are useful for determining an optimal dose for a clinical trial, e.g., Phase II clinical trial, such as based on data from a previous clinical trial, e.g., Phase I clinical trial.

Provided in some aspects are methods for providing a dose recommendation for a therapeutic agent, such as in a clinical trial. In some embodiments, the method involves determining a joint unit probability mass (UPM) for a combination of toxicity and efficacy probability intervals in a matrix. In some embodiments, the matrix contains one or more dosing actions associated with each combination of toxicity and efficacy probability intervals. In some cases, the joint UPM is determined for one or more true or possible toxicity probabilities at current dose level i (pᵢ) and one or more true or possible efficacy probabilities at current dose level i (qᵢ). In some embodiments, the method involves identifying the combination of toxicity and efficacy intervals that has the highest joint UPM. In some instances, the method includes assigning the dosing action associated with the identified combination as a dose recommendation for each of the one or more possible toxicity and efficacy probabilities. In some embodiments, the method involves producing or outputting instructions that specify the dose recommendations.

Provided in some aspects are methods for providing a dose recommendation for a therapeutic agent, such as in a clinical trial. In some embodiments, the method involves obtaining a matrix comprising one or more dosing actions associated with a combination of toxicity and efficacy probability intervals in a clinical trial. In some cases, the method includes determining a joint unit probability mass (UPM) for each combination of toxicity and efficacy probability intervals for one or more true or possible toxicity probabilities at current dose level i (pᵢ) and one or more true or possible efficacy probabilities at current dose level i (qᵢ). In some embodiments, the method involves identifying the combination of toxicity and efficacy intervals that has the highest joint UPM. In some instances, the method includes assigning the dosing action associated with the identified combination as a dose recommendation for each of the one or more possible toxicity and efficacy probabilities. In some embodiments, the method involves producing or outputting instructions that specify the dose recommendations.

In some embodiments, the obtaining of the matrix need not involve collecting data from a subject. In some embodiments, the obtaining of the matrix need not involve estimating or calculating possible or true toxicity and efficacy probability intervals. In some embodiments, obtaining the matrix comprises receiving information about the toxicity or probability intervals, such as from a clinician or through a computer.

Provided in some aspects is a computer implemented method for providing a dose recommendation for a therapeutic agent, such as in a clinical trial. In some aspects, the method includes creating, obtaining, or receiving a matrix comprising one or more dosing actions associated with a combination of toxicity and efficacy probability intervals in a clinical trial at an electronic device having a processor and memory. In some cases, the methods involve determining, by the processor, a joint unit probability mass (UPM) for each combination of toxicity and efficacy probability intervals for one or more possible toxicity probabilities at current dose level i (pᵢ) and one or more possible efficacy probabilities at current dose level i (qᵢ) at an electronic device having a processor and memory. In some embodiments, the method involves identifying, at an electronic device having a processor and memory, the combination of toxicity and efficacy intervals that has the highest joint UPM. In some instances, the method includes assigning, at an electronic device having a processor and memory, the dosing action associated with the identified combination as a dose recommendation for each of the one or more possible toxicity and efficacy probabilities. In some embodiments, the method involves producing or outputting instructions that specify the dose recommendations at an electronic device having a processor and memory.

In some embodiments, provided are methods for providing a dose recommendation for a therapeutic agent. In some embodiments, the provided methods include: a) obtaining a matrix comprising one or more dosing actions associated with a combination of toxicity and efficacy probability intervals; b) determining a joint unit probability mass (UPM) for each combination of toxicity and efficacy probability intervals for one or more possible toxicity probabilities at current dose level i (pᵢ) and one or more possible efficacy probabilities at current dose level i (qᵢ); c) identifying the combination of toxicity and efficacy intervals that has the highest joint UPM; d) assigning the dosing action associated with the identified combination as a dose recommendation for each of the one or more possible toxicity and efficacy probabilities; and e) producing or outputting instructions that specify the dose recommendations.

In some embodiments, provided are methods for providing a dose recommendation for a therapeutic agent. In some embodiments, the provided methods include: a) identifying a combination of toxicity and efficacy intervals that has the highest joint unit probability mass (UPM) from a matrix, wherein the matrix comprises combinations of toxicity and efficacy probability intervals at current dose level i and one or more dosing actions associated with said toxicity and efficacy probabilities; b) assigning the dosing action associated with the identified combination as a dose recommendation for each of the one or more possible toxicity and efficacy probabilities; and c) producing or outputting instructions that specify the dose recommendations.

In some embodiments, also provided are computer implemented methods for providing a dose recommendation for a therapeutic agent. In some embodiments, such methods include, at an electronic device having a processor and memory: a) obtaining a matrix comprising one or more dosing actions associated with a combination of toxicity and efficacy probability intervals; b) determining, by the processor, a joint unit probability mass (UPM) for each combination of toxicity and efficacy probability intervals for one or more possible toxicity probabilities at current dose level i (pᵢ) and one or more possible efficacy probabilities at current dose level i (qᵢ); c) identifying the combination of toxicity and efficacy intervals that has the highest joint UPM; d) assigning the dosing action associated with the identified combination as a dose recommendation for each of the one or more possible toxicity and efficacy probabilities; and e) producing or outputting instructions that specify the dose recommendations.

In some embodiments, also provided are computer implemented methods for providing a dose recommendation for a therapeutic agent. In some embodiments, such methods include, at an electronic device having a processor and memory: a) identifying a combination of toxicity and efficacy intervals that has the highest joint unit probability mass (UPM) from a matrix, wherein the matrix comprises combinations of toxicity and efficacy probability intervals at current dose level i and one or more dosing actions associated with said toxicity and efficacy probabilities; b) assigning the dosing action associated with the identified combination as a dose recommendation for each of the one or more possible toxicity and efficacy probabilities; and c) producing or outputting instructions that specify the dose recommendations.

In some embodiments of the methods provided herein, the therapeutic agent is administered in a clinical trial.

In some embodiments, the matrix is created by designating two or more toxicity probability intervals of the therapeutic agent and two or more efficacy probability intervals of the therapeutic agent and assigning a dosing action to each combination of toxicity and efficacy probability intervals. In some embodiments, the matrix comprises two toxicity probability intervals. In some cases, the matrix comprises three toxicity probability intervals. In some instances, the matrix comprises four toxicity probability intervals. In some embodiments, the matrix comprises two efficacy probability intervals. In some aspects, the matrix comprises three efficacy probability intervals. In some aspects, the matrix comprises four efficacy probability intervals.

In some embodiments, the dosing action and/or dose recommendation is relative to a current dose i. In some embodiments, the dosing action and/or dose recommendation is escalate (E) to dose level i+1. In some embodiments, the dosing action and/or dose recommendation is stay (S) at dose level i. In some aspects, the dosing action and/or dose recommendation is de-escalate (D) to dose level i−1.

In some embodiments, the method further includes altering the dose recommendation, such as before producing or outputting the instructions. For example, the dose recommendation may be altered to de-escalate and do not return to the current dose if the probability that pᵢ is greater than the maximum acceptable toxicity probability (p_{T}) exceeds 0.95. In some aspects, the dose recommendation is altered to de-escalate and do not return to the current dose if the probability that qᵢ is less than the minimum acceptable efficacy probability (q_{E}) exceeds 0.7. In some cases, the dose recommendation is altered to escalate and do not return to the current dose if the probability that qᵢ is less than q_{E} exceeds 0.7.

In some embodiments, the method further includes altering the dose recommendation, such as before producing or outputting the instructions. For example, the dose recommendation may be altered to de-escalate and do not return to the current dose if pᵢ is greater than p_{T}. In some cases, the dose recommendation is altered to de-escalate and do not return to the current dose if qᵢ is less than q_{E}. In some instances, the dose recommendation is altered to escalate and do not return to the current dose if qᵢ is less than q_{E}.

In some embodiments of the methods provided herein, prior to the step of producing or outputting instructions, the method further comprises altering the dose recommendation to: (a) de-escalate and not return to current dose if pᵢ is greater than p_{T}; (b) de-escalate and not return to current dose if qᵢ is less than q_{E}; or (c) escalate and not return to current dose if qᵢ is less than q_{E}.

In some embodiments, the dose recommendation is de-escalate and do not return to the current dose level or any higher dose level, for example in some cases, due to unacceptable toxicity (denoted interchangeably as either DU or DU_{T}). In some aspects, the dose recommendation is de-escalate and do not return to the current dose level or any higher dose level, for example in some cases, due to unacceptable low efficacy (denoted interchangeably as either DEU or DU_{E}). In some cases, the dose recommendation is escalate and do not return to the current dose level or any lower dose level, for example in some cases, due to unacceptable low efficacy (denoted interchangeably as either EEU or EU).

In some embodiments, prior to obtaining the matrix, the methods involve obtaining the maximum acceptable toxicity probability (p_{T}) and minimum acceptable efficacy probability (q_{E}) of the therapeutic agent.

In some embodiments, each toxicity probability interval is defined by a start value a and an end value b and each efficacy probability interval is defined by a start value c and an end value d. In some aspects, each combination of toxicity and efficacy probability intervals is defined as (a, b)×(c, d).

In some embodiments, the matrix is, contains, or is displayed in a two-way grid (e.g., preset table). In some aspects, the one or more dosing actions associated with the combination of toxicity and efficacy probability intervals are displayed in the two-way grid (e.g., preset table).

In some embodiments, determining the joint UPM for each combination of toxicity and probability intervals involves determining the probability that pᵢ and qᵢ are contained within the combination of toxicity and probability intervals and dividing by the product of the toxicity probability interval length and the efficacy probability interval length. In some aspects, the joint UPM (JUPM) is determined by formula (1):

$$JUPM \begin{matrix}( c , d ) \\ ( a , b )\end{matrix} ≡ \frac{Pr ( {p}_{i} ∈ ( a , b ) , {q}_{i} ∈ ( c , d ) \vert 𝒟 )}{( b - a ) \times ( d - c )} , \\ 0 < a < b < 1 , 0 < c < d < 1. \qquad ( 1 )$$

In some embodiments, determining the joint UPM is based on the posterior distributions of pᵢ and qᵢ, according to Bayes' rule.

In some embodiments, the toxicity and efficacy probability intervals are associated with a rate of a toxic outcome or a rate of a response outcome, respectively. In some aspects, pᵢ is a ratio of a number of subjects experiencing a toxic outcome (xᵢ) to a total number of subjects (nᵢ), and qᵢ is a ratio of the number of subjects experiencing a response outcome (yᵢ) to the total number of subjects (nᵢ).

In some cases, the toxicity outcome is a dose-limiting toxicity (DLT). In some instances, the response outcome is a complete response (CR). In some embodiments, the response outcome is the presence of or a level of a biomarker.

In some embodiments, the instructions contain or are displayed in one or more decision tables, such as a dose recommendation decision table. In some embodiments, dose recommendations are provided for one or more possible combinations of xᵢ and yᵢ among m subjects. In some aspects, dose recommendations are provided for all possible combinations of xᵢ and yᵢ among m subjects. In some cases, m is within a range from about 1 to about 100, about 3 to about 60, or about 6 to about 30.

In some embodiments, the clinical trial is a Phase I clinical trial. In some aspects, the clinical trial is a Phase I/II clinical trial.

In some embodiments, the method is a computer implemented method, and one or more steps of the method occur at an electronic device comprising one or more processors and memory.

In some embodiments, the method is a computer implemented method, and wherein one or more of steps of obtaining a matrix, determining a joint unit probability mass (UPM) for each combination of toxicity and efficacy probability intervals, identifying the combination with the highest joint UPM, assigning the dosing action associated with the identified combination, and producing or outputting instructions occur at an electronic device comprising one or more processors and memory.

Provided in some embodiments is a computer system comprising a processor and memory, the memory comprising instructions operable to cause the processor to carry out one or more of steps of the method.

Provided in some embodiments is a computer system comprising a processor and memory, the memory comprising instructions operable to cause the processor to carry out one or more of steps of the method of any of claims 1-40, wherein the steps are selected from obtaining a matrix, determining a joint unit probability mass (UPM) for each combination of toxicity and efficacy probability intervals, identifying the combination with the highest joint UPM, assigning the dosing action associated with the identified combination, and producing or outputting instructions.

Provided in some aspects are dose recommendation instructions for performing a clinical trial produced or outputted by any of the provided methods.

Provided in some embodiments is a method of dosing a subject, such as for treating a disease or condition in the subject, such as in a clinical trial. In some embodiments, the disease or condition is a tumor or a cancer. In some embodiments, the disease or condition is a leukemia or lymphoma. In some embodiments, the disease or condition is acute lymphoblastic leukemia. In some embodiments, the disease or condition is a non-Hodgkin lymphoma (NHL).

In some aspects, the method involves selecting a dose recommendation for administering a therapeutic agent to a subject, such as a subject who has a disease or condition, based on the instructions produced or outputted by any of the methods described herein. In some cases, the dose recommendation is selected for a given combination of a number of subjects experiencing a toxic outcome (xᵢ) and a number of subjects experiencing a response outcome (yᵢ) for a total number of subjects previously treated with the therapeutic agent in the clinical trial at a current dose level i (nᵢ). In some cases, the method further includes administering the therapeutic agent to the subject at a dose level in accord with the selected dose recommendation.

Provided in some aspects is a method of dosing a subject with a therapeutic agent in a clinical trial. In some embodiments, the method involves obtaining instructions that specify dose recommendations. In some cases, the instructions were prepared by designating two or more toxicity probability intervals of a therapeutic agent and two or more efficacy probability intervals of the therapeutic agent. In some embodiments, the instructions were prepared by assigning a dosing action to each combination of toxicity and efficacy probability intervals. In some cases, the instructions were prepared by determining the joint unit probability mass (UPM) for each combination of toxicity and efficacy probability intervals for one or more true or possible toxicity probabilities at current dose level i (pᵢ) and one or more true or possible efficacy probabilities at current dose level i (qᵢ). In some embodiments, the instructions were prepared by identifying the combination of toxicity and efficacy intervals that has the highest joint UPM. In some aspects, the instructions were prepared by assigning the dosing action associated with the identified combination as a dose recommendation for each of the one or more true or possible toxicity and efficacy probabilities.

In some embodiments, the method includes selecting a dose recommendation for administering a therapeutic agent to a subject based on the instructions for a given combination of a number of subjects experiencing a toxic outcome (xᵢ) and a number of subjects experiencing a response outcome (yᵢ) for a total number of subjects previously treated with the therapeutic agent in the clinical trial at a current dose level i (nᵢ). In some cases, the method includes administering the therapeutic agent to the subject at a dose level according to the selected dose recommendation.

Provided in some aspects are methods of dosing a subject for treating a disease or condition. In some embodiments, the methods include: a) selecting a dose recommendation for administering a therapeutic agent to a subject that has a disease or condition based on the instructions produced by any of the methods provided herein, wherein the dose recommendation is selected for a given combination of a number of subjects experiencing a toxic outcome (xᵢ) and a number of subjects experiencing a response outcome (yᵢ) for a total number of subjects previously treated with the therapeutic agent at a current dose level i (nᵢ); b) administering the therapeutic agent to the subject at a dose level in accord with the selected dose recommendation.

In some embodiments, provided are methods of dosing a subject with a therapeutic agent for treating a disease or condition. In some embodiments, the methods include: a) obtaining instructions that specify dose recommendations, wherein the instructions were produced by: i) designating two or more toxicity probability intervals of a therapeutic agent and two or more efficacy probability intervals of the therapeutic agent; ii) assigning a dosing action to each combination of toxicity and efficacy probability intervals; iii) determining the joint unit probability mass (UPM) for each combination of toxicity and efficacy probability intervals for one or more possible toxicity probabilities at current dose level i (pᵢ) and one or more possible efficacy probabilities at current dose level i (qᵢ); iv) identifying the combination of toxicity and efficacy intervals that has the highest joint UPM; and v) assigning the dosing action associated with the identified combination as a dose recommendation for each of the one or more possible toxicity and efficacy probabilities, thereby producing the instructions; b) selecting a dose recommendation for administering a therapeutic agent to a subject that has a disease or condition based on the instructions for a given combination of a number of subjects experiencing a toxic outcome (xᵢ) and a number of subjects experiencing a response outcome (yᵢ) for a total number of subjects previously treated with the therapeutic agent in the clinical trial at a current dose level i (nᵢ); and c) administering the therapeutic agent to the subject at a dose level according to the selected dose recommendation.

In some embodiments, provided are methods of dosing a subject with a therapeutic agent for treating a disease or condition. In some embodiments, the methods include: administering a therapeutic agent to a subject that has a disease or condition based at a dose level according to a selected dose recommendation selected from instructions for a given combination of a number of subjects experiencing a toxic outcome (xᵢ) and a number of subjects experiencing a response outcome (yᵢ) for a total number of subjects previously treated with the therapeutic agent at a current dose level i (nᵢ); wherein the instructions were produced by: i) designating two or more toxicity probability intervals of a therapeutic agent and two or more efficacy probability intervals of the therapeutic agent; ii) assigning a dosing action to each combination of toxicity and efficacy probability intervals; iii) determining the joint unit probability mass (UPM) for each combination of toxicity and efficacy probability intervals for one or more possible toxicity probabilities at current dose level i (pᵢ) and one or more possible efficacy probabilities at current dose level i (qᵢ); iv) identifying the combination of toxicity and efficacy intervals that has the highest joint UPM; and v) assigning the dosing action associated with the identified combination as a dose recommendation for each of the one or more possible toxicity and efficacy probabilities, thereby producing the instructions.

In some embodiments of the methods provided herein, therapeutic agent is administered for a clinical trial.

In some embodiments, the dosing action and/or dose recommendation is escalate (E) to dose level i+1. In some embodiments, the dosing action and/or dose recommendation is stay (S) at dose level i. In some embodiments, the dosing action and/or dose recommendation is de-escalate (D) to dose level i−1.

In some embodiments, prior to producing the instructions, the dose recommendation is altered to de-escalate and not return to current dose if the probability that pᵢ is greater than the maximum acceptable toxicity probability (p_{T}) exceeds 0.95. In some embodiments, prior to producing the instructions, the dose recommendation is altered to de-escalate and not return to current dose if the probability that qᵢ is less than the minimum acceptable efficacy probability (q_{E}) exceeds 0.7. In some embodiments, prior to producing the instructions, the dose recommendation is altered to escalate and not return to current dose if the probability that qᵢ is less than q_{E} exceeds 0.7.

In some embodiments, prior to producing the instructions, the dose recommendation is altered to de-escalate and not return to current dose if pᵢ is greater than p_{T}. In some embodiments, prior to producing the instructions, the dose recommendation is altered to de-escalate and not return to current dose if qᵢ is less than q_{E}. In some embodiments, prior to producing the instructions, the dose recommendation is altered to escalate and not return to current dose if qᵢ is less than q_{E}.

In some embodiments, the dose recommendation is de-escalate and do not return to the current dose level or any higher dose level, for example in some cases due to toxicity (denoted interchangeably as either DU or DU_{T}). In some embodiments, the dose recommendation is de-escalate and do not return to the current dose level or any higher dose level, for example in some cases due to low efficacy (denoted interchangeably as either DEU or DU_{E}). In some embodiments, the dose recommendation is escalate and do not return to the current dose level or any lower dose level (denoted interchangeably as either EEU or EU).

In some embodiments, prior to obtaining instructions, the maximum acceptable toxicity probability (p_{T}) and minimum acceptable efficacy probability (q_{E}) of the therapeutic agent are designated, provided, or obtained.

In some embodiments, the selected dose is determined based on the number of subjects previously treated in the clinical trial and the actual probabilities of toxic outcomes and response outcomes among the subjects previously treated. In some cases, the subject is part of a cohort of subjects and all subjects in the cohort are administered the therapeutic agent at the same dose level. In some embodiments, the method is repeated for remaining subjects in the clinical trial. In some aspects, the clinical trial in terminated when the number of subjects enrolled reaches a pre-specified maximum. In some embodiments, the pre-specified maximum is within a range from about 1 to about 100, about 3 to about 60, or about 6 to about 30. In some embodiments, the pre-specified maximum is or is about 6, 9, 12, 15, 18, 21, 24, 27, 30, 45, 75, or more.

In some cases, the method involves identifying an optimal dose level, wherein the optimal dose level is associated with the highest probability that pᵢ is less than p_{T} and qᵢ is less than q_{E}. In some aspects, the optimal dose level is identified based on a combined utility function determined from safety and efficacy utility functions. In some instances, the combined utility function is determined as:

U(p,q)=ƒ₁(p)ƒ₂(q)

In some embodiments, the optimal dose level comprises the largest combined posterior utility. In some aspects, the maximum combined posterior utility is or is determined as:

î=argmaxᵢE[U(pᵢ,qᵢ)| [symbol] ]

In some cases, the clinical trial is a Phase I clinical trial. In some aspects, the clinical trial is a Phase I/II clinical trial.

In some embodiments, the toxicity outcome is a dose-limiting toxicity (DLT). In some embodiments, the toxicity outcome is the presence or absence of one or more biomarkers or a level of one or more biomarkers.

In some embodiments, the response outcome is a complete response (CR). In some embodiments, the response outcome is the presence or absence of one or more biomarkers or a level of one or more biomarkers.

In some embodiments, the therapeutic agent is one for which the response outcome can be assessed within a timeframe in which the toxicity outcome can be assessed.

In some embodiments, the therapeutic agent is for treating a tumor or cancer. In some embodiments, the therapeutic agent is or a small molecule, a gene therapy, a transplant or an adoptive cell therapy. In some embodiments, the therapeutic agent is or comprises an adoptive cell therapy.

In some embodiments, the adoptive cell therapy comprises cells expressing a chimeric antigen receptor (CAR). In some such embodiments, the CAR expressed by the cells specifically binds to an antigen expressed by a cell or tissue of the disease or condition. In some such embodiments, the cells are administered at a dose level that is between about 0.5×10⁶ cells/kg body weight of the subject and 6×10⁶ cells/kg, between about 0.75×10⁶ cells/kg and 2.5×10⁶ cells/kg, between about 1×10⁶ cells/kg and 2×10⁶ cells/kg, between about 2×10⁶ cells per kilogram (cells/kg) body weight and about 6×10⁶ cells/kg, between about 2.5×10⁶ cells/kg and about 5.0×10⁶ cells/kg, or between about 3.0×10⁶ cells/kg and about 4.0×10⁶ cells/kg, each inclusive.

In some embodiments, the number of cells administered is between about 1×10⁶ and about 1×10⁸ CAR expressing cells, between about 2×10⁶ and about 5×10⁶ CAR expressing cells, between about 1×10⁷ and about 5×10⁷ CAR expressing cells, or between about 5×10⁷ and about 1×10⁸ total CAR expressing cells.

In some embodiments, the dose of cells is administered in a single pharmaceutical composition comprising the cells of the dose. In some embodiments, the dose is a split dose, wherein the cells of the dose are administered in a plurality of compositions, collectively comprising the cells of the dose, which, optionally, are administered over a period of no more than three days.

### BRIEF DESCRIPTION OF THE DRAWINGS

FIG. 1 shows graphical representations of safety and efficacy utility functions.

FIG. 2 shows the relationship between sample size and the probability of selecting the most desirable dose in three exemplary scenarios, either scenario 3 (Scrn 3), scenario 7 (Scrn 7) or scenario 8 (Scrn 8) as described in Examples 7, 5 or 6, respectively.

### DETAILED DESCRIPTION

### I. Dose Recommendation Based on Toxicity and Efficacy Probability Interval (TEPI) Model

Provided herein are methods for determining and/or providing a dose recommendation for a therapeutic agent based on a combination of toxicity and efficacy outcome probabilities, such as for implementing dosing decisions in a clinical trial, such as a Phase I dose-finding clinical trial. Also provided are methods for dosing a therapeutic agent, such as in a clinical trial, e.g., Phase I clinical trial, where the dose level of the therapeutic agent is selected based on instructions that provide dose recommendations based on toxicity and efficacy outcome probabilities in a previously treated subject, or cohort of subjects. In some embodiments, the method is carried out by or with input from a non-statistician, such as a physician or clinician. In some embodiments, a statistician provides instructions containing dose recommendations based on a combination of toxicity and probability intervals. In some embodiments, one or more steps of the methods are carried out by a computer. In some embodiments, the present methods provide one or more advantages over known methods.

Generally, traditional clinical trials, e.g., Phase I dose-finding trials, e.g., oncology trials, aim to identify a maximum tolerated dose (MTD), which is the dose for which the probability of toxicity is closest to a pre-specified target toxicity rate. Generally, patients are usually enrolled and treated in a cohort size of 3, and the escalation or de-escalation decision for the next cohort of patients is based on the observed DLTs from the current cohort of patients. In some cases, such designs provide rules for escalation and de-escalation in order to identify the MTD quickly without exposing patients to excessive toxicity.

In some embodiments, some commonly used methods for performing clinical trials, e.g., dose-finding trials, include rule-based designs (e.g., 3+3 design; Storer et al. (1989) Biometrics, 45:925-937), and model-based designs (e.g., continual reassessment method; O'Quigley et al. (1990) Biometrics, 46:3348). Under the conventional 3+3 design, patients are treated in a cohort size of 3. If there is no DLT in the initial group of 3 patients, the dose is escalated to the next higher dose. If ≥2 out of the 3 patients treated at a given dose experience a DLT, the trial should stop. If one patient of the 3 treated at a given dose level experiences a DLT, an additional 3 patients are treated at the same dose level. If a DLT has occurred in exactly one of these 6 patients, escalation stops and the current dose is declared to be the MTD. In some cases, this algorithm is simple, transparent and costless to implement in practice; and clinicians can design such a study without consulting a statistician. However, the design ignores uncertainty, has no formal statistical justification, is unreliable for identifying the MTD (Ji et al. (2013) Journal of Clinical Oncology, 31:1785-1791), and increases the risk of choosing an ineffective dose. Despite this, the 3+3 design remains a dominant method for designing Phase I clinical trials.

In some embodiments, model-based design methods have provided an alternative design. In some aspects, model-based design uses Bayesian statistical methods to adaptively assign the next cohort of patients based on a dose-toxicity model that utilizes toxicity data from all treated patients across dose levels. The model-based design usually requires extensive pre-planning to fine tune priors and conduct comprehensive simulation studies to verify design operating characteristics. The model-based design can be difficult to understand for clinicians and lacks standard software for implementation, thus hindering its widespread use in practice.

In another approach, a calibration-free modified toxicity probability interval (mTPI) design) has been used (See Ji et al. Clin Trials. 2010 Dec. 7(6):653-63; Ji et al. Journal of Clinical Oncology 31.14 (2013): 1785-1791). In some aspects, in this approach the idea is that the model does not involve knowledge of a single target toxicity, which often cannot be readily identified; rather, the clinicians may have a range of acceptable toxicity intervals in mind that are used as toxicity probability intervals for statistical inference. In some embodiments, mTPI is attractive because all possible dose-assignment actions can be tabulated upfront, thus allowing clinicians to examine, calibrate and/or “buy-in” these decisions and to monitor their trials without any subsequent statistical input. In some aspects, the mTPI is a Bayesian approach, and can be viewed as a middle ground between the rule- and model-based designs. Generally, a web tool, NextGen-DF (Next-Generation Dose Finding), implements mTPI, CRM, and 3+3 for Phase I dose escalation studies.

Typically, the above methods consider the dose-limiting toxicity (DLT) data only and implicitly assume a monotone relationship between dose and efficacy response. In general, this may be a reasonable assumption for cytotoxic agents; however, this may not be true for adoptive cell therapy or other immunotherapies, such as chimeric antigen receptor (CAR) T cells or gene therapy. In some cases, adoptive cell therapy induced rapid responses, such that the monotonic dose-response assumption is unlikely to hold true or apply to such a therapy. For example, in adoptive cell therapy, the MTD is not always optimal and clinical response correlates less with dose level. Phase I trials of CAR T cells indicate that a range of dose levels is generally safe and effective, and there may be no correlation between T-cell dose and clinical response (See Davila et al. Oncoimmunology 1.9 (2012): 1577-1583). For example, in one study, two patients received a 10-fold higher T cell dose than a patient who achieved a complete response (CR) yet exhibited inferior outcomes (Kochenderfer et al. (2010) Blood, 116:3875-86; Porter et al. (2011) N Engl J. Med., 365:725-33). In another study, a TIL therapy for metastatic cancer found no correlation between the number of cells administered and the likelihood of a clinical response, with some responding patients receiving one log fewer cells than others (Johnson et al. (2009) Blood, 114:53546). Further, in some cases, unlike cytotoxic agents, substantial toxicities that will permit MTD identification may not occur in the expected or feasible therapeutic range of the gene therapies. Therefore, in some instances, the intent of dose/schedule exploration may be to determine the optimal biologically active dose and regimen (See Hussain et al. Cancer Gene Therapy (2015) 22, 554-563).

Therefore, traditional algorithmic and model-based designs for ACT dose finding based on binary measures of toxicity may be inappropriate for identifying the optimal dose. In some cases, it is sensible to incorporate efficacy or biomarker data for dose escalation or de-escalation decisions to simultaneously optimize efficacy, e.g., antitumor activity, and safety, e.g., DLT. In some aspects, this approach is useful where efficacy outcomes and toxicity outcomes can be assessed within the same timeframe.

In some cases, adoptive cell therapies, including cell therapies with chimeric antigen receptors (CARs), T cell receptors (TCR) and tumor infiltrating lymphocytes, can induce rapid responses such that toxicity and efficacy can be measured in the same timeframe. For example, based on emerging data, CAR T cell therapy can induce rapid responses so that toxicity and efficacy or one or more biomarker thereof can be measured in the same timeframe (See Davila et al. Oncoimmunology 1.9 (2012): 1577-1583; Park et al. Methods 2015 August; 84:3-16; Rebecca 2015). In some cases, in TIL therapy, the binary efficacy endpoint of persistence within 30 days post-infusion can be used as a surrogate for efficacy measure. Thus, this information allows the investigator to learn not only about the toxicity profile but also the therapeutic efficacy potential of a dose. Further, since adoptive cell therapies can be complex and expensive, it is efficient to consider capture effective biological activity rather than dose-limiting toxicity during preliminary dose exploration.

Although Phase I trials typically focus on safety, it may be important to explore the early efficacy or biomarker data before selecting a Phase II dose. Generally, due to the limitation of small sample size, dose expansion cohorts (DEC) in Phase I trials are increasingly common with the objectives to strengthen the safety evaluation, and evaluate efficacy information (See Manji et al. J Clin Oncol. 2013 Nov. 31(33):4260-7; See Dahlberg et al. J Natl Cancer Inst. 2014 Jun. 24; 106(7). pii: dju163). However, in general, little work exists in the literature to statistically justify the timing and size of the DEC. Typically, monitoring and use of the toxicity data for MTD estimation is rarely planned, and in some cases no interim analysis for futility is planned. In some cases, moving forward with a safe but futile dose into the DEC may delay the drug development process, and may also be unethical for cancer patients who are in great need of promising treatment.

In some respects, model-based methods have been developed to model toxicity and efficacy data jointly in order to determine the acceptable dose level (Thall and Cook, 2004). In some aspects, these models are complicated and difficult to understand by non-statisticians and cannot be easy to implement in practice. Furthermore, these model-based methods require large sample size and determination prior values of the toxicity and efficacy probabilities for the candidate doses, and the performance of these methods will heavily depend on the agreement of these prior estimates and the true rates. A poorly elicited set of prior estimates can lead to poor operating characteristics. In some cases, the elicitation of priors for adoptive cell therapy can be more challenging due to the highly personalized nature, including in vivo differences due to difference in tumor antigen burden or product attributes.

Provided herein are methods that consider both toxicity and efficacy in the dose recommendation decision. In some aspects of the provided methods, an optimal dose level can be selected by jointly considering safety, e.g. dose-limiting toxicity (DLT), and efficacy, e.g., response rate. In some embodiments, the provided design is not to find the maximum tolerated dose but rather the dose with the most desirable outcome for safety and efficacy. In some embodiments, the design includes one or more of the following features: 1) incorporates both toxicity and efficacy data; 2) provides the same adaptive feature as model-based design and mTPI (i.e., Bayesian); 3) is as simple to implement as 3+3 and mTPI design, while being transparent and easy for non-statisticians to understand.

In aspects of the provided method, after each cohort of patients complete the DLT evaluation period, rather than taking one of three possible actions (escalate, de-escalate or stay at the current dose level) based on toxicity data, the trade-off between toxicity and efficacy is considered to make the dosing design. In some embodiments, this assumes the monotonic relationship between efficacy and toxicity may not be held. In some embodiments, decision rules can be pre-specified prior to the start of the trial, allowing for transparency to clinicians and non-statisticians. In some cases, the approach provides adaptive features and is easy and transparent to implement. In some embodiments, the method can be adapted to design clinical trials, e.g. Phase I dose-finding trials, for testing any therapy for which toxicity and efficacy response can be assessed in the same timeframe. In some instances, once the trial is complete, the dose level with the largest combined safety and efficacy utility is chosen as the optimal dose level.

### II. Design of Toxicity and Efficacy Probability Interval (TEPI) Model

Provided herein in some embodiments is a toxicity and efficacy probability interval (TEPI) design for determining the dose of a therapeutic agent (e.g. cell therapy, such as CAR T cell therapy), where safety and efficacy data can be observed in approximately the same timeframe or period. In some embodiments, the provided methods can be used in connection with performing a clinical trial, such as a Phase I dose-finding trial (such as for cancer immunotherapies). In some embodiments, the TEPI design is an extension over the mTPI, and utilizes both safety and efficacy data to inform dose escalation decisions. In some aspects, TEPI may allow the dose-expansion cohort to be built in as part of the overall study design so that the overall study operating characteristics can be evaluated, and safety and efficacy data can be borrowed across all tested dose levels for the final toxicity and efficacy estimation. In some aspects, decision rules (dose-finding protocol) can be pre-specified prior to the start of the trial, allowing for transparency to clinicians and non-statisticians.

In some embodiments, the method involves obtaining a matrix, such as generating a matrix or providing a generated matrix, that comprises one or more dosing actions associated with a combination of toxicity and efficacy probability intervals, for example, associated with or that may be associated with a therapeutic agent being used in a clinical trial. In some aspects, the method includes determining a joint unit probability mass (UPM) for each combination of toxicity and efficacy probability intervals for one or more true or possible toxicity probabilities at a current dose level i (pᵢ) and one or more true or possible efficacy probabilities at current dose level i (qᵢ). In some embodiments, pᵢ is a ratio of a number of subjects experiencing a toxic outcome (xᵢ) to a total number of subjects (nᵢ), and qᵢ is a ratio of the number of subjects experiencing a response outcome (yᵢ) to the total number of subjects (nᵢ).

In some embodiments, the toxicity outcome is a dose-limiting toxicity (DLT). In some cases, the toxicity outcome is the presence or absence of one or more biomarkers, a value or level of one or more biomarkers, persistence of cells within 30 to 60 days or more post-infusion or other parameter of activity of the therapy.

In some embodiments, the response outcome is a complete response (CR). In some cases, the response outcome is the presence or absence of one or more biomarkers or is a value or level of one or more biomarkers.

In some cases, the method involves identifying the combination of toxicity and efficacy intervals that has the highest joint UPM. In some embodiments, the method includes assigning the dosing action associated with the identified combination as a dose recommendation for each of the one or more true or possible toxicity and efficacy probabilities.

In some cases, the method involves producing instructions that specify the dose recommendations. In some embodiments, the instructions include or are contained in one or more tables that specify the dose recommendations, such as a dose recommendation decision table. In some embodiments, dose recommendations are provided for one or more possible combinations of xᵢ and yᵢ among m subjects. In some aspects, dose recommendations are provided for all possible combinations of and yᵢ among m subjects. In some embodiments, m is within a range from about 1 to about 100, about 3 to about 60, or about 6 to about 30. In some cases, m is or is about 1, 3, 6, 9, 12, 15, 18, 21, 24, 27, 30, 45, 75, 99, or more.

In some embodiments, the clinical trial is a Phase I clinical trial. In some instances, the clinical trial is a Phase I/II clinical trial.

In some embodiments, the method is a computer implemented method, and one or more steps occur at an electronic device comprising one or more processors and memory. In some aspects, the electronic device, e.g., computer system comprising a processor and memory, the memory contains instructions operable to cause the processor to carry out one or more of steps of the method.

In certain embodiments, methods provided herein are computer implemented methods and/or are performed with the aid of a computer. In some embodiments, provided herein are methods for providing a dose recommendation for a therapeutic agent, for example in a clinical trial, by computer implemented methods and/or by methods which include steps that are computer implemented steps. In some embodiments, obtaining a matrix comprising one or more dosing actions associated with a combination of toxicity and efficacy probability intervals is implemented by a computer. In particular embodiments, determining a joint unit probability mass (UPM) for each combination of toxicity and efficacy probability intervals for one or more possible toxicity probabilities at current dose level i (pᵢ) and one or more possible efficacy probabilities at current dose level i (qᵢ) is implemented on a computer. In certain embodiments, identifying the combination of toxicity and efficacy intervals that has the highest joint UPM is implemented by computer. In some embodiments, assigning the dosing action associated with the identified combination as a dose recommendation for each of the one or more possible toxicity and efficacy probabilities is implemented by a computer. In certain embodiments, producing or outputting instructions that specify the dose recommendations is implemented by a computer. In particular embodiments, a computer system comprising a processor and memory is provided, wherein the memory contains instructions operable to cause the processor to carry out any one or more of steps of the methods provided herein.

In certain embodiments, methods provided herein may be practiced, at least in part, with computer system configurations, including single-processor or multi-processor computer systems, minicomputers, mainframe computers, as well as personal computers, hand-held computing devices, microprocessor-based and/or programmable consumer electronics and the like, each of which may operatively communicate with one or more associated devices. In particular embodiments, the methods provided herein may be practiced, at least in part, in distributed computing environments such that certain tasks are performed by remote processing devices that are linked through a communications network. In a distributed computing environment, program modules may be located in local and/or remote memory storage devices. In particular embodiments, some or all steps of the methods provided herein may be practiced on stand-alone computers.

In particular embodiments, some or all of the steps of the methods provided herein can operate in the general context of computer-executable instructions, such as program modules, executed by one or more components. Generally, program modules include routines, programs, objects, data structures, etc., that perform particular tasks or implement particular abstract data types. Typically, the functionality of the program modules may be combined or distributed as desired. In certain embodiments, instructions operable to cause the processor to carry out any one or more steps of the methods provided herein can be embodied on a computer-readable medium having computer-executable instructions and transmitted as signals manufactured to transmit such instructions as well as the results of performing the instructions, for instance, on a network.

A. Matrix (e.g., Preset Table)

In some embodiments, the TEPI model includes creating, obtaining, or receiving a matrix, e.g. preset table, that contains one or more dosing actions associated with a combination of toxicity and efficacy probability intervals. In some embodiments, the matrix is displayed in a two-way grid or table, such as a preset table, constructed by combining probability intervals of toxicity and efficacy. In some cases, the toxicity and/or efficacy probability intervals are obtained from or designated by a clinician or physician or with input from a clinician or physician.

In some embodiments, in order to create a matrix, e.g. preset table, it is necessary to have information about (1) the target toxicity rate, p_{T}, such as the toxicity rate at which a clinician is comfortable to treat future patients at the current dose if there is an acceptable efficacy outcome to justify the risk-benefit trade-off; and (2) the minimum acceptable efficacy (e.g. antitumor activity) rate, q_{E}, such as the minimum acceptable efficacy rate at which the clinician is willing to treat future patients at the current dose level. In some embodiments, the matrix is created by designating two or more toxicity probability intervals of the therapeutic agent and two or more efficacy probability intervals of the therapeutic agent, which are based on the information about the p_{T} and q_{E}, respectively. In some aspects, a dosing action is assigned to each combination of toxicity and efficacy probability intervals contained within the matrix (e.g., preset table).

In some embodiments, a maximum acceptable toxicity probability or rate (p_{T}) is obtained or specified. In some cases, p_{T} is considered in the design of the toxicity probability intervals (e.g., the range of values contained within one or more of the intervals). For example, one of the toxicity probability intervals may specify a range of values containing p_{T} and other toxicity probability intervals may have ranges above or below that in which p_{T} is contained.

In some embodiments, a minimum acceptable efficacy probability or rate (q_{E}) is obtained or specified. In some cases, q_{E} is considered in the design of the efficacy probability intervals (e.g., the range of values contained within one or more of the intervals). For example, one of the efficacy probability intervals may specify a range of values containing q_{E} and other efficacy probability intervals may have ranges above or below that in which q_{E} is contained.

In some cases, each toxicity or probability interval contains a range of values. In some embodiments, the range of values contained within all of the toxicity probability intervals and/or all of the efficacy probability intervals is from 0 to 1. Thus, in some cases, the range of values contained within each toxicity or efficacy probability interval is a subset within the range of 0 to 1.

In some embodiments, the matrix includes two or more toxicity probability intervals, such as 2, 3, 4, 5, 6, 7, 8, 9, 10, or more toxicity probability intervals. In some aspects, the matrix comprises two toxicity probability intervals. In some aspects, the matrix comprises three toxicity probability intervals. In some cases, the matrix comprises four toxicity probability intervals.

In some embodiments, the matrix contains two or more efficacy probability intervals, such as 2, 3, 4, 5, 6, 7, 8, 9, 10, or more efficacy probability intervals. In some aspects, the matrix comprises two efficacy probability intervals. In some aspects, the matrix comprises three efficacy probability intervals. In some cases, the matrix comprises four efficacy probability intervals.

In some embodiments, the two unit intervals of (0, 1) for pᵢ and qᵢ are partitioned into subintervals. In some aspects, (a, b) and (c, d) are denoted as subintervals for the partition of pᵢ and qᵢ, respectively. Thus, in some embodiments, each toxicity probability interval is defined by a start value a and an end value b. In some aspects, each efficacy probability interval is defined by a start value c and an end value d. This, in some aspects, the intersection of toxicity probability interval and efficacy probability interval is defined as (a, b)×(c, d). In some embodiments, the methods include a dose-finding protocol that involves input of physician-specified decisions for a range of {(a; b); (c; d)} values to anchor the statistical inference.

In some embodiments, the interval (0, 1) is partitioned into at least or about at least or three parts, such as depending on the equivalence interval (EI): (1) (0, p_{T}−ε₁), which in some cases can denote dosing action “E”; (2) (p_{T}−ε₁, p_{T}+ε₂), which in some cases can denote dosing action “S”; or (3) (p_{T}+ε₂, 1), which in some cases can denote dosing action “D.” In some embodiments, p_{T}−ε₁ is the lowest toxicity probability that the physician would be comfortable using to treat future patients without dose escalation. In some embodiments, p_{T}+ε₂ is the highest toxicity probability that the physician would be comfortable using to treat future patients without dose de-escalation. As an example, if p_{T}=0.3, ε₁=0.05, then the equivalence interval (EI) is 25-35%. In some embodiments, as described below, the dosing action “E”, “D” or “S” is decided based on which interval has the highest unit probability posterior mass (UPM).

In some embodiments, a two-way grid or table, e.g., preset table,) is created, e.g., as shown in Table 1, in which the interval combination (a, b)×(c, d) forms the basis for dose finding decisions, with each combination corresponding to a specific decision, e.g. dose escalation. In some cases, the two-way grid or table, e.g., preset table, is designed by or with input from physicians or clinicians. In some cases, each toxicity probability interval is assigned a toxicity grade, such as “Low”, “Moderate”, “High”, or “Unacceptable”, e.g., as shown in Table 1. In some cases, each efficacy probability interval is assigned an efficacy grade, such as “Low”, “Moderate”, “High”, or “Superb”, e.g., as shown in Table 1. In some embodiments, the preset table can be depicted by two-dimensional rectangles that result from the combinations or intersections of toxicity probability interval and efficacy probability interval.

TABLE 1

| | | | Efficacy Rate (qE) | | | |
|---|---|---|---|---|---|---|
| | | | Low | Moderate | High | Superb |
| | | | (c_{Low}, d_{Low}) | (c_{Mod}, d_{Mod}) | (c_{High}, d_{High}) | (c_{Sup}, d_{Sup}) |
| Toxicity | Low | (a_{Low}, b_{Low}) | EU/E/S/D/DU | EU/E/S/D/DU | EU/E/S/D/DU | EU/E/S/D/DU |
| Rate | Moderate | (a_{Mod}, b_{Mod}) | EU/E/S/D/DU | EU/E/S/D/DU | EU/E/S/D/DU | EU/E/S/D/DU |
| (pᵢ) | High | (a_{High}, b_{High}) | EU/E/S/D/DU | EU/E/S/D/DU | EU/E/S/D/DU | EU/E/S/D/DU |
| | Unacceptable | (a_{Un}, b_{Un}) | EU/E/S/D/DU | EU/E/S/D/DU | EU/E/S/D/DU | EU/E/S/D/DU |

In some instances, in the preset table, a combination of toxicity and efficacy intervals (and thus toxicity and efficacy grades) is associated with or assigned a dosing action. For example, in Table 1, there are 16 combinations (e.g. two-dimensional rectangles), and each rectangle corresponds to one or more specific dosing actions. In some embodiments, dosing action “E” denotes escalation, i.e., treating subjects at the next higher dose level (i+1) as compared with the current dose level i. In some aspects, dosing action “S” denotes staying at the current dose level i for future subjects. In some cases, dosing action “D” denotes de-escalation, i.e., treating subjects at the next lower dose level (i−1) as compared with the current dose level i. In some instances, dosing action “DU” encompasses “D” and “U”, which indicates that the current dose level is unacceptable due to high toxicity and will be excluded in the trial for the following cohorts. In some embodiments, these dosing actions reflect practical clinical actions when the safety and efficacy data are observed at a certain dose level.

For instance, as shown in Table 1, in some cases, a Low-Toxicity and Low-Efficacy corresponds to a two-dimensional rectangle, (a_{Low}, b_{Low})×(c_{Low}, d_{Low}) (e.g. (0. 0.15)×(0, 0.2)) for pᵢ and qᵢ respectively, in which the dosing action is “E” or escalation. In some aspects, this indicates that if the observed toxicity rate falls within (a_{Low}, b_{Low}) (e.g. 0, 0.15) and the observed efficacy rate falls within (c_{Low}, d_{Low}) (e.g. 0, 0.2), the next cohort would be treated at a higher dose level. Thus, in some cases, the dosing action is relative to the current dose level i and specifies a dosing action to be taken in the next subject or cohort of subjects.

In some embodiments, the preset table can be based on one or more rationales associated with one or more expected outcomes of a therapeutic agent. In some embodiments, if the current dose level is associated with low toxicity, the dosing action decision for the next dose for the next cohort would be escalated regardless of the efficacy. In some aspects, if the current dose level is associated with moderate toxicity, the dosing action may be “E” or “S” depending on the efficacy outcome. In some cases, such as where the desired efficacy has been achieved at the current dose level, clinicians may choose “S” instead of “E” to avoid unnecessarily exposing additional subjects to higher toxicity. In some instances, this may allow for rapid accrual and allocation of patients at this promising dose level.

In some embodiments, if the current dose level is associated with high (but acceptable) toxicity, the dosing action may be “D” or “S” depending on the efficacy outcome. In some cases, the upper bound of this toxicity interval represents the maximum acceptable toxicity rate, p_{T}. In some embodiments, if the risk-benefit can be justified, the dosing action may be “S” at the current dose level. In some instances, this is a logical action in that if the therapy at the MTD is ineffective, but a higher dose has greater efficacy and is also acceptably safe, then the higher dose should be explored. For example, in some cases, such as where subjects have failed standard therapies, it may be worthwhile to further investigate a dose with moderate or high toxicity by assigning more subjects to it. In some aspects, additional safety rules can be introduced to exclude the dose if toxicity becomes unacceptable. In some aspects, if the current dose level exceeds the maximum tolerable toxicity, the dosing action in “D” because the safety risk is unacceptably high.

B. Dose Recommendation Instructions

In some embodiments, provided are methods for producing, preparing, and/or providing dose recommendation instructions. In some embodiments, the instructions are displayed in a table, such as a dose recommendation decision table. In some embodiments, the dose recommendations displayed in the table are determined by a dose-finding protocol, such as those described herein and/or other protocol based on the TEPI model described herein.

1. Joint Unit Probability Mass

In some embodiments, based upon the preset Table described above, a dose-finding protocol (e.g. Table) is derived that provides dose recommendation instructions. In some embodiments, building upon the preset table, a local decision-theoretic framework is set up. In some instances, local means that the framework focuses on the optimal decision to be made for the current dose, instead of the trial. In some aspects, a Bayes rule is derived. In some aspects, the design depends on the joint UPM (JUPM) of toxicity and efficacy data, which follows the Bayes rule under independent beta prior distributions. Thus, in some cases, the Bayes rule is equivalent to computing the JUPM for the toxicity and efficacy probability intervals.

In some embodiments, the dose recommendations can be pre-specified based on true (e.g., observed) toxicity and efficacy probabilities. In some embodiments, the dose recommendations can be pre-specified based on unknown toxicity and efficacy probabilities. Thus, in some embodiments, the methods include determining a joint unit probability mass (UPM) for each combination of toxicity and efficacy probability intervals for one or more true or possible toxicity probabilities at current dose level i (pᵢ) and one or more true or possible efficacy probabilities at current dose level i (qᵢ).

In some embodiments, for a given region A, the joint UPM can be defined as the ratio between the probability of the region (i.e., interval, e.g. E, S or D) and the size of the region (i.e., interval, e.g. E, S or D).

In some embodiments, the clinical trial includes d ascending doses and pᵢ and qᵢ denote the true or unknown probability of toxicity and efficacy for the i^{th} dose, respectively. In some embodiments, it is assumed that the toxicity probability pᵢ increases with dose level i. In some aspects, the efficacy probability qᵢ may increase with dose level i or may increase initially and then reach a plateau or show minimum improvement at higher dose levels (See Davila et al. Oncoimmunology 1.9 (2012): 1577-1583; Park et al. Methods 2015 August; 84:3-16). In some aspects, the efficacy probability qᵢ may decrease with dose level i. Thus, in some cases, it is assumed that pᵢ and qᵢ are independent. In some aspects, dose i is currently used in the trial and nᵢ subjects have already been allocated to dose i, with xᵢ and yᵢ subjects experiencing a toxicity or efficacy outcome, respectively. In some embodiments, it is assumed that xᵢ and yᵢ are independently distributed. In some embodiments, the trial data is denoted as D={(nᵢ, xᵢ, yᵢ), i=1, . . . , d}.

In some embodiments, the joint UPM is determined for a given combination of toxicity and efficacy probability intervals by determining the probability that pᵢ and qᵢ are contained within the combination of toxicity and probability intervals and dividing by the product of the toxicity probability interval length and the efficacy probability interval length.

In some embodiments, considering the two-dimensional unit square (0, 1)×(0, 1) in the real space, the joint UPM (JUPM) for the rectangular region of (a, b)×(c, d) is:

$$JUPM \begin{matrix}( c , d ) \\ ( a , b )\end{matrix} ≡ \frac{Pr ( {p}_{i} ∈ ( a , b ) , {q}_{i} ∈ ( c , d ) \vert 𝒟 )}{( b - a ) \times ( d - c )} , \\ 0 < a < b < 1 , 0 < c < d < 1. \qquad ( 1 )$$

In some aspects, the numerator of formula (1) is the posterior probability of pᵢ and qᵢ in the interval (a, b) and (c, d), respectively. Thus, in some aspects, determining the joint UPM is based on the posterior distributions of pᵢ and qᵢ, according to Bayes' rule, as described above.

In some embodiments, it is assumed that pᵢ and qᵢ have a beta prior distribution Beta(x; α, β), and that the posterior distributions for pᵢ and qᵢ are independent and follow Beta(x; α+xᵢ, β+nᵢ−xᵢ) and Beta(x; α+yᵢ, β+nᵢ−yᵢ), respectively.

Thus, in some embodiments, the priors for each pᵢ follow the independent beta(αₚ, βₚ), and the priors for each qᵢ follow independent beta(α_{q}, β_{q}). In some embodiments, since xᵢ˜Bin(nᵢ, pᵢ) and yᵢ˜Bin(nᵢ, qᵢ), the likelihood function for toxicity data (xᵢ, nᵢ), i=1, . . . , d is a product of binomial densities:

l(p)=Πᵢ₌₁^{d}pᵢ^{x}^{ᵢ}(1−pᵢ)ⁿ^{ᵢ}^{-x}^{ᵢ}.

In some cases, the likelihood function for efficacy data (y₁, nᵢ), i=1, . . . , d is a product of binomial densities:

l(q)=Πᵢ₌₁^{d}pᵢ^{x}^{ᵢ}(1−qᵢ)ⁿ^{ᵢ}^{-x}^{ᵢ}.

In some embodiments, the toxicity-efficacy two-dimensional space (0, 1)×(0, 1) could be split into sixteen regions:

Rec_{g,h}=I_{1,g}×I_{2,h},g=1, 2, 3, 4,h=1, 2, 3, 4, where I_{1,g}=(a_{g-1},a_{g})

and

I_{2,h}=(bₕ₋₁,bₕ).

In some cases, a₀=b₀=0, a₁=0.15, a₂=0.33, a₄=b₄=1, b₁=0.2, b₂=0.4, and/or b₃=0.6. In some aspects, each region corresponds to a deterministic best decision. For example, in some instances, a* is the interval estimation of the variable pair (pᵢ, qᵢ) among a fixed action set A={Rec_{g,h}, g=1, 2, 3, 4; h=1, 2, 3, 4}.

In some aspects, under the loss function (2)

$$l ( {a}^{*} = {Rec}_{g , h} , {p}_{i} , {q}_{i} ) = \{ \begin{matrix}0 , & {p}_{i} ∈ {I}_{1 , g} and {q}_{i} ∈ {I}_{2 , h} , \\ \frac{1}{( {a}_{{g}_{o}} - {a}_{{g}_{o} - 1} ) \times ( {b}_{{h}_{o}} - {b}_{{h}_{o} - 1} )} , & {p}_{i} ∈ {I}_{1 , {g}_{o}} and {q}_{i} ∈ {I}_{2 , {h}_{o}} , \\ & where {g}_{o} \neq g , {h}_{o} \neq h .\end{matrix} \qquad ( 2 )$$

and uniform prior for both pᵢ and qᵢ, if (g*, h*) is the index pair of the region among A that has the minimum joint UPM (JUPM), i.e.:

$$( {g}^{*} , {h}^{*} ) = arg {max}_{g ∈ \{ 1 , 2 , 3 , 4 \} , h ∈ \{ 1 , 2 , 3 , 4 \}} JUPM \begin{matrix}{b}_{h - 1} , {b}_{h} \\ {a}_{g - 1} , {a}_{g}\end{matrix} ,$$

the Bayes rule is given by:

B=Rec_{g*,h*}. (3)

In some embodiments, the proof for the above theorem is as follows:
Proof: Define the posterior expected loss for any

$${a}^{*} = {Rec}_{\overline{g} , \overline{h}} ∈ A$$

by

$$\begin{matrix}R ( {a}^{*} , 𝒟 ) = {E}_{pi , qi} [ l ( {a}^{*} , {p}_{i} , {q}_{i} ) \vert 𝒟 ] \\ = {\int }_{0}^{1} {\int }_{0}^{1} l ( {a}^{*} , {p}_{i} , {q}_{i} ) π ( {p}_{i} , {q}_{i} ) \vert 𝒟 ) {dp}_{i} {dq}_{i} \\ = {\sum }_{g = 1}^{4} {\sum }_{h = 1}^{4} {\int }_{{a}_{g - 1}}^{{a}_{g}} {\int }_{{b}_{h - 1}}^{{b}_{h}} l ( {a}^{*} , {pi}_{i} , {q}_{i} ) π ( {p}_{i} , {q}_{i} \vert 𝒟 ) {dp}_{i} {dq}_{i} \\ = {\sum }_{( g , h ) \neq ( \overline{g} , \overline{h} )} \frac{Pr ( {p}_{i} ∈ ( {a}_{g - 1} , {a}_{g} ) , {q}_{i} ∈ ( {b}_{h - 1} , {b}_{h} ) \vert 𝒟 )}{( {a}_{g} - {a}_{g - 1} ) \times ( {b}_{h} - {b}_{h - 1} )} \\ = {\sum }_{( g , h ) \neq ( \overline{g} , \overline{h} )} JUPM \begin{matrix}{b}_{h - 1} , {b}_{h} \\ {a}_{g - 1} , {a}_{g}\end{matrix} .\end{matrix}$$

In some aspects, the Bayes rule that achieves the minimum posterior expected loss is given by:

$$\vert = arg {min}_{{a}^{*} ∈ A} R ( {a}^{*} , 𝒟 )$$

which in some cases is equivalent to (3),

$$since above = constant - JUPM \begin{matrix}{b}_{\overline{h} - 1} , {b}_{\overline{h}} \\ {a}_{\overline{g} - 1} , {a}_{\overline{g}}\end{matrix} , \\ where constant = {\sum }_{g ∈ \{ 1 , 2 , 3 , 4 \} , h ∈ \{ 1 , 2 , 3 , 4 \}} JUPM \begin{matrix}{b}_{h - 1} , {b}_{h} \\ {a}_{g - 1} , {a}_{g}\end{matrix} .$$

Thus, in some embodiments:

$$B = {t}_{{g}^{*} , {h}^{*}} , \\ where ( {g}^{*} , {h}^{*} ) = arg {max}_{g ∈ \{ 1 , 2 , 3 , 4 \} , h ∈ \{ 1 , 2 , 3 , 4 \}} JUPM \begin{matrix}{b}_{h - 1} , {b}_{h} \\ {a}_{g - 1} , {a}_{g}\end{matrix} .$$

In some embodiments, as each action:

a*ϵA

corresponds to one deterministic decision of E, S, or D, as shown in the preset table, the trial proceeds with the corresponding decision of B, which is the Bayes rule.

In some embodiments, some of the variables in the formulas shown herein may be represented by alternative terms. For example, in some aspects, the term Rec can be represented by the term t. In some examples, the term a* can be represented as the term x. Thus, the terms Rec and t can be used interchangeably and the terms a* and x can be used interchangeably.

Based on the posterior distributions, there is a winning rectangle, e.g., combination of toxicity and probability intervals, (a*, b*)×(c*, d*) that achieves the maximum joint UPM among all the rectangles in a preset table, and corresponding dosing actions can be provided as dose recommendations for treating the next cohort of subjects. Thus, in some aspects, the interval with the highest joint UPM results in the escalation (E), stay (S) or de-escalation (D) decision. Such a decision is the Bayes' Rule under 0/1 loss function.

Thus, in some aspects, the TEPI model assumes that a current patient cohort is treated at dose i and after the current cohort of patients completes DLT and response evaluation, the JUPMs for all the interval combinations in the preset table are calculated. In some such cases, the TEPI design recommends “E”, “S”, or “D” corresponding to the combination with the largest JUPM value. In some aspects, based on the preset table, all of the decisions can be precalculated and presented in the dose recommendation decision table. Such a table may allow clinicians to conduct the trial with transparency.

In some embodiments, the dose recommendations can be pre-determined for all the trial data considering any of a number of possible combinations of toxicity and efficacy outcomes. In some cases, the decisions are compiled into a dose recommendation decision table to pre-specify the dosing decisions for clinicians and non-statisticians.

2. Safety and Futility Rules

In some embodiments, the dose-finding protocol includes two additional rules. In some aspects, a safety rule is included to exclude dose levels with excessive toxicity. In some cases, a futility rule is included to exclude dose levels with very low efficacy.

An exemplary safety rule is: if Pr(pᵢ>p_{T} (data)>η, exclude dose i, i+1, . . . , d for further use (i.e. dose will never be tested again the trial, e.g. corresponding to action “DU” or do not return to the current dose level or any higher dose level due, in some cases due to unacceptable toxicity). In some embodiments, η is close to 1, such as is at least or at least about or is 0.95.

An exemplary futility rule is: if Pr(qᵢ<q_{E}|data)>ξ, exclude dose i, i−1, . . . , d for further use (e.g. corresponding to action “EU” or do not return to the current dose level or any lower dose level, in some cases due to unacceptable low efficacy). In some embodiments, ξ is small, such as is less than or less than about or about 0.3.

In the above exemplary rules, p_{T} is the highest toxicity rate that can be tolerated and q_{E} is the lowest efficacy rate that is deemed effective. In some embodiments, a dose satisfying both rules is considered an “available” dose. In some cases, only available doses can be used to treat subjects in the trial. In some aspects, the two rules and the Bayes' Rule that maximizes joint UPM are combined and a trial design dose-finding protocol is presented.

In some embodiments, the dose recommendation is escalate (E) to dose level i+1. In some embodiments, the dose recommendation is stay (S) at dose level i. In some cases, the dose recommendation is de-escalate (D) to dose level i−1. In some aspects, the dose recommendation is de-escalate and do not return to the current dose level or any higher dose level, in some cases due to unacceptable toxicity (denoted interchangeably as either DU or DU_{T}). In some embodiments, the dose recommendation is de-escalate and do not return to the current dose level or any higher dose level due, in some cases due to unacceptable low efficacy (denoted interchangeably as either DEU or DU_{E}). In some cases, the dose recommendation is escalate and do not return to the current dose level or any lower dose level, in some cases due to unacceptable low efficacy (denoted interchangeably as either EEU or EU).

In some embodiments, dose recommendation instructions are generated (e.g. a dose-finding Table). In some embodiments, all of the some embodiments, all of the dose finding decisions, e.g. during a clinical trial, simply follow the dose recommendation instructions. Exemplary dose recommendations instructions are set forth in Tables 5 and 6.

3. Trial Conduct Using TEPI

In some embodiments, a dose-finding protocol, (e.g., algorithm) that takes into account the maximum joint UPM and the safety and futility rules is used to provide dose recommendations. In some embodiments, a dose-finding protocol that takes into account the safety and futility rules is used to alter provided dose recommendations based on corresponding dosing actions specified in the preset table. Thus, in some aspects, the dose recommendations provided using a protocol that includes the safety and futility rules may differ from the dosing actions specified in the preset table for a corresponding decision based on a protocol without the inclusion of the safety and futility rules.

In some embodiments, a starting dose is chosen, which, in some cases, can take into account the maximum tolerable toxicity rate (p_{T}) and the minimum acceptable efficacy rate (q_{E}). In some embodiments, the starting dose is chosen by the clinician or physician treating the subject. In some embodiments, a matrix, e.g., preset table, as described above (e.g. Table 1) is generated, which is then used to derive the dose recommendation decision table (e.g. Tables 5 or 6).

In some embodiments, the dose recommendation decision table reflects clinical practice during the trial. In some cases, the intervals in the preset table are calibrated as needed.

In some embodiments, a dose is available if Pr(pᵢ>p_{T} data)<η and Pr(qᵢ>qₑ| data)>ζ. In some embodiments, if no dose level is available, the trial is terminated. In some cases, the current dose level i is the dose level used to treat the current cohort of subjects.

In some aspects, if the current dose level violates the safety rule (e.g., pᵢ is greater than p_{T}), the dose level is de-escalated to the maximum available dose below the current dose. In some cases, if the current dose violates the safety rule, the dose level is de-escalated and dose level i and any higher dose levels are marked as unavailable. In some aspects, if the probability that pᵢ is greater than the maximum acceptable toxicity probability (p_{T}) exceeds 0.95, the dose level is de-escalated and dose level i and any higher dose levels are marked as unavailable.

In some embodiments, if the current dose level violates the futility rule (e.g., qᵢ is less than q_{E}), the dose level is de-escalated and dose level i and any higher dose levels are marked as unavailable. In some embodiments, if the current dose level violates the futility rule, the dose level is escalated and current dose level i and all lower doses are marked as unavailable. In some aspects, if the probability that qᵢ is less than the minimum acceptable efficacy probability (q_{E}) exceeds 0.7, the dose level is de-escalated and dose level i and any higher dose levels are marked as unavailable. In some aspects, if the probability that qᵢ is less than the minimum acceptable efficacy probability (q_{E}) exceeds 0.7, the dose level is escalated and dose i and all lower doses are marked as unavailable.

In some embodiments, if the current dose violates the futility rule, the following rules (e.g., dose-finding protocol) apply:

If the decision is “E”, escalate to the closest available dose above the current dose. If no doses above the current dose are available, de-escalate the dose to the closest available dose below the current dose. In some embodiments, this rule is justified because efficacy may not always be monotone. Therefore, in some cases, when the current dose is not effective, an effective dose could be either a higher dose or a lower dose.

If the decision is “D”, de-escalate to the closest available dose below the current dose. In no doses below the current dose are available, terminate the trial.

If the decision is “DU”, mark the current dose and all the higher doses as unavailable. If no doses are available, terminate the trial. If there are still available doses below the current dose, de-escalate the dose to the closest available dose below the current dose.

If the decision is “S”, de-escalate the dose to the closest available dose below the current dose. If no dose below the current dose is available, terminate the trial.

In some embodiments, if the current dose satisfies both the safety rule and the futility rule, the following rules (e.g., dose-finding protocol) apply:

If the decision is “E”, escalate the dose to the closest available dose above the current dose. If no doses above the current dose are available, stay at the current dose.

If the decision is “D”, de-escalate to the closest available dose below the current dose. If no doses below the current dose are available, stay at the current dose.

If the decision is “DU”, mark the current dose and all higher doses as unavailable. If no doses are available, terminate the trial. If there are still available doses below the current dose, de-escalate the dose to the closest available dose below the current dose.

If the decision is “S”, stay at the current dose.

### III. Clinical Dosing According to TEPI Model

Provided in some aspects are methods for dosing a subject, such as a human subject in a clinical trial, e.g., Phase I or Phase I/II clinical trial, or to each subject belonging to a cohort of a clinical trial. In some embodiments, the clinician chooses the starting dose, the maximum acceptable toxicity, and/or the minimum acceptable efficacy, e.g., antitumor activity. In some embodiments, the preset table is derived as described above. In some cases, the clinician reviews the preset table to ensure it reflects clinical practice during a trial. In some aspects, the intervals can be calibrated as needed.

In some embodiments, there is a close collaboration between clinicians and statisticians to determine the initial design parameters such as the interval combinations and corresponding dosing actions, e.g., as shown in Table 1, the utility function, and the safety and futility stopping rules. In some embodiments, once all decision rules are chosen, trial operating characteristics may be evaluated through simulation studies. In some cases, simulation results may be analyzed to fine tune design parameters as needed. In some instances, such a process iterates until satisfactory trial operating characteristics are achieved. In some embodiments, the computation used for the simulation is fast such that it can be completed in a few minutes due to the simple modeling framework. In some aspects, this makes the calibration process less burdensome for the trial team. In some cases, due to the transparency and simplicity of the design, calibration of these parameters in intuitive and/or requires less effort than other models.

In some cases, the method includes selecting a dose recommendation for administering a therapeutic agent to a subject based on instructions produced or outputted by the any of the methods described herein. In some aspects, the dose recommendation is selected for a given combination of a number of subjects experiencing a toxic outcome (xᵢ) and a number of subjects experiencing a response outcome (yᵢ) for a total number of subjects previously treated with the therapeutic agent in the clinical trial at a current dose level i (nᵢ). In some embodiments, the method includes administering the therapeutic agent to the subject at a dose level in accord with the selected dose recommendation.

In some embodiments, the toxicity outcome is a dose-limiting toxicity (DLT). In some embodiments, the toxicity outcome is the presence or absence of one or more biomarkers or a level of one or more biomarkers.

In some embodiments, the response outcome is a complete response (CR). In some embodiments, the response outcome is the presence or absence of one or more biomarkers or a level of one or more biomarkers.

In some embodiments, the method involves obtaining instructions that specify dose recommendations. In some cases, the instructions are prepared by designating two or more toxicity probability intervals of a therapeutic agent and two or more efficacy probability intervals of the therapeutic agent. In some instances, the instructions are prepared by assigning a dosing action to each combination of toxicity and efficacy probability intervals. In some cases, the instructions are prepared by determining, e.g., calculating, the joint unit probability mass (UPM) for each combination of toxicity and efficacy probability intervals for one or more possible toxicity probabilities at current dose level i (pᵢ) and one or more possible efficacy probabilities at current dose level i (qᵢ). In some embodiments, the instructions are prepared by identifying the combination of toxicity and efficacy intervals that has the highest joint UPM. In some cases, the instructions are prepared by assigning the dosing action associated with the identified combination as a dose recommendation for each of the one or more possible toxicity and efficacy probabilities.

In some embodiments, the methods include selecting a dose recommendation for administering a therapeutic agent to a subject based on the instructions for a given combination of a number of subjects experiencing a toxic outcome (xᵢ) and a number of subjects experiencing a response outcome (yᵢ) for a total number of subjects previously treated with the therapeutic agent in the clinical trial at a current dose level i (nᵢ). In some aspects, the method involves administering the therapeutic agent to the subject at a dose level according to the selected dose recommendation.

In some embodiments, prior to producing the instructions, the dose recommendation is altered to de-escalate and not return to current dose if the probability that pᵢ is greater than the maximum acceptable toxicity probability (p_{T}) exceeds 0.95. In some embodiments, prior to producing the instructions, the dose recommendation is altered to de-escalate and not return to current dose if the probability that qᵢ is less than the minimum acceptable efficacy probability (q_{E}) exceeds 0.7. In some embodiments, prior to producing the instructions, the dose recommendation is altered to escalate and not return to current dose if the probability that qᵢ is less than q_{E} exceeds 0.7.

In some embodiments, prior to producing the instructions, the dose recommendation is altered to de-escalate and not return to current dose if pᵢ is greater than p_{T}. In some embodiments, prior to producing the instructions, the dose recommendation is altered to de-escalate and not return to current dose if qᵢ is less than q_{E}. In some embodiments, prior to producing the instructions, the dose recommendation is altered to escalate and not return to current dose if qᵢ is less than q_{E}.

In some embodiments, the dose recommendation is de-escalate and do not return to the current dose level or any higher dose level, for example in some cases due to toxicity (denoted interchangeably as DU or DU_{T}). In some embodiments, the dose recommendation is de-escalate and do not return to the current dose level or any higher dose level, for example in some cases due to low efficacy (denoted interchangeably as DEU or DU_{E}). In some embodiments, the dose recommendation is escalate and do not return to the current dose level or any lower dose level, for example in some cases due to low efficacy (denoted interchangeably as EEU or EU).

In some embodiments, prior to obtaining instructions, the maximum acceptable toxicity probability (p_{T}) and minimum acceptable efficacy probability (q_{E}) of the therapeutic agent are designated, provided, or obtained.

In some embodiments, the selected dose is determined based on the number of subjects previously treated in the clinical trial and the actual probabilities of toxic outcomes and response outcomes among the subjects previously treated. In some embodiments, the subject is part of a cohort of subjects and all subjects in the cohort are administered the therapeutic agent at the same dose level.

In some embodiments, once the instructions, such as those containing or presented in a dose recommendation decision table, are prepared or obtained as described above, each cohort is treated at the most desirable acceptable dose. In some aspects, untried dose levels are not skipped. In some instances, if no dose level is acceptable based on combined futility and safety monitoring, the trial is stopped. In some cases, if no early stopping criteria are met, the trial is stopped once it reaches the maximum sample size. In some embodiments, the maximum sample size is within a range from about 1 to about 100, about 3 to about 60, or about 6 to about 30. In some aspects, the pre-specified maximum is or is about 6, 9, 12, 15, 18, 21, 24, 27, 30, 40, 50, 75, or 100. In some aspects, at the end of the trial, the most desirable acceptable dose based on combined safety and efficacy utility is selected.

### IV. Determination of Optimal Dose

Provided in some embodiments are methods for determining or selecting an optimal dose, such as to be used in a Phase II clinical trial based on data from a Phase I clinical trial, such as one carried out as described above. In some embodiments, when the number of subjects enrolled reaches the pre-specified maximum sample size, the trial is terminated and the best dose is selected. In some embodiments, the pre-specified maximum is within a range from about 1 to about 100, about 3 to about 60, or about 6 to about 30. In some aspects, the pre-specified maximum is or is about 6, 9, 12, 15, 18, 21, 24, 27, or 30.

In some embodiments, the method further includes identifying the optimal dose level. In some aspects, the model uses the function of safety and efficacy to choose the optimal dose. In some embodiments, depending on the clinical situation, a different metric could be used to make the final optimal dose selection. In some cases, the optimal dose level is associated with the highest probability that pᵢ is less than p_{T} and qᵢ is less than q_{E}. Thus, in some embodiments, the dose with the highest (or largest) probability Pr(pi<p_{T}, qi>q_{E}+δ|data) is selected as the optimal dose. In some such embodiments, δ is the expected increment over the minimum efficacy rate q_{E} for the therapy.

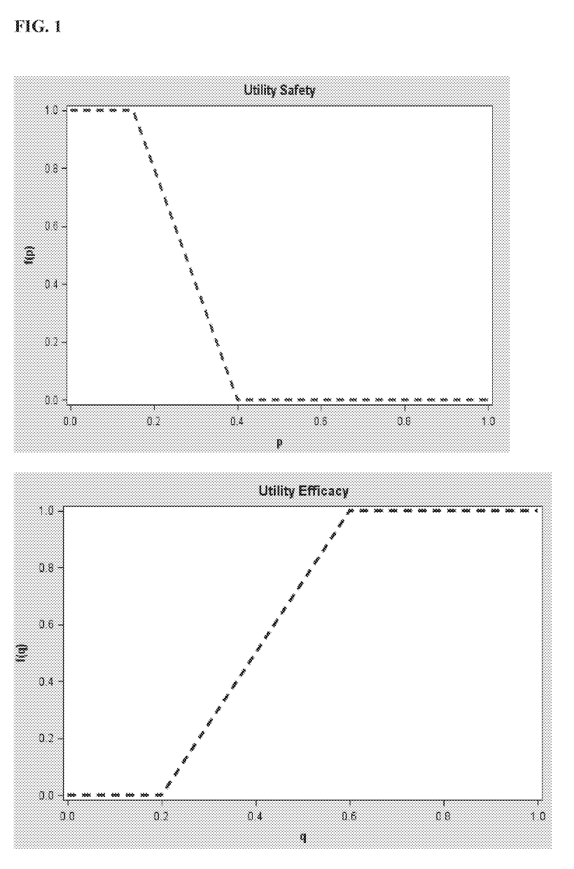

In some embodiments, the utility function for safety and efficacy can be constructed based on maximally tolerable safety and minimally acceptable efficacy parameters through discussions with the clinician or clinical team. For example, FIG. 1 (top) shows the utility function for safety. In some aspects, safety utility is defined as 1 if the DLT rate is less than or equal to 20%. In some cases, the safety utility is defined as 0 if the DLT rate is greater than 40%. In some instances, between a 20% and 40% DLT rate, safety decreases linearly as the DLT rate increases. In some embodiments, the safety utility function is constructed by taking into account the assumed overall safety profile for a dose level since some expected but toxic adverse events may not be part of the DLT in certain trials, such as adoptive cell therapy trials.

In some embodiments, the utility function for efficacy is as shown in FIG. 1 (bottom). In some cases, the efficacy utility function is defined as 0 if the response rate is less than 20%. In some aspects, the efficacy utility function is defined as 1 if the response rate is above 60%. In some embodiments,

In some embodiments, the utility score assesses all available doses by incorporating both their toxicity and efficacy rates, which can be determined prior to the trial. Thus, in some embodiments, the optimal dose level is selected based on the joint utility of safety and efficacy. In some cases, the optimal dose level is selected based on a combined utility function determined from both safety and efficacy utility functions:

u(p,a)=ƒ₁(p)ƒ₂(q)

where f₁(p) is decreasing with p, and f₂(q) is increasing with q.

In some embodiments, both f1(⋅) and f2(⋅) are truncated linear functions given by

$${f}_{1} ( p ) = \{ \begin{matrix}1 , & p ∈ ( 0 , {p}_{i}^{*} ] , \\ 1 - \frac{p - {p}_{1}^{*}}{{p}_{2}^{*} - {p}_{1}^{*}} , & p ∈ ( {p}_{1}^{*} , {p}_{2}^{*} ) , \\ 0 , & p ∈ [ {p}_{2}^{*} , 1 ) ,\end{matrix} and \\ {f}_{2} ( q ) = \{ \begin{matrix}0 , & q ∈ ( 0 , {q}_{i}^{*} ] , \\ \frac{q - {q}_{1}^{*}}{{q}_{2}^{*} - {q}_{1}^{*}} , & q ∈ ( {q}_{1}^{*} , {q}_{2}^{*} ) , \\ 1 , & q ∈ [ {q}_{2}^{*} , 1 ) ,\end{matrix}$$

In some aspects, there will be samples from the posterior distribution for safety and efficacy at every dose level. In some cases, for each of those samples from the posterior distribution, each dose has a corresponding utility score for safety and for efficacy, and they are multiplied to create a distribution of the dose level overall utility score.

For example, in some cases, for each dose i, a numerical approximation approach can be used to compute the posterior expected utility, E[U(pᵢ, qᵢ)|D]. In some instances, a total of T random samples are generated from the posterior distributions. In some embodiments, for each sample t, p^{t}=(p^{t}₁, . . . , p^{t}_{d}) and q^{t}=(q^{t}₁, . . . , q^{t}_{d}) are generated. In some aspects, the isotonic transformation is performed (Ji et al., 2007; 2010) on p^{t} to obtain \hat{ }p^{t}=(p\hat{ }^{t}, . . . , p\hat{ }^{t}_{d}) where p\hat{ }ᵢ^{t}≤p\hat{ }^{t}ⱼ if i<j. In some cases, this may ensure that p^{t} is non-decreasing. In some aspects, for each dose i, based on the samples qᵢ^{t} and p\hat{ }ᵢ^{t}, a corresponding utility score U^{t}(p\hat{ }ᵢ^{t}, pᵢ^{t}) is calculated according to above. In some such aspects, the estimated posterior expected utility is given by:

$$\hat{E} [ U ( {p}_{i} , {q}_{i} ) \vert D ] = \frac{1}{T} {\sum }_{t = 1}^{T} {U}^{t} ( {\hat{p}}_{i}^{t} , {q}_{i}^{t} )$$

In some embodiments, a monotonicity constraint is placed on the toxicity probability, i.e., p₁≤p₂≤p_{d}. In some aspects, the dose level with the largest expected posterior utility, such as according to the following formula, is selected:

î=argmaxᵢE[U(pᵢ,qᵢ)| [symbol] ]

In some instances, pi and qi are sensible estimates from the isotonically transformed posterior mean.

In some embodiments, the therapeutic agent is one for which the response outcome can be assessed within the timeframe in which the toxicity outcome is assessed. In some embodiments, the therapeutic agent comprises an adoptive cell therapy. In some embodiments, the adoptive cell therapy comprises a cell (e.g. T cell) engineered with a chimeric antigen receptor (CAR).

### V. Therapeutic Outcomes

In the provided methods, one or more therapeutic outcomes or events associated with toxicity (toxic outcome) and one or more therapeutic outcomes or events associated with efficacy (response outcome) of the therapeutic agent is assessed and dosing decisions are made in accord with the provided methods. In some embodiments, the provided methods determine dosing options for a therapeutic agent in which the therapy is known to work fast and/or in which the response outcome occurs rapidly, e.g. a therapeutic agent that is fast acting. In some cases, such therapeutic agents include agents that are specific or substantially specific for a particular disease or condition. In some embodiments, such therapeutic agents include, for example, small molecule drugs, gene therapies, molecularly targeted agents, immunotherapies and/or cell-based therapies. Thus, unlike other therapeutic agents that may be non-specific, such as cytotoxic or chemotherapeutic agents, the ability to achieve a response outcome faster or more rapidly means that the response outcome can be determined in a time frame that is the same or that is similar to the time frame at which a toxic outcome may occur. In some embodiments, the information about toxic outcome and response outcome can be jointly assessed in a subject, such as assessed in parallel or at around the same time or substantially the same time, and used to inform the dosing decisions or adaptive treatments of subjects.

In some embodiments, the toxic outcome and response outcome are monitored at a time at which a toxicity outcome and a response outcome are present. The particular time at which such outcome may be present will depend on the particular therapeutic agent and is known to a skilled artisan, such as a physician or clinician, or is within the level of such a skilled artisan to determine. In some embodiments, the time at which a toxic outcome or response outcome is assessed is within or within about a period of time in which a symptom of toxicity or efficacy is detectable in a subject or at such time in which an adverse outcome associated with non-response or toxicity is not detectable in the subject. In some embodiments, the time period is near or substantially near to when the toxic outcome and/or response outcome has peaked in the subject.

In some embodiments, the toxic outcome or response outcome can be assessed in the subject at a time that is within or about within 120 days after initiation of the first dose of the therapeutic agent to the subject, within or within about 90 days after initiation of the first dose, within or within about 60 days after initiation of the first dose of the therapeutic agent or within or within about 30 days after initiation of the first dose to a subject. In some embodiments, the toxic outcome or response can be assessed in the subject within or within about 6 days, 12 days, 16 days, 20 days, 24 days, 28 days, 32 days, 36 days, 40 days, 44 days, 48 days, 52 days, 56 days, 60 days, 64 days, 68 days, 72 days, 76 days, 80 days, 84 days, 88 days, 92 days, 96 days or 100 days after initiation of the first dose to a subject.

In some embodiments, the toxic outcome or response outcome is present or can be assessed or monitored at such time period where only a single dose of the therapeutic agent is administered. In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the first dose is a single or continuous administration of the specified number of cells, given or initiated at a single point in time. In some contexts, however, the first dose is administered in multiple injections or infusions over a period of no more than three days, such as once a day for three days or for two days or by multiple infusions over a single day period.

The term “split dose” refers to a dose that is split so that it is administered over more than one day. This type of dosing is encompassed by the present methods and is considered to be a single dose.

As used herein, “first dose” is used to describe the timing of a given dose, which, in some cases can be the only dose or can be followed by one or more repeat or additional doses. The term does not necessarily imply that the subject has never before received a dose of a therapeutic agent even that the subject has not before received a dose of the same or substantially the same therapeutic agent.

In some embodiments, the toxic outcome or response outcome is present and/or can be assessed or monitored at such time period that is after a first cycle of administration of the therapeutic agent, after a second cycle of administration of the therapeutic agent, after a third cycle of administration of the therapeutic agent, or after a fourth cycle of administration of the therapeutic agent. In some embodiments, a cycle of administration can be a repeated schedule of a dosing regimen that is repeated over successive administrations. In some embodiments, a schedule of administration can be daily, every other day, or once a week for one week, two weeks, three weeks or four weeks (e.g. 28 days). In some embodiments, a cycle of administration can be tailored to add periods of discontinued treatment in order to provide a rest period from exposure to the agent. In some embodiments, the length of time for the discontinuation of treatment can be for a predetermined time or can be empirically determined depending on how the patient is responding or depending on observed side effects. For example, the treatment can be discontinued for one week, two weeks, one month or several months.

In some embodiments, the toxic outcome and response outcome can be assessed by monitoring one or more symptoms or events associated with a toxic outcome and one or more symptoms or events associated with a response outcome. In some embodiments, the disease or condition is a tumor or cancer.

A. Toxic Outcome

In some embodiments, a toxic outcome in a subject to administration of a therapeutic agent (e.g. CAR T-cells) can be assessed or monitored. In some embodiments, the toxic outcome is or is associated with the presence of a toxic event, such as cytokine release syndrome (CRS), severe CRS (sCRS), macrophage activation syndrome, tumor lysis syndrome, fever of at least at or about 38 degrees Celsius for three or more days and a plasma level of CRP of at least at or about 20 mg/dL, neurotoxicity and/or neurotoxicity. In some embodiments, the toxic outcome is a sign, or symptom, particular signs, and symptoms and/or quantities or degrees thereof which presence or absence may specify a particular extent, severity or level of toxicity in a subject. It is within the level of a skilled artisan to specify or determine a particular sign, symptom and/or quantities or degrees thereof that are related to an undesired toxic outcome of a therapeutic agent (e.g. CAR-T cells).

In some embodiments, the toxic outcome is an indicator associated with the toxic event. In some embodiments, the toxic outcome is the presence or absence of one or more biomarkers or the presence of absence of a level of one or more biomarkers. In some embodiments, the biomarker is molecule present in the serum or other bodily fluid or tissue indicative of cytokine-release syndrome (CRS), severe CRS or CRS-related outcomes. In some embodiments, the biomarker is a molecule present in the serum or other bodily fluid or tissue indicative of neurotoxicity or severe neurotoxicity.

In some embodiments, the subject exhibits toxicity or a toxic outcome if a toxic event, such as CRS-related outcomes, e.g. if a serum level of an indicator of CRS or other biochemical indicator of the toxicity is more than at or about 10 times, more than at or about 15 times, more than at or about 20 times, more than at or about 25 times, more than at or about 50 times, more than at or about 75 times, more than at or about 100 times, more than at or about 125 times, more than at or about 150 times, more than at or about 200 times, or more than at or about 250 times the baseline or pre-treatment level, such as the serum level of the indicator immediately prior to administration of the first dose of the therapeutic agent.

Exemplary signs or symptoms associated with CRS include fever, rigors, chills, hypotension, dyspnea, acute respiratory distress syndrome (ARDS), encephalopathy, ALT/AST elevation, renal failure, cardiac disorders, hypoxia, neurologic disturbances, and death. Neurological complications include delirium, seizure-like activity, confusion, word-finding difficulty, aphasia, and/or becoming obtunded. Other CRS-related signs or outcomes include fatigue, nausea, headache, seizure, tachycardia, myalgias, rash, acute vascular leak syndrome, liver function impairment, and renal failure. In some aspects, CRS is associated with an increase in one or more factors such as serum-ferritin, d-dimer, aminotransferases, lactate dehydrogenase and triglycerides, or with hypofibrinogenemia or hepatosplenomegaly.

In some embodiments, signs or symptoms associated with CRS include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines (e.g. IFNγ or IL-6); and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures).

In some embodiments, the presence of one or more biomarkers is indicative of the grade of, severity or extent of a toxic event, such as CRS or neurotoxicity. In some embodiments, the toxic outcome is a particular grade, severity or extent of a toxic event, such as a particular grade, severity or extent of CRS or neurotoxicity. In some embodiments, the presence of a toxic event about a certain grade, severity or extent can be a dose-limiting toxicity. In some embodiments, the absence of a toxic event or the presence of a toxic event below a certain grade, severity or extent can indicate the absence of a dose-limiting toxicity.

CRS criteria that appear to correlate with the onset of CRS to predict which patients are more likely to be at risk for developing sCRS have been developed (see Davilla et al. Science translational medicine. 2014; 6(224):224ra25). Factors include fevers, hypoxia, hypotension, neurologic changes, and elevated serum levels of inflammatory cytokines whose treatment-induced elevation can correlate well with both pretreatment tumor burden and sCRS symptoms. Other guidelines on the diagnosis and management of CRS are known (see e.g., Lee et al, Blood. 2014; 124(2):188-95). In some embodiments, the criteria reflective of CRS grade are those detailed in Table 2 below.

TABLE 2
Exemplary Grading Criteria for CRS

| Grade | Description of Symptoms |
|---|---|
| 1 | Not life-threatening, require only symptomatic treatment |
| Mild | such as antipyretics and anti-emetics (e.g., fever, nausea, |
| | fatigue, headache, myalgias, malaise) |
| 2 | Require and respond to moderate intervention: |
| Moderate | Oxygen requirement < 40%, or |
| | Hypotension responsive to fluids or low dose of a single |
| | vasopressor, or |
| | Grade 2 organ toxicity (by CTCAE v4.0) |
| 3 | Require and respond to aggressive intervention: |
| Severe | Oxygen requirement ≥ 40%, or |
| | Hypotension requiring high dose of a single vasopressor |
| | (e.g., norepinephrine ≥ 20 μg/kg/min, |
| | dopamine ≥ 10 μg/kg/min, |
| | phenylephrine ≥ 200 μg/kg/min, or |
| | epinephrine ≥ 10 μg/kg/min), or |
| | Hypotension requiring multiple vasopressors |
| | (e.g., vasopressin + one of the above agents, |
| | or combination vasopressors equivalent |
| | to ≥20 μg/kg/min norepinephrine), or |
| | Grade 3 organ toxicity or Grade 4 transaminitis |
| | (by CTCAE v4.0) |
| 4 | Life-threatening: |
| Life- | Requirement for ventilator support, or |
| threatening | Grade 4 organ toxicity (excluding transaminitis) |
| 5 | Death |
| Fatal | |

In some embodiments, the toxic outcome is severe CRS. In some embodiments, the toxic outcome is the absence of severe CRS (e.g. moderate or mild CRS). In some embodiments, severe CRS includes CRS with a grade of 3 or greater, such as set forth in Table 2. In some embodiments, the level of the toxic outcome, e.g. the CRS-related outcome, e.g. the serum level of an indicator of CRS, is measured by ELISA. In some embodiments, fever and/or levels of CRP can be measured. In some embodiments, subjects with a fever and a CRP≥15 mg/dL may be considered high-risk for developing severe CRS.

In some aspects, the toxic outcome is or is associated with neurotoxicity. In some embodiments, signs or symptoms associated with a clinical risk of neurotoxicity include confusion, delirium, expressive aphasia, obtundation, myoclonus, lethargy, altered mental status, convulsions, seizure-like activity, seizures (optionally as confirmed by electroencephalogram [EEG]), elevated levels of beta amyloid (Aβ), elevated levels of glutamate, and elevated levels of oxygen radicals. In some embodiments, neurotoxicity is graded based on severity (e.g., using a Grade 1-5 scale (see, e.g., Guido Cavaletti & Paola Marmiroli Nature Reviews Neurology 6, 657-666 (December 2010); National Cancer Institute—Common Toxicity Criteria version 4.03 (NCI-CTCAE v4.03). In some embodiments, severe neurotoxicity includes neurotoxicity with a grade of 3 or greater, such as set forth in Table 3.

TABLE 3
Exemplary Grading Criteria for neurotoxicity

| Grade | Description of Symptoms |
|---|---|
| 1 | Mild or asymptomatic symptoms |
| Asymptomatic | |
| or Mild | |
| 2 | Presence of symptoms that limit instrumental activities |
| Moderate | of daily living (ADL), such as preparing meals, |
| | shopping for groceries or clothes, using the telephone, |
| | managing money |
| 3 | Presence of symptoms that limit self-care ADL, such |
| Severe | as bathing, dressing and undressing, feeding self, |
| | using the toilet, taking medications |
| 4 | Symptoms that are life-threatening, requiring urgent |
| Life-threatening | intervention |
| 5 | Death |
| Fatal | |

In some embodiments, the toxic outcome is a dose-limiting toxicity. In some embodiments, the toxic outcome is the absence of a dose-limiting toxicity. In some embodiments, a dose-limiting toxicity (DLT) is defined as any grade 3 or higher toxicity as assessed by any known or published guidelines for assessing the particular toxicity, such as any described above and including the National Cancer Institute (NCI) Common Terminology Criteria for Adverse Events (CTCAE) version 4.0.

B. Response Outcome

In some embodiments, a response outcome in a subject to administration of a therapeutic agent can be monitored or assessed. In some embodiments, the response outcome is no response. In some embodiments, the response outcome is a partial response. In some embodiments, the response outcome is a complete response (CR). In some embodiments, response outcome is assessed by monitoring the disease burden in the subject. In some embodiments, the presence of no response, a partial response or a clinical or complete response can be assessed.

In some embodiments, a partial response or complete response is one in which the therapeutic agent reduces or prevents the expansion or burden of the disease or condition in the subject. For example, where the disease or condition is a tumor, reduced disease burden exists or is present if there is a reduction in the tumor size, bulk, metastasis, percentage of blasts in the bone marrow or molecularly detectable cancer and/or an improvement prognosis or survival or other symptom associated with tumor burden compared to prior to treatment with the therapeutic agent (e.g. CAR T cells).

In some embodiments, the disease or condition is a tumor and a reduction in disease burden is a reduction in tumor size. In some embodiments, the disease burden reduction is indicated by a reduction in one or more factors, such as load or number of disease cells in the subject or fluid or organ or tissue thereof, the mass or volume of a tumor, or the degree or extent of metastases. In some embodiments, disease burden, e.g. tumor burden, can be assessed or monitored for the extent of morphological disease and/or minimal residual disease.

In some embodiments, the burden of a disease or condition in the subject is detected, assessed, or measured. Disease burden may be detected in some aspects by detecting the total number of disease or disease-associated cells, e.g., tumor cells, in the subject, or in an organ, tissue, or bodily fluid of the subject, such as blood or serum. In some embodiments, disease burden, e.g. tumor burden, is assessed by measuring the mass of a solid tumor and/or the number or extent of metastases. In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of disease or condition burden is specified.

In some embodiments, disease burden can encompass a total number of cells of the disease in the subject or in an organ, tissue, or bodily fluid of the subject, such as the organ or tissue of the tumor or another location, e.g., which would indicate metastasis. For example, tumor cells may be detected and/or quantified in the blood or bone marrow in the context of certain hematological malignancies. Disease burden can include, in some embodiments, the mass of a tumor, the number or extent of metastases and/or the percentage of blast cells present in the bone marrow.

In some embodiments, a subject has leukemia. The extent of disease burden can be determined by assessment of residual leukemia in blood or bone marrow.

In some embodiments, a response outcome exists if there is a reduction in the percent of blasts in the bone marrow compared to the percent of blasts in the bone marrow prior to treatment with the therapeutic agent. In some embodiments, reduction of disease burden exists if there is a decrease or reduction of at least or at least about 20%, 30%, 40%, 50%, 60%, 70%, 80%, 90%, 95% or more in the number or percentage of blasts in the bone marrow compared to the number or percent of blasts in the bone marrow prior to treatment.

In some embodiments, the subject exhibits a response if the subject does not exhibit morphologic disease (non-morphological disease) or does not exhibit substantial morphologic disease. In some embodiments, a subject exhibits morphologic disease if there are greater than or equal to 5% blasts in the bone marrow, for example, as detected by light microscopy. In some embodiments, a subject exhibits complete or clinical remission if there are less than 5% blasts in the bone marrow.

In some embodiments, a subject exhibits reduced or decreased disease burden if they exhibited morphological disease prior to treatment and exhibit complete remission (e.g., fewer than 5% blasts in bone marrow) with or without molecular disease (e.g., minimum residual disease (MRD) that is molecularly detectable, e.g., as detected by flow cytometry or quantitative PCR) after treatment. In some embodiments, a subject exhibits reduced or decreased disease burden if they exhibited molecular disease prior to treatment and do not exhibit molecular disease after treatment.

In some embodiments, a subject may exhibit complete remission, but a small proportion of morphologically undetectable (by light microscopy techniques) residual leukemic cells are present. A subject is said to exhibit minimum residual disease (MRD) if the subject exhibits less than 5% blasts in the bone marrow and exhibits molecularly detectable cancer. In some embodiments, molecularly detectable cancer can be assessed using any of a variety of molecular techniques that permit sensitive detection of a small number of cells. In some aspects, such techniques include PCR assays, which can determine unique Ig/T-cell receptor gene rearrangements or fusion transcripts produced by chromosome translocations. In some embodiments, flow cytometry can be used to identify cancer cell based on leukemia-specific immunophenotypes. In some embodiments, molecular detection of cancer can detect as few as 1 leukemia or blast cell in 100,000 normal cells or 1 leukemia or blast cell in 10,000 normal cells. In some embodiments, a subject exhibits MRD that is molecularly detectable if at least or greater than 1 leukemia cell in 100,000 cells is detected, such as by PCR or flow cytometry.

In some embodiments, the disease burden of a subject is molecularly undetectable or MRD⁻, such that, in some cases, no leukemia cells are able to be detected in the subject using PCR or flow cytometry techniques.

In some embodiments the response outcome is the absence of a CR or the presence of a complete response in which the subject achieves or exhibits minimal residual disease or molecular detectable disease status. In some embodiments, the response outcome is the presence of a CR with molecularly detectable disease or the presence of a CR without molecularly detectable disease. In some embodiments, subjects are assessed for disease burden using methods as described herein, such as methods that assess blasts in bone marrow or molecular disease by flow cytometry or qPCR methods.

### VI. Administering a Therapeutic Agent

In some embodiments, the provided methods can be used for determining dosing actions or adapting dosing regimens for administering a therapeutic agent. In some embodiments, the therapeutic agent is specific or substantially specific for or acts preferentially on or is targeted against a disease or condition. In some embodiments, the therapeutic agent is one who, compared to conventional chemotherapeutic or cytotoxic agents, exhibits a fast or rapid response. In some embodiments, the therapeutic agent is a molecularly targeted agent, an immunotherapy and/or a cell therapy. In some embodiments, the therapeutic agent is a small molecule, a nucleic acid, a peptide or is a polypeptide or protein. In some embodiments, the therapeutic agent is a targeted antibody therapy, such as a bispecific antibody therapy, including an anti-CD3 bispecific antibody therapy (e.g. Blinatumomab). In some embodiments, the therapeutic agent is a checkpoint inhibitor, such as an anti-checkpoint antibody, including an anti-PD-L1 antibody, anti-PD-1 antibody, or anti-CTLA-4 antibody. In some embodiments, the therapeutic agent is an adoptive cell therapy, such as any T cell therapy, for example, a tumor infiltrating lymphocytic (TIL) therapy, a transgenic TCR therapy or a chimeric antigen receptor (CAR)-expressing T cell therapy.

A. Adoptive Cell Therapy

I. Cells

The cells generally are eukaryotic cells, such as mammalian cells, and typically are human cells, e.g., those derived from human subjects and engineered, for example, to express the recombinant receptors. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

2. Vectors and Methods for Genetic Engineering

In some embodiments, the cells expressing a recombinant receptor are produced by the genetically engineered cells expressing recombinant receptors. The genetic engineering generally involves introduction of a nucleic acid encoding the recombinant or engineered component into the cell, such as by retroviral transduction, transfection, or transformation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the subject to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell II: 223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some aspects, the cells further are engineered to promote expression of cytokines or other factors. Various methods for the introduction of genetically engineered components, e.g., antigen receptors, e.g., CARs, are well known and may be used with the provided methods and compositions. Exemplary methods include those for transfer of nucleic acids encoding the receptors, including via viral, e.g., retroviral or lentiviral, transduction, transposons, and electroporation.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

3. Preparation of Cells for Engineering

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some aspects, the cells of the second dose are derived from the same apheresis product as the cells of the first dose. In some embodiments, the cells of multiple doses, e.g., first, second, third, and so forth, are derived from the same apheresis product.

In other embodiments, the cells of the second (or other subsequent) dose are derived from an apheresis product that is distinct from that from which the cells of the first (or other prior) dose are derived.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using CD3/CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood.1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻ CD8⁺ and/or CD62L⁺ CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or subpopulation, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L+ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher© Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotech, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotic), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood.1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood.1:72-82, and/or Wang et al. (2012) J Immunother. 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

4. Recombinant Receptors Expressed by the Cells

The cells generally express recombinant receptors. The receptors may include antigen receptors, such as functional non-TCR antigen receptors, including chimeric antigen receptors (CARs), and other antigen-binding receptors such as transgenic T cell receptors (TCRs). The receptors may also include other chimeric receptors, such as receptors binding to particular ligands and having transmembrane and/or intracellular signaling domains similar to those present in a CAR.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also International Patent Publication No.: WO2014031687, U.S. Pat. Nos. 8,339,645, 7,446,179, 7,446,190, and 8,389,282, and U.S. patent application Publication No. US 2013/0149337. Among the chimeric receptors are chimeric antigen receptors (CARs). The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment.

In some embodiments, the binding domain(s), e.g., the antibody, e.g., antibody fragment, portion of the recombinant receptor further includes at least a portion of an immunoglobulin constant region, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers, e.g., hinge regions, include those described in international patent application publication number WO2014031687. In some examples, the spacer is or is about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153, international patent application publication number WO2014031687, U.S. Pat. No. 8,822,647 or published app. No. US2014/0271635. In some embodiments, the constant region or portion is of a human IgG, such as IgG4 or IgG1.

This antigen recognition domain generally is linked to one or more intracellular signaling components, such as signaling components that mimic activation through an antigen receptor complex, such as a TCR complex, and/or signal via another cell surface receptor. The signal may be immunostimulatory and/or costimulatory in some embodiments. In some embodiments, it may be suppressive, e.g., immunosuppressive. Thus, in some embodiments, the antigen-binding component (e.g., antibody) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD 16, CD22, CD33, CD37, CD64, CD80, CD86, CD 134, CD137, CD 154 and/or transmembrane regions containing functional variants thereof such as those retaining a substantial portion of the structural, e.g., transmembrane, properties thereof. In some embodiments, the transmembrane domain is a transmembrane domain derived from CD4, CD28, or CD8, e.g., CD8alpha, or functional variant thereof. The transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR or other chimeric receptor includes a chimeric molecule between CD3-zeta (CD3-ζ) or Fc receptor γ and CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence derived from a signaling molecule or domain that promotes primary activation of a TCR complex in a natural setting. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from the CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the CAR includes a signaling domain and/or transmembrane portion of a costimulatory receptor, such as CD28, 4-1BB, OX40, DAP10, and ICOS. In some aspects, the same CAR includes both the activating and costimulatory components.

In some embodiments, the activating domain is included within one CAR, whereas the costimulatory component is provided by another CAR recognizing another antigen, present on the same cell. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the intracellular signaling component of the recombinant receptor, such as CAR, comprises a CD3 zeta intracellular domain and a costimulatory signaling region. In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the CAR or other antigen receptor further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

In some embodiments, the chimeric antigen receptor includes an extracellular portion containing an antigen-binding domain, such as an antibody or antigen-binding antibody fragment, such as an scFv or Fv. In some aspects, the chimeric antigen receptor includes an extracellular portion containing the antibody or fragment and an intracellular signaling domain. In some embodiments, the antibody or fragment includes an scFv and the intracellular domain contains an ITAM. In some aspects, the intracellular signaling domain includes a signaling domain of a zeta chain of a CD3-zeta (CD3) chain. In some embodiments, the chimeric antigen receptor includes a transmembrane domain linking the extracellular domain and the intracellular signaling domain. In some aspects, the transmembrane domain contains a transmembrane portion of CD28. In some embodiments, the chimeric antigen receptor contains an intracellular domain of a T cell costimulatory molecule. The extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

For example, in some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof In some such embodiments, the receptor further includes a spacer containing a portion of an Ig molecule, such as a human Ig molecule, such as an Ig hinge, e.g. an IgG4 hinge, such as a hinge-only spacer.

In some embodiments, the chimeric antigen receptor contains an intracellular domain of a T cell costimulatory molecule. In some aspects, the T cell costimulatory molecule is CD28 or 41BB.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374).

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

5. Compositions and Formulations

In some embodiments, the cells, such as cells genetically engineered with a recombinant receptor (e.g. CAR-T cells) are provided as compositions, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

The cells and compositions may be administered using standard administration techniques, formulations, and/or devices. Administration of the cells can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

6. Methods of Administration

In some embodiments, the provided methods generally involve administering doses of cells expressing recombinant molecules such as recombinant receptors, such as CARs, other chimeric receptors, or other antigen receptors, such as transgenic TCRs, to subjects having a disease or condition, such as a disease or condition a component of which is specifically recognized by and/or treated by the recombinant molecules, e.g., receptors. The administrations generally effect an improvement in one or more symptoms of the disease or condition and/or treat or prevent the disease or condition or symptom thereof.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, HPV, and parasitic disease, and autoimmune and inflammatory diseases. In some embodiments, the disease or condition is a tumor, cancer, malignancy, neoplasm, or other proliferative disease or disorder. Such diseases include but are not limited to leukemia, lymphoma, e.g., chronic lymphocytic leukemia (CLL), ALL, non-Hodgkin's lymphoma, acute myeloid leukemia, multiple myeloma, refractory follicular lymphoma, mantle cell lymphoma, indolent B cell lymphoma, B cell malignancies, cancers of the colon, lung, liver, breast, prostate, ovarian, skin, melanoma, bone, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, and/or mesothelioma.

In some embodiments, the disease or condition is a tumor and the subject has a large tumor burden prior to the administration of the first dose, such as a large solid tumor or a large number or bulk of disease-associated, e.g., tumor, cells. In some aspects, the subject has a high number of metastases and/or widespread localization of metastases. In some aspects, the tumor burden in the subject is low and the subject has few metastases. In some embodiments, the size or timing of the doses is determined by the initial disease burden in the subject. For example, whereas in some aspects the subject may be administered a relatively low number of cells in the first dose, in context of lower disease burden the dose may be higher.

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, the antigen associated with the disease or disorder is selected from the group consisting of orphan tyrosine kinase receptor ROR1, tEGFR, Her2, Ll-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, OEPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, and MAGE A3, CE7, Wilms Tumor 1 (WT-1), a cyclin, such as cyclin A1 (CCNA1), and/or biotinylated molecules, and/or molecules expressed by HIV, HCV, HBV or other pathogens.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply necessarily complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical or similar. In some embodiments, the second subject expresses the same HLA class or supertype as the first subject.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, intrathoracic, intracranial, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or other agent, such as a cytotoxic or therapeutic agent. Thus, the cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2 or other cytokine, for example, to enhance persistence.

In some embodiments, the methods comprise administration of a chemotherapeutic agent, e.g., a conditioning chemotherapeutic agent, for example, to reduce tumor burden prior to the dose administrations.

Once the cells are administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD 107a, IFNγ, IL-2, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

In certain embodiments, engineered cells are modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known in the art. See, for instance, Wadwa et al., J. Drug Targeting 3: 1 1 1 (1995), and U.S. Pat. No. 5,087,616.

7. Dosing

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells are administered in a single pharmaceutical composition.

In some embodiments, the cells are administered in a plurality of compositions, collectively containing the cells of a single dose.

Thus, one or more of the doses in some aspects may be administered as a split dose. For example, in some embodiments, the dose may be administered to the subject over 2 days or over 3 days. Exemplary methods for split dosing include administering 25% of the dose on the first day and administering the remaining 75% of the dose on the second day. In other embodiments 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, multiple doses are given, in some aspects using the same timing guidelines as those with respect to the timing between the first and second doses, e.g., by administering a first and multiple subsequent doses, with each subsequent dose given at a point in time that is greater than about 28 days after the administration of the first or prior dose.

In some embodiments, the dose contains a number of cells, number of recombinant receptor (e.g., CAR)-expressing cells, number of T cells, or number of peripheral blood mononuclear cells (PBMCs) in the range from about 10⁵ to about 10⁶ of such cells per kilogram body weight of the subject, and/or a number of such cells that is no more than about 10⁵ or about 10⁶ such cells per kilogram body weight of the subject. For example, in some embodiments, the first or subsequent dose includes less than or no more than at or about 1×10⁵, at or about 2×10⁵, at or about 5×10⁵, or at or about 1×10⁶ of such cells per kilogram body weight of the subject. In some embodiments, the first dose includes at or about 1×10⁵, at or about 2×10⁵, at or about 5×10⁵, or at or about 1×10⁶ of such cells per kilogram body weight of the subject, or a value within the range between any two of the foregoing values. In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some embodiments, for example, where the subject is a human, the dose includes fewer than about 1×10⁸ total recombinant receptor (e.g., CAR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values. In certain embodiments, the dose comprises fewer than about 1×10¹², about 1×10¹¹, about 1×10¹⁰, about 1×10⁹, about 1×10⁸, or about 1×10⁷, total recombinant receptor (e.g., CAR)-expressing cells. In particular embodiments, the dose includes between about 1×10⁶ to 1×10⁸, about 1×10⁹ to 1×10¹¹, about 1×10¹⁰ to 1×10¹² total recombinant receptor (e.g., CAR)-expressing cells, T cells, or peripheral blood mononuclear cells (PBMCs). In certain embodiments, the dose includes between about 1×10⁶ to 1×10⁷, about 1×10⁹ to 1×10¹¹ such cells, about 1×10¹⁰ to 1×10¹² total recombinant receptor (e.g., CAR)-expressing cells, T cells, or peripheral blood mononuclear cells (PBMCs).

In some embodiments, the dose contains fewer than about 1×10⁸ total recombinant receptor (e.g., CAR)-expressing cells, T cells, or peripheral blood mononuclear cells (PBMCs) cells per m² of the subject, e.g., in the range of about 1×10⁶ to 1×10⁸ such cells per m² of the subject, such as 2×10⁶, 5×10⁶, 1×10⁷, 5×10⁷, or 1×10⁸ such cells per m² of the subject, or the range between any two of the foregoing values.

In certain embodiments, the number of cells, recombinant receptor (e.g., CAR)-expressing cells, T cells, or peripheral blood mononuclear cells (PBMCs) in the dose is greater than about 1×10⁶ such cells per kilogram body weight of the subject, e.g., 2×10⁶, 3×10⁶, 5×10⁶, 1×10⁷, 5×10⁷, 1×10⁸, 1×10⁹, or 1×10¹⁰ such cells per kilogram of body weight and/or, 1×10⁸, or 1×10⁹, 1×10¹⁰ such cells per m² of the subject or total, or the range between any two of the foregoing values.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the size of the dose is determined by the burden of the disease or condition in the subject. For example, in some aspects, the number of cells administered in the dose is determined based on the tumor burden that is present in the subject immediately prior to administration of the initiation of the dose of cells. In some embodiments, the size of the first and/or subsequent dose is inversely correlated with disease burden. In some aspects, as in the context of a large disease burden, the subject is administered a low number of cells, for example less than about 1×10⁶ cells per kilogram of body weight of the subject. In other embodiments, as in the context of a lower disease burden, the subject is administered a larger number of cells, such as more than about 1×10⁶ cells per kilogram body weight of the subject.

### VII. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, a composition refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, a “subject” is a mammal, such as a human or other animal, and typically is human.

### VIII. Exemplary Embodiments

1. A method for providing a dose recommendation for a therapeutic agent in a clinical trial, comprising:

a) obtaining a matrix comprising one or more dosing actions associated with a combination of toxicity and efficacy probability intervals in a clinical trial;

b) determining a joint unit probability mass (UPM) for each combination of toxicity and efficacy probability intervals for one or more possible toxicity probabilities at current dose level i (pᵢ) and one or more possible efficacy probabilities at current dose level i (qᵢ);

c) identifying the combination of toxicity and efficacy intervals that has the highest joint UPM;

d) assigning the dosing action associated with the identified combination as a dose recommendation for each of the one or more possible toxicity and efficacy probabilities; and

e) producing or outputting instructions that specify the dose recommendations.

2. A computer implemented method for providing a dose recommendation for a therapeutic agent in a clinical trial, comprising:

at an electronic device having a processor and memory:
a) obtaining a matrix comprising one or more dosing actions associated with a combination of toxicity and efficacy probability intervals in a clinical trial;
b) determining, by the processor, a joint unit probability mass (UPM) for each combination of toxicity and efficacy probability intervals for one or more possible toxicity probabilities at current dose level i (pᵢ) and one or more possible efficacy probabilities at current dose level i (qᵢ);
c) identifying the combination of toxicity and efficacy intervals that has the highest joint UPM;
d) assigning the dosing action associated with the identified combination as a dose recommendation for each of the one or more possible toxicity and efficacy probabilities; and
e) producing or outputting instructions that specify the dose recommendations.

3. A method for providing a dose recommendation for a therapeutic agent, comprising:

a) obtaining a matrix comprising one or more dosing actions associated with a combination of toxicity and efficacy probability intervals;

b) determining a joint unit probability mass (UPM) for each combination of toxicity and efficacy probability intervals for one or more possible toxicity probabilities at current dose level i (pᵢ) and one or more possible efficacy probabilities at current dose level i (qᵢ);

c) identifying the combination of toxicity and efficacy intervals that has the highest joint UPM;

d) assigning the dosing action associated with the identified combination as a dose recommendation for each of the one or more possible toxicity and efficacy probabilities; and

e) producing or outputting instructions that specify the dose recommendations.

4. A method for providing a dose recommendation for a therapeutic agent, comprising:

a) identifying a combination of toxicity and efficacy intervals that has the highest joint unit probability mass (UPM) from a matrix, wherein the matrix comprises combinations of toxicity and efficacy probability intervals at current dose level i and one or more dosing actions associated with said toxicity and efficacy probabilities;

b) assigning the dosing action associated with the identified combination as a dose recommendation for each of the one or more possible toxicity and efficacy probabilities; and

c) producing or outputting instructions that specify the dose recommendations.

5. A computer implemented method for providing a dose recommendation for a therapeutic agent, comprising:

at an electronic device having a processor and memory:

a) obtaining a matrix comprising one or more dosing actions associated with a combination of toxicity and efficacy probability intervals;

b) determining, by the processor, a joint unit probability mass (UPM) for each combination of toxicity and efficacy probability intervals for one or more possible toxicity probabilities at current dose level i (pᵢ) and one or more possible efficacy probabilities at current dose level i (qᵢ);

c) identifying the combination of toxicity and efficacy intervals that has the highest joint UPM;

d) assigning the dosing action associated with the identified combination as a dose recommendation for each of the one or more possible toxicity and efficacy probabilities; and

e) producing or outputting instructions that specify the dose recommendations.

6. A computer implemented method for providing a dose recommendation for a therapeutic agent, comprising:

at an electronic device having a processor and memory:

a) identifying a combination of toxicity and efficacy intervals that has the highest joint unit probability mass (UPM) from a matrix, wherein the matrix comprises combinations of toxicity and efficacy probability intervals at current dose level i and one or more dosing actions associated with said toxicity and efficacy probabilities;

b) assigning the dosing action associated with the identified combination as a dose recommendation for each of the one or more possible toxicity and efficacy probabilities; and

c) producing or outputting instructions that specify the dose recommendations.

7. The method of any of embodiments 1-6, wherein the therapeutic agent is administered in a clinical trial.

8. The method of any of embodiments 1-7, wherein the matrix is created by:
i) designating two or more toxicity probability intervals of the therapeutic agent and two or more efficacy probability intervals of the therapeutic agent; and
ii) assigning a dosing action to each combination of toxicity and efficacy probability intervals.

9. The method of any of embodiments 1-8, wherein the matrix comprises two toxicity probability intervals.

10. The method of any of embodiments 1-8, wherein the matrix comprises three toxicity probability intervals.

11. The method of any of embodiments 1-8, wherein the matrix comprises four toxicity probability intervals.

12. The method of any of embodiments 1-11, wherein the matrix comprises two efficacy probability intervals.

13. The method of any of embodiments 1-11, wherein the matrix comprises three efficacy probability intervals.

14. The method of any of embodiments 1-11, wherein the matrix comprises four efficacy probability intervals.

15. The method of any of embodiments 1-14, wherein the dosing action and/or dose recommendation is escalate (E) to dose level i+1.

16. The method of any of embodiments 1-14, wherein the dosing action and/or dose recommendation is stay (S) at dose level i.

17. The method of any of embodiments 1-14, wherein the dosing action and/or dose recommendation is de-escalate (D) to dose level i−1.

18. The method of any of embodiments 1-17, further comprising prior to step e), altering the dose recommendation to:

a) de-escalate and not return to current dose if the probability that pᵢ is greater than the maximum acceptable toxicity probability (p_{T}) exceeds 0.95;

b) de-escalate and not return to current dose if the probability that qᵢ is less than the minimum acceptable efficacy probability (q_{E}) exceeds 0.7; or

c) escalate and not return to current dose if the probability that qᵢ is less than q_{E} exceeds 0.7.

19. The method of any of embodiments 1-18, further comprising prior to step e), altering the dose recommendation to:

(a) de-escalate and not return to current dose if pᵢ is greater than p_{T};

(b) de-escalate and not return to current dose if qᵢ is less than q_{E}; or

(c) escalate and not return to current dose if qᵢ is less than q_{E}.

20. The method of any of embodiments 1-18, wherein prior to the step of producing or outputting instructions, the method further comprises altering the dose recommendation to:

(a) de-escalate and not return to current dose if pᵢ is greater than p_{T};

(b) de-escalate and not return to current dose if qᵢ is less than q_{E}; or

(c) escalate and not return to current dose if qᵢ is less than q_{E}.

21. The method of any of embodiments 1-14 and 18-20, wherein the dose recommendation is de-escalate and do not return to the current dose level or any higher dose level (DU).

22. The method of any of embodiments 1-14 and 18-20, wherein the dose recommendation is de-escalate and do not return to the current dose level or any higher dose level (DEU).

23. The method of any of embodiments 1-14 and 18-20, wherein the dose recommendation is escalate and do not return to the current dose level or any lower dose level (EEU).

24. The method of any of embodiments 1-23, comprising prior to step a) obtaining the maximum acceptable toxicity probability (p_{T}) and minimum acceptable efficacy probability (q_{E}) of the therapeutic agent.

25. The method of any of embodiments 1-24, wherein each toxicity probability interval is defined by a start value a and an end value b and each efficacy probability interval is defined by a start value c and an end value d.

26. The method of embodiment 25, wherein each combination of toxicity and efficacy probability intervals is defined as (a, b)×(c, d).

27. The method of any of embodiments 1-26, wherein the matrix comprises a two-way grid.

28. The method of embodiment 27, wherein the one or more dosing actions associated with the combination of toxicity and efficacy probability intervals are displayed in the two-way grid.

29. The method of any of embodiments 1-28, wherein the determining the joint UPM for each combination of toxicity and probability intervals comprises:

a) determining the probability that pᵢ and qᵢ are contained within the combination of toxicity and probability intervals;

b) dividing by the product of the toxicity probability interval length and the efficacy probability interval length.

30. The method of any of embodiments 1-29, wherein the joint UPM (JUPM) is determined as:

$$JUPM \begin{matrix}( c , d ) \\ ( a , b )\end{matrix} ≡ \frac{Pr ( {p}_{i} ∈ ( a , b ) , {q}_{i} ∈ ( c , d ) \vert 𝒟 )}{( b - a ) \times ( d - c )} , \\ 0 < a < b < 1 , 0 < c < d < 1. \qquad ( 1 )$$

31. The method of any of embodiments 1-30, wherein determining the joint UPM is based on the posterior distributions of pᵢ and qᵢ, according to Bayes' rule.

32. The method of any of embodiments 1-31, wherein the toxicity and efficacy probability intervals are associated with a rate of a toxic outcome or a rate of a response outcome, respectively.

33. The method of any of embodiments 1-32, wherein pᵢ is a ratio of a number of subjects experiencing a toxic outcome (xᵢ) to a total number of subjects (nᵢ), and qᵢ is a ratio of the number of subjects experiencing a response outcome (yᵢ) to the total number of subjects (nᵢ).

34. The method of embodiment 32 or embodiment 33, wherein the toxicity outcome is a dose-limiting toxicity (DLT).

35. The method of embodiment 32 or embodiment 33, wherein the response outcome is a complete response (CR).

36. The method of any of embodiments 1-35, wherein the instructions comprise one or more decision tables.

37. The method of any of embodiments 33-36, wherein dose recommendations are provided for one or more possible combinations of and yᵢ among nᵢ subjects.

38. The method of any of embodiments 33-37, wherein dose recommendations are provided for all possible combinations of and yᵢ among nᵢ subjects.

39. The method of any of embodiments 33-38, wherein nᵢ is within a range from about 1 to about 100, about 3 to about 60, or about 6 to about 30.

40. The method of any of embodiments 1-39, wherein the clinical trial is a Phase I clinical trial.

41. The method of any of embodiments 1-40, wherein the clinical trial is a Phase I/II clinical trial.

42. The method of any of embodiments 1-41, wherein the method is a computer implemented method, and wherein one or more of steps of obtaining a matrix, determining a joint unit probability mass (UPM) for each combination of toxicity and efficacy probability intervals, identifying the combination with the highest joint UPM, assigning the dosing action associated with the identified combination, and producing or outputting instructions occur at an electronic device comprising one or more processors and memory.

43. The method of any of embodiments 1-41, wherein the method is a computer implemented method, and wherein one or more of steps a)-e) occur at an electronic device comprising one or more processors and memory.

44. The method of any of embodiments 1-41, wherein the method is a computer implemented method, and wherein one or more steps occur at an electronic device comprising one or more processors and memory.

45. A computer system comprising a processor and memory, the memory comprising instructions operable to cause the processor to carry out one or more of steps of the method of any of embodiments 1-44, wherein the steps are selected from obtaining a matrix, determining a joint unit probability mass (UPM) for each combination of toxicity and efficacy probability intervals, identifying the combination with the highest joint UPM, assigning the dosing action associated with the identified combination, and producing or outputting instructions.

46. A computer system comprising a processor and memory, the memory comprising instructions operable to cause the processor to carry out one or more of steps a)-e) of the methods of any of embodiments 1-44.

47. A computer system comprising a processor and memory, the memory comprising instructions operable to cause the processor to carry out one or more of steps of the method of any of embodiments 1-44.

48. Dose recommendation instructions for performing a clinical trial produced or outputted by the method of any of embodiments 1-47.

49. A method of dosing a subject in a clinical trial for treating a disease or condition, comprising:

a) selecting a dose recommendation for administering a therapeutic agent to a subject that has a disease or condition based on the instructions produced by the methods of any of embodiments 1-44, wherein the dose recommendation is selected for a given combination of a number of subjects experiencing a toxic outcome (xᵢ) and a number of subjects experiencing a response outcome (yᵢ) for a total number of subjects previously treated with the therapeutic agent in the clinical trial at a current dose level i (nᵢ);

b) administering the therapeutic agent to the subject at a dose level in accord with the selected dose recommendation.

50. A method of dosing a subject with a therapeutic agent in a clinical trial for treating a disease or condition, comprising:

a) obtaining instructions that specify dose recommendations, wherein the instructions were produced by:
i) designating two or more toxicity probability intervals of a therapeutic agent and two or more efficacy probability intervals of the therapeutic agent;
ii) assigning a dosing action to each combination of toxicity and efficacy probability intervals;
iii) determining the joint unit probability mass (UPM) for each combination of toxicity and efficacy probability intervals for one or more possible toxicity probabilities at current dose level i (pᵢ) and one or more possible efficacy probabilities at current dose level i (qᵢ);
iv) identifying the combination of toxicity and efficacy intervals that has the highest joint UPM; and
v) assigning the dosing action associated with the identified combination as a dose recommendation for each of the one or more possible toxicity and efficacy probabilities, thereby producing the instructions;

b) selecting a dose recommendation for administering a therapeutic agent to a subject that has a disease or condition based on the instructions for a given combination of a number of subjects experiencing a toxic outcome (xᵢ) and a number of subjects experiencing a response outcome (yᵢ) for a total number of subjects previously treated with the therapeutic agent in the clinical trial at a current dose level i (nᵢ); and

c) administering the therapeutic agent to the subject at a dose level according to the selected dose recommendation.

51. A method of dosing a subject for treating a disease or condition, comprising: a) selecting a dose recommendation for administering a therapeutic agent to a subject that has a disease or condition based on the instructions produced by the methods of any of embodiments 1-44, wherein the dose recommendation is selected for a given combination of a number of subjects experiencing a toxic outcome (xᵢ) and a number of subjects experiencing a response outcome (yᵢ) for a total number of subjects previously treated with the therapeutic agent at a current dose level i (nᵢ);

b) administering the therapeutic agent to the subject at a dose level in accord with the selected dose recommendation.

52. A method of dosing a subject with a therapeutic agent for treating a disease or condition, comprising:

a) obtaining instructions that specify dose recommendations, wherein the instructions were produced by:
i) designating two or more toxicity probability intervals of a therapeutic agent and two or more efficacy probability intervals of the therapeutic agent;
ii) assigning a dosing action to each combination of toxicity and efficacy probability intervals;
iii) determining the joint unit probability mass (UPM) for each combination of toxicity and efficacy probability intervals for one or more possible toxicity probabilities at current dose level i (pᵢ) and one or more possible efficacy probabilities at current dose level i (qᵢ);
iv) identifying the combination of toxicity and efficacy intervals that has the highest joint UPM; and
v) assigning the dosing action associated with the identified combination as a dose recommendation for each of the one or more possible toxicity and efficacy probabilities, thereby producing the instructions;

b) selecting a dose recommendation for administering a therapeutic agent to a subject that has a disease or condition based on the instructions for a given combination of a number of subjects experiencing a toxic outcome (xᵢ) and a number of subjects experiencing a response outcome (yᵢ) for a total number of subjects previously treated with the therapeutic agent in the clinical trial at a current dose level i (nᵢ); and

c) administering the therapeutic agent to the subject at a dose level according to the selected dose recommendation.

53. A method of dosing a subject with a therapeutic agent for treating a disease or condition, comprising:

administering a therapeutic agent to a subject that has a disease or condition based at a dose level according to a selected dose recommendation selected from instructions for a given combination of a number of subjects experiencing a toxic outcome (xᵢ) and a number of subjects experiencing a response outcome (yᵢ) for a total number of subjects previously treated with the therapeutic agent at a current dose level i (nᵢ);

wherein the instructions were produced by:
i) designating two or more toxicity probability intervals of a therapeutic agent and two or more efficacy probability intervals of the therapeutic agent;
ii) assigning a dosing action to each combination of toxicity and efficacy probability intervals;
iii) determining the joint unit probability mass (UPM) for each combination of toxicity and efficacy probability intervals for one or more possible toxicity probabilities at current dose level i (pᵢ) and one or more possible efficacy probabilities at current dose level i (qᵢ);
iv) identifying the combination of toxicity and efficacy intervals that has the highest joint UPM; and
v) assigning the dosing action associated with the identified combination as a dose recommendation for each of the one or more possible toxicity and efficacy probabilities, thereby producing the instructions.

54. The method of any of embodiments 41-42b, wherein therapeutic agent is administered for a clinical trial.

55. The method of any of embodiments 49-54, wherein the dosing action and/or dose recommendation is escalate (E) to dose level i+1.

56. The method of any of embodiments 49-54, wherein the dosing action and/or dose recommendation is stay (S) at dose level i.

57. The method of any of embodiments 49-54, wherein the dosing action and/or dose recommendation is de-escalate (D) to dose level i−1.

58. The method of any of embodiments 50 and 52-57, wherein prior to step v) the dose recommendation is altered to:

1) de-escalate and not return to current dose if the probability that pᵢ is greater than the maximum acceptable toxicity probability (p_{T}) exceeds 0.95;

2) de-escalate and not return to current dose if the probability that qᵢ is less than the minimum acceptable efficacy probability (q_{E}) exceeds 0.7; or

3) escalate and not return to current dose if the probability that qᵢ is less than q_{E} exceeds 0.7.

59. The method of any of embodiments 50 and 52-58, wherein prior to step v) the dose recommendation is altered to:

(1) de-escalate and not return to current dose if pᵢ is greater than p_{T};

(2) de-escalate and not return to current dose if qᵢ is less than q_{E}; or

(3) escalate and not return to current dose if qᵢ is less than q_{E}.

60. The method of embodiment 58 or embodiment 59, wherein the dose recommendation is de-escalate and do not return to the current dose level or any higher dose level (DU).

61. The method of embodiment 58 or embodiment 59, wherein the dose recommendation is de-escalate and do not return to the current dose level or any higher dose level (DEU).

62. The method of embodiment 58 or embodiment 59, wherein the dose recommendation is escalate and do not return to the current dose level or any lower dose level (EEU).

63. The method of any of embodiments 50 and 52-62, wherein prior to step i) the maximum acceptable toxicity probability (p_{T}) and minimum acceptable efficacy probability (q_{E}) of the therapeutic agent are obtained.

64. The method of any of embodiments 50 and 52-63, wherein the joint UPM (JUPM) is determined as:

$$JUPM \begin{matrix}( c , d ) \\ ( a , b )\end{matrix} ≡ \frac{Pr ( {p}_{i} ∈ ( a , b ) , {q}_{i} ∈ ( c , d ) \vert 𝒟 )}{( b - a ) \times ( d - c )} , \\ 0 < a < b < 1 , 0 < c < d < 1. \qquad ( 1 )$$

65. The method of any of embodiments 50 and 52-64, wherein pᵢ is a ratio of a number of subjects experiencing a toxic outcome (xᵢ) to a total number of subjects (nᵢ), and qᵢ is a ratio of the number of subjects experiencing a response outcome (yᵢ) to the total number of subjects (nᵢ).

66. The method of any of embodiments 49-65, wherein the toxicity outcome is a dose-limiting toxicity (DLT).

67. The method of any of embodiments 49-66, wherein the response outcome is a complete response (CR).

68. The method of any of embodiments 49-67, wherein the selected dose is determined based on the number of subjects previously treated in the clinical trial and the actual probabilities of the toxic outcomes and response outcomes among the subjects previously treated.

69. The method of any of embodiments 49-68, wherein the subject is part of a cohort of subjects and all subjects in the cohort are administered the therapeutic agent at the same dose level.

70. The method of any of embodiments 49-69, that is repeated for remaining subjects in the clinical trial.

71. The method of any of embodiments 49-70, wherein the clinical trial in terminated when the number of subjects enrolled reaches a pre-specified maximum.

72. The method of embodiment 71, wherein the pre-specified maximum is within a range from about 1 to about 100, about 3 to about 60, or about 6 to about 30.

73. The method of any of embodiments 49-72, further comprising identifying the optimal dose level, wherein the optimal dose level is associated with the highest probability that pᵢ is less than p_{T} and qᵢ is less than q_{E}.

74. The method of any of embodiments 49-73, wherein the optimal dose level is identified based on a combined utility function determined from safety and efficacy utility functions.

75. The method of embodiment 74, wherein the combined utility function is determined as:

U(p,q)=ƒ₁(p)ƒ₂(q)

76. The method of any of embodiments 49-75, wherein the optimal dose level comprises the largest combined posterior utility.

77. The method of embodiment 76, wherein the combined posterior utility is determined as:

î=argmaxᵢE[U(pᵢ,qᵢ)| [symbol] ]

78. The method of any of embodiments 49-77, wherein the clinical trial is a Phase I clinical trial.

79. The method of any of embodiments 49-78, wherein the clinical trial is a Phase I/II clinical trial.

80. The method of any of embodiments 49-79, wherein the disease or condition is a tumor or a cancer.

81. The method of any of embodiments 1-80, wherein the therapeutic agent is for treating a tumor or a cancer.

82. The method of any of embodiments 1-81, wherein the therapeutic agent is one for which the response outcome can be assessed within the timeframe in which the toxicity outcome is assessed.

83. The method of any of embodiments 1-82, wherein the therapeutic agent is or a small molecule, a gene therapy, a transplant or an adoptive cell therapy.

84. The method of any of embodiments 1-83, wherein the therapeutic agent is or comprises an adoptive cell therapy.

85. The method of embodiment 84, wherein the adoptive cell therapy comprises cells expressing a chimeric antigen receptor (CAR).

86. The method of embodiments 85, wherein the CAR expressed by the cells specifically binds to an antigen expressed by a cell or tissue of the disease or condition.

87. The method of any of embodiments 84-86, wherein the cells are administered at a dose level that is between about 0.5×10⁶ cells/kg body weight of the subject and 6×10⁶ cells/kg, between about 0.75×10⁶ cells/kg and 2.5×10⁶ cells/kg, between about 1×10⁶ cells/kg and 2×10⁶ cells/kg, between about 2×10⁶ cells per kilogram (cells/kg) body weight and about 6×10⁶ cells/kg, between about 2.5×10⁶ cells/kg and about 5.0×10⁶ cells/kg, or between about 3.0×10⁶ cells/kg and about 4.0×10⁶ cells/kg, each inclusive.

88. The method of any of embodiments 84-87, wherein the number of cells administered is between about 1×10⁶ and about 1×10⁸ CAR expressing cells, between about 2×10⁶ and about 5×10⁶ CAR expressing cells, between about 1×10⁷ and about 5×10⁷ CAR expressing cells, or between about 5×10⁷ and about 1×10⁸ total CAR expressing cells.

89. The method of any of embodiments 84-88, wherein the dose of cells are administered in a single pharmaceutical composition comprising the cells of the dose.

90. The method of any of embodiments 84-89, wherein:

the dose is a split dose, wherein the cells of the dose are administered in a plurality of compositions, collectively comprising the cells of the dose, which, optionally, are administered over a period of no more than three days.

91. The method of any of embodiments 49-90, wherein the disease or condition is a leukemia or lymphoma.

92. The method of any of embodiments 49-91, wherein the disease or condition is acute lymphoblastic leukemia.

93. The method of any of embodiments 49-91, wherein the disease or condition is a non-Hodgkin lymphoma (NHL).

### IX. Examples

The following examples are included for illustrative purposes only and are not intended to limit the scope of the invention.

### Example 1: Dose Recommendation Based on Toxicity and Efficacy Probability Intervals (TEPI)

A toxicity and efficacy probability interval (TEPI) method was designed in which both safety and efficacy data are used to inform dose escalation decisions. The TEPI model can be used for determining dose recommendations in a clinical trial, e.g., Phase I trial. Based on this design, an optimal dose level can be selected by jointly considering safety, e.g. dose-limiting toxicity (DLT), and efficacy, e.g., response rate. The approach provides adaptive features and is easy and transparent for a clinician to implement. The method can be adapted for use in the design and implementation of clinical trials, e.g. Phase I dose-finding trials, for testing any therapy for which toxicity and efficacy response can be assessed in the same timeframe. Using the model, once a clinical trial is complete, the dose level with the largest combined safety and efficacy utility can be chosen as the optimal dose level.

The TEPI model is based on pre-defined dosing actions as outlined below, which are then used to generate instructions for dose recommendations that can be displayed in a Decision Table for use in making dosing decisions about a therapeutic agent, which, in some aspects, can be carried out by a clinician or non-statistician during a clinical trial.

A. Design of Matrix (e.g. Preset Table)

In the method, a preset matrix (e.g. Table) is generated based on combinations of toxicity and efficacy probability intervals that consider information about both toxicity and efficacy for a particular therapeutic agent (e.g. CAR T cell therapy), including the maximum acceptable toxicity probability, p_{T}, and the minimum acceptable efficacy probability. The maximum acceptable toxicity probability, p_{T}, and the minimum acceptable efficacy probability, e.g., antitumor activity, q_{E}, can be defined with input from the physician, clinician or other non-statistician and may vary based on the particular therapeutic agent, patient population or disease or condition to be treated. A two-way matrix based on a combination of toxicity and efficacy probability intervals each assigned a toxicity grade (e.g., low, moderate, high, unacceptable) and efficacy grade (e.g., low, moderate, high, superb) can be prepared with input from a physician, clinician or other non-statistician in which each combination of toxicity and efficacy grade is associated with a dosing action, such as “Escalate” (E), “Stay” (S), “De-escalate” (D), Unacceptable efficacy (EU) and Unacceptable toxicity (denoted interchangeably as DU or DU_{T}).

Information about both toxicity and efficacy of the therapy is considered. For example, for d ascending dose levels in a single-agent therapy Phase I trial, pᵢ and qᵢ denote the probability of toxicity and efficacy for the i^{th} dose, respectively. It is assumed that the toxicity probability pᵢ increases with dose level i (i.e., p₁≤p₂≤p_{d}). On the other hand, the efficacy probability qᵢ may first increase initially and then reach a plateau or increase with only minimum improvement. For this reason, pᵢ and qᵢ are assumed to be independent. In one example, dose i is currently used in the trial and m subjects have already been allocated to dose i, with xᵢ and yᵢ subjects experiencing toxicity and efficacy outcomes, respectively. Thus, xᵢ and yᵢ are assumed to be independently distributed. The trial data is denoted as: D={(nᵢ, xᵢ, yᵢ), i=1, . . . , d}.

The matrix of dosing actions assigned to combinations of toxicity and efficacy intervals can be displayed in a matrix (e.g. table) that acts as a “preset table” for the design. Each combination, e.g. rectangle, corresponds to a combination of toxicity and probability interval and is assigned a specific dosing action. In general, the practical dosing action can be assigned for each therapeutic agent, typically by a clinician or physician or other person with knowledge of the potential toxicity and/or efficacy of the therapeutic agent. In some cases, the dosing action decisions that are assigned reflect practical clinical actions that would likely result if the particular combination of toxicity and efficacy data is observed at a certain dose level. Dosing action “E” denotes escalation, i.e., treating subjects at the next higher dose (i+1). Dosing action “S” denotes staying at the current dose level i for future subjects. Dosing action “D” denotes de-escalation, i.e., treating subjects at the next lower dose level (i−1).

Table 4A and Table 4B are each exemplary preset tables in which there are 16 combinations associated with efficacy and toxicity (e.g. each set forth in a two-dimensional rectangle of a Table). In Table 4A, dosing action “DU” encompasses “D”, and “U”, which means that the current dose level is unacceptable due to high toxicity and will be excluded in the trial for the following cohorts and dosing action “EU” encompasses “E” and “U,” which means that the current dose level has unacceptable efficacy and the decision is to escalate and never return due to unacceptable low efficacy

In the preset tables, each combination of toxicity and efficacy grade is associated with a dosing action. The physician-specified dosing actions for a range of {(a, b), (c, d)} values anchors the statistical inference in the dose-finding protocol. For example, a low toxicity and low efficacy corresponds to a two-dimensional rectangle for pᵢ and qᵢ, respectively: (0, 0.15)×(0, 0.2) as shown in Table 4A. As shown in Table 4A, this combination of toxicity and efficacy may be associated with E, escalation, and/or EU, unacceptable efficacy.

TABLE 4A

| | | | Efficacy | | | |
|---|---|---|---|---|---|---|
| | | | | Mod- | | |
| | | | Low | erate | High | Superb |
| | | | (0.0- | (0.2- | (0.4- | (0.6- |
| | | | 0.2) | 0.4) | 0.6) | 1) |
| Toxicity | Low | (0.0-0.15) | E or | E | E | E |
| | | | EU | | | |
| | Moderate | (0.15-0.33) | E or | E | E | S |
| | | | EU | | | |
| | High | (0.33-0.4) | D | S | S | S |
| | Unaccept- | (0.4-1) | D or | D or | D or | D or |
| | able | | DU | DU | DU | DU |

EU = unacceptable efficacy;
E = escalate;
S = stay;
D = de-escalate;
DU = unacceptable toxicity

TABLE 4B

| | | | Efficacy | | | |
|---|---|---|---|---|---|---|
| | | | Unaccept- | Mod- | | |
| | | | able | erate | High | Superb |
| | | | (0.0- | (0.2- | (0.4- | (0.6- |
| | | | 0.2) | 0.4) | 0.6) | 1) |
| Toxicity | Low | (0.0-0.15) | E | E | E | E |
| | Moderate | (0.15-0.33) | E | E | E | S |
| | High | (0.33-0.4) | D | S | S | S |
| | Unaccept- | (0.4-1) | D | D | D | D |
| | able | | | | | |

E = escalate;
S = stay;
D = de-escalate;

B. Determining Joint Unit Probability Mass (UPM)

The design a priori assumes that both DLT (dose-limiting toxicity) and response rate, e.g., efficacy, follow a uniform beta(1,1) distribution. The design uses a beta-binomial model to compute the posterior probabilities of the toxicity and efficacy intervals based on safety and efficacy data, which can be determined from a current cohort or other information available to the clinician or physician.

The design utilizes information on the computed joint unit probability mass (UPM) of toxicity and efficacy data, which follows the Bayes rule under independent beta prior distributions. For a given region A, the joint UPM is defined as the ratio between the probability of the region and the size of the region. Considering the two-dimensional unit square (0, 1)×(0, 1) in the real space, the joint UPM (JUPM) for the rectangular region of (a, b)×(c, d) is:

$$JUPM \begin{matrix}( c , d ) \\ ( a , b )\end{matrix} ≡ \frac{Pr ( {p}_{i} ∈ ( a , b ) , {q}_{i} ∈ ( c , d ) \vert 𝒟 )}{( b - a ) \times ( d - c )} , \\ 0 < a < b < 1 , 0 < c < d < 1. \qquad ( 1 )$$

Here, the numerator is the posterior probability of pᵢ and qᵢ in the interval (a, b) and (c, d), respectively.

It is assumed that pᵢ and qᵢ have a beta prior distribution Beta(x; α, β), so the posterior distributions for pᵢ and qᵢ are independent and follow Beta(x; α+xᵢ, β+nᵢ−xᵢ) and Beta(x; α+yᵢ, β+nᵢ−yᵢ), respectively. Based on the posterior distributions, there is a winning combination (a*, b*)×(c*, d*) that achieves the maximum UPM among all the combinations in a preset table (e.g., see Table 4A or 4B), and corresponding dosing actions will be provided as dose recommendations for dosing the next cohort of subjects. Such an approach follows the Bayes' Rule under 0/1 loss function. The dose recommendations can be determined prior to the start of a trial considering any of a number of possible permutations of toxicity and efficacy outcomes.

C. Safety and Futility Rules

Safety and futility also are considered in the model by two additional rules that are added to the dose finding protocol. One is to exclude the dose with excessive toxicity (safety rule) and the other one is to exclude the dose with very low efficacy (futility rule)

The safety rule is implemented as follows:

if Pr(pᵢ>p_{T}(data)>η for η close to 1 (e.g. 0.95), exclude dose i, i+1 . . . d for further use in this trial (i.e. these doses will never be tested again in the trail), and treat the next cohort of patients at dose i−1. This corresponds to a dosing action of “DU” (denoted interchangeably as “DU_{T}”, de-escalate due to unacceptable high toxicity).

The futility rule is implemented as follows:

if Pr(qᵢ<q_{E}|data)>ξ for a small ζ (e.g. 0.3), exclude dose i, i−1, . . . ,d for further use in the trial. This corresponds to a dosing action of “EEU” (denoted interchangeably as EU; escalate and never return due to unacceptable low efficacy) or “DEU” (denoted interchangeably as DU_{E}; deescalate and never return to this dose due to unacceptable low efficacy).

For the above rules, p_{T} is the highest toxicity rate that can be tolerated and q_{E} is the lowest efficacy rate that is deemed acceptable.

A dose satisfying both the safety rule and the futility rule is considered an “available” dose. Only available dose levels can be used to treat subjects in the trial.

D. Decision Table

Combining the safety and futility rules and the Bayes' Rule that maximizes UPMs, a trial design protocol is prepared for a particular desired therapeutic agent dosing design, such as in connection with a clinical trial, as follows:
If no dose is available, the trial is terminated.
The current dose is the dose used to treat the current cohort of subjects.
If the current dose violates the safety rule, de-escalate to the maximum available dose below the current dose.
If the current dose violates the futility rule,
   If the decision is “E”, escalate to the closest available dose above the current dose. If no dose levels above the current dose level are available, de-escalate the dose to the closest available dose below the current dose. This rule is justified because efficacy may not always be monotone. Therefore, when the current dose is not effective, an effective dose could be either a higher dose or a lower dose.
   If the decision is “D”, de-escalate to the closest available dose below the current dose. In no dose levels below the current dose are available, terminate the trial.
   If the decision is “DU”, mark the current dose and all the higher dose levels as unavailable. If no dose levels are available, terminate the trial. If there are still available dose levels below the current dose, de-escalate the dose to the closest available dose below the current dose.
   If the decision is “S”, de-escalate the dose to the closest available dose below the current dose. If no dose below the current dose is available, terminate the trial.
If the current dose satisfies both the safety rule and the futility rule,
   If the decision is “E”, escalate the dose to the closest available dose above the current dose. If no dose levels above the current dose are available, stay at the current dose.
   If the decision is “D”, de-escalate to the closest available dose below the current dose. If no dose levels below the current dose are available, stay at the current dose.
   If the decision is “DU”, mark the current dose level and all higher dose levels as unavailable. If no dose levels are available, terminate the trial. If there are still available dose levels below the current dose level, de-escalate the dose to the closest available dose level below the current dose level.
   If the decision is “S”, stay at the current dose level.

The dose recommendation instructions can be compiled into a dose recommendation Decision Table. An exemplary dose recommendation Decision Table based on the preset decisions in Table 4A are shown in Tables 5A and 5B for various numbers of subjects (n) treated at each dose level. An exemplary dose recommendation Decision Table based on the preset decisions in Table 4B are shown in Tables 6A and 6B for various numbers of subjects (n) treated at each dose level.

In summary, the dose-finding method of the TEPI model is as follows. The TEPI model assumes that a current patient cohort is treated at dose i. After the current cohort of patients completes DLT and response evaluation, the JUPM's for all the interval combinations in a preset matrix (e.g. preset Table), such as is shown in exemplary Tables 4A and 4B, can be determined to result in dose recommendation instructions, such as shown in the exemplary Tables 5A and 5B or Tables 6A and 6B. As described, the TEPI model design recommends that “E”, “S” or “D” dosing action decisions correspond to the combination with the largest JUPM value. Therefore, based on a preset matrix (e.g. Table 4A or 4B), all the decisions can be precalculated and presented as dose recommendation instructions or Decision Table (e.g. Table 5A or 5B or Tables 6A and 6B).

TABLE 5A

| No. of Treated | | | | |
|---|---|---|---|---|
| Subjects at | | | | |
| Current Dose Level | No. of DLTs | No. of Responders | | |
| | | 0 | 1-3 | |
| 3 | 0 | E | E | |
| | 1 | D | S | |
| | 2-3 | D or DU | D or DU | |
| | | 0 | 1-4 | 5-6 |
| 6 | 0 | EEU | E | E |
| | 1 | EEU | E | S |
| | 2-3 | DEU | D | S |
| | 4 | DEU | D | D |
| | 5-6 | DU | DU | DU |
| | | 0-1 | 2-6 | 7-9 |
| 9 | 0-1 | EEU or E | E | E |
| | 2 | EEU or E | E | S |
| | 3-4 | DEU or D | S | S |
| | 5-9 | DEU or DU | D or DU | D or DU |

E: escalate;
S: stay;
D: de-escalate;
EU: Pr (Response rate < 0.2|data) > 0.70;
DU: Pr (DLT > 0.4|data) > 0.95;
EEU: Escalate and not return to this dose level due to futility;
DEU: De-escalate and not return to this dose level due to futility

TABLE 5B

| No. of Treated | | | | |
|---|---|---|---|---|
| Subjects at Current | | | | |
| Dose Level | No. of DLTs | No. of Responders | | |
| | | 0-2 | 3-7 | 8-12 |
| 12 | 0-1 | EEU or E | E | E |
| | 2-3 | EEU or E | E | S |
| | 4-6 | DEU or D | S | S |
| | 7 | DEU | D | D |
| | 8-12 | DU | DU | DU |
| | | 0-2 | 3-9 | 10-15 |
| 15 | 0-2 | EEU or E | E | E |
| | 3-4 | EEU or E | E | S |
| | 5-7 | DEU or D | S | S |
| | 8-9 | DEU or D | D | D |
| | 10-15 | D or DU | D or DU | D or DU |

E: escalate;
S: stay;
D: de-escalate;
EU: Pr (Response rate < 0.2|data) > 0.70;
DU: Pr (DLT > 0.4|data) > 0.95;
EEU: Escalate and not return to this dose level due to futility;
DEU: De-escalate and not return to this dose level due to futility

TABLE 6A

| Number of Patients Treated | | | | | |
|---|---|---|---|---|---|
| at Current Dose Level | No. of DLTs | No. of Responders | | | |
| | | 0 | 1-3 | | |
| 3 | 0 | E | E | | |
| | 1 | D | S | | |
| | 2 | D | D | | |
| | 3 | DU_{T} | DU_{T} | | |
| | | 0 | 1-4 | 5-6 | |
| 6 | 0 | EU | E | E | |
| | 1 | EU | E | S | |
| | 2-3 | DU_{E} | D | S | |
| | 4 | DU_{E} | D | D | |
| | 5-6 | DU_{T} | DU_{T} | DU_{T} | |
| | | 0 | 1 | 2-6 | 7-9 |
| 9 | 0-1 | EU | E | E | E |
| | 2 | EU | E | E | S |
| | 3-4 | DU_{E} | D | S | S |
| | 5-6 | DU_{E} | D | D | D |
| | 7-9 | DU_{T} | DU_{T} | DU_{T} | DU_{T} |
| | | 0-1 | 2 | 3-7 | 8-12 |
| 12 | 0-1 | EU | E | E | E |
| | 2 | EU | E | E | S |
| | 3-5 | DU_{E} | D | S | S |
| | 6 | DU_{E} | D | D | D |
| | 7-12 | DU_{T} | DU_{T} | DU_{T} | DU_{T} |
| | | 0-1 | 2 | 3-9 | 10-15 |
| 15 | 0-2 | EU | E | E | E |
| | 3-4 | EU | E | E | S |
| | 5-7 | DU_{E} | D | S | S |
| | 8-9 | DU_{E} | D | D | D |
| | 10-15 | DU_{T} | DU_{T} | DU_{T} | DU_{T} |

DU_{T}: De-escalate due to toxicity, current dose unacceptable for future use
DU_{E}: De-escalate due to low efficacy, current dose unacceptable for future use
EU: Escalate and current dose, unacceptable for future use

TABLE 6B

| Number of Patients Treated | | | | | |
|---|---|---|---|---|---|
| at Current Dose Level | No. of DLTs | No. of Responders | | | |
| | | 0-2 | 3 | 4-11 | 12-18 |
| 18 | 0-2 | EU | E | E | E |
| | 3-5 | EU | E | E | S |
| | 6-9 | DU_{E} | D | S | S |
| | 10 | DU_{E} | D | D | D |
| | 11-18 | DU_{T} | DU_{T} | DU_{T} | DU_{T} |
| | | 0-2 | 3 | 4-13 | 14-21 |
| 21 | 0-2 | EU | E | E | E |
| | 3-6 | EU | E | E | S |
| | 7-10 | DU_{E} | D | S | S |
| | 11-12 | DU_{E} | D | D | D |
| | 13-21 | DU_{T} | DU_{T} | DU_{T} | DU_{T} |
| | | 0-3 | 4 | 5-15 | 16-24 |
| 24 | 0-3 | EU | E | E | E |
| | 4-6 | EU | E | E | S |
| | 7-12 | DU_{E} | D | S | S |
| | 13 | DU_{E} | D | D | D |
| | 16-24 | DU_{T} | DU_{T} | DU_{T} | DU_{T} |
| | | 0-3 | 4-5 | 6-17 | 18-27 |
| 27 | 0-3 | EU | E | E | E |
| | 4-7 | EU | E | E | S |
| | 8-13 | DU_{E} | D | S | S |
| | 14 | DU_{E} | D | D | D |
| | 16-24 | DU_{T} | DU_{T} | DU_{T} | DU_{T} |

DU_{T}: De-escalate due to toxicity, current dose unacceptable for future use
DU_{E}: De-escalate due to low efficacy, current dose unacceptable for future use
EU: Escalate and current dose, unacceptable for future use

E. Determination of Optimal Dose Level

At the end of a trial, a utility score, which balances the toxicity and efficacy information, of each dose can be calculated and the highest expected utility score can be determined. The optimal dose level can then be chosen based on the joint utility of safety and efficacy. An elicited utility function for safety and efficacy can be constructed based on maximally tolerable safety and minimally acceptable efficacy parameters, which, in some cases, can be constructed with input from a clinician or physician.

When the number of subjects enrolled in a particular trial reaches a pre-specified maximum sample size, the trial is terminated and the optimal dose level is selected. The dose level with the highest probability Pr(pᵢ<p_{T}, qᵢ>q_{E}+δ|data) is selected as the optimal dose level. Here, δ is the expected increment over the minimum efficacy rate q_{E} for the therapy.

FIG. 1 shows exemplary individual utility graphs for safety and efficacy. FIG. 1 (top) shows an exemplary utility function f₁(p) for safety, in which utility is defined as 1 if the DLT rate is less than or equal to 20% and conversely, utility is defined as 0 if the DLT rate is greater than 40%. As shown in FIG. 1, between a 20% and 40% DLT rate, utility decreases linearly as the DLT rate increases. FIG. 1 (bottom) shows an exemplary f₂(q) for efficacy, which is defined as 0 if the response rate is less than 20%, beyond a 60% response rate, the utility is defined as 1 and between 20% and 60% utility increases linearly as the response rate increases.

The utility score assesses all available doses by incorporating both their toxicity and efficacy rates, which, in some cases, are conducted prior to a particular trial. Apart from the independent prior assumption for pᵢ's in the dose finding step, a monotonic constraint on priors for pᵢ's is assumed while selecting the best dose, i.e. p1<p₂> . . . pd. The optimal dose level is selected based on a utility function determined from both safety and efficacy utility functions:

u(p,q)=ƒ₁(p)ƒ₂(q)

where p denotes the toxicity rate and q denotes the efficacy rate and where f₁(p) is decreasing with p, and f₂(q) is increasing with q. Both f1(⋅) and f2(⋅) are truncated linear functions given by:

$${f}_{1} ( p ) = \{ \begin{matrix}1 , & p ∈ ( 0 , {p}_{i}^{*} ] , \\ 1 - \frac{p - {p}_{1}^{*}}{{p}_{2}^{*} - {p}_{1}^{*}} , & p ∈ ( {p}_{1}^{*} , {p}_{2}^{*} ) , \\ 0 , & p ∈ [ {p}_{2}^{*} , 1 ) ,\end{matrix} and \\ {f}_{2} ( q ) = \{ \begin{matrix}0 , & q ∈ ( 0 , {q}_{i}^{*} ] , \\ \frac{q - {q}_{1}^{*}}{{q}_{2}^{*} - {q}_{1}^{*}} , & q ∈ ( {q}_{1}^{*} , {q}_{2}^{*} ) , \\ 1 , & q ∈ [ {q}_{2}^{*} , 1 ) ,\end{matrix}$$

The posterior expected utility, E[U(pᵢ, qᵢ)|D], can be computed using a numerical approximation for each dose i. In this approximation, a total of T random samples are generated from the posterior distributions. For each sample t, p^{t}=(p^{t}₁, . . . , p^{t}_{d}) and q^{t}=(q^{t}₁, . . . , q^{t}_{d}) are generated. An isotonic transformation is performed (Ji et al., 2007; 2010) on p^{t} to obtain \hat{ }p^{t}=(p\hat{ }^{t}₁, . . . , p\hat{ }^{t}_{d}) where p\hat{ }ᵢ^{t}≤p\hat{ }^{t}ⱼ if i<j, which may ensure that p^{t} is non-decreasing. Based on the samples qᵢ^{t} and p\hat{ }ᵢ^{t}, a corresponding utility score U^{t}(p\hat{ }ᵢ^{t}, qᵢ^{t}) can be calculated according to above for each dose i. Then, the estimated posterior expected utility is given by:

$$\hat{E} [ U ( {p}_{i} , {q}_{i} ) \vert D ] = \frac{1}{T} {\sum }_{t = 1}^{T} {U}^{t} ( {\hat{p}}_{i}^{t} , {q}_{i}^{t} )$$

The dose level with the largest expected posterior utility, such as according to the following formula, is selected:

î=argmaxᵢE[U(pᵢ,qi)| [symbol] ]

### Example 2: Clinician Dosing According to TEPI Model

The clinician chooses the starting dose, the maximum acceptable toxicity, and the minimum acceptable efficacy, e.g., antitumor activity. The preset table is derived as described above. The clinician reviews the preset table to ensure it reflects clinical practice during a trial. The intervals can be calibrated as needed.

Once the dose recommendation Decision Table is generated, each cohort is treated at the appropriate dose level. Untried dose levels are not skipped. If no dose level is acceptable based on combined futility and safety monitoring, the trial is stopped. Otherwise, if no early stopping criteria are met, the trial is stopped once it reaches the maximum sample size. At the end of the trial, the optimal dose level based on utility (combined safety and efficacy) is selected.

### Example 3: Clinical Trial Dosage Recommendation Simulations Based on Toxicity and Efficacy Probability Interval (TEPI) Model

The TEPI design described in Example 1 was tested in various simulation scenarios and was compared to other approaches including the modified toxicity probability interval (mTPI) design, the 3+3 design, and the continual reassessment method (CRM). For the simulations, the maximum sample size was 27 subjects and the cohort size was 3 subjects. The simulations assumed dichotomous efficacy and safety outcomes that were independent, with a monotonic relationship between toxicity and dose.

Six scenarios were simulated as follows:
Scenario 1 (Table 7): All doses safe; No dose is efficacious
Scenario 2 (Table 8): Monotone dose-response and one dose with acceptable toxicity and efficacy
Scenario 3 (Table 9): Dose safe with increased toxicity and descending efficacy
Scenario 4 (Table 10): Two higher doses unsafe and all doses efficacious but dose level 2 and 3 with similar efficacy
Scenario 5 (Table 11): All doses safe with similar toxicity but different efficacy
Scenario 6 (Table 12): All doses efficacious but no dose safe

For each scenario, the study was designed to include up to four potential dose levels. Within each of the scenarios, the true toxicity probability and efficacy probability are listed in each of Tables 7-12, respectively. In the simulation, the following parameters were set for the TEPI design: the maximum acceptable toxicity rate, p_{T}, was set at 0.3; the minimum acceptable response rate (efficacy), q_{E}, was set at 0.2.

Under each scenario, 1000 trial simulations were conducted in silico. The results are shown in Tables 7-12.

TABLE 7
Scenario #1: All Dose Safe; No Dose is Efficacious

| | True | | | | | | | | | |
|---|---|---|---|---|---|---|---|---|---|---|
| Dose | Prob | True Prob | Selection Prob (%) | | | | # of Subjects Treated | | | |
| Level | (Tox) | (Response) | TEPI | mTPI | 3 + 3 | CRM | TEPI | mTPI | 3 + 3 | CRM |
| 1 | 0.16 | 0.05 | 22.1 | 12.1 | 23.8 | 7.9 | 6.015 | 7.749 | 4.581 | 7.929 |
| 2 | 0.2 | 0.1 | 17.9 | 24.1 | 22 | 23.6 | 5.715 | 8.052 | 3.849 | 7.524 |
| 3 | 0.25 | 0.15 | 17.2 | 30.6 | 16 | 31.9 | 5.121 | 6.12 | 2.742 | 6.306 |
| 4 | 0.3 | 0.18 | 7.5 | 32.1 | 15.8 | 36.6 | 4.326 | 4.854 | 1.437 | 5.241 |

| | TEPI | mTPI | 3 + 3 | CRM |
|---|---|---|---|---|
| Prob of Early | 35.3 | 1.1 | 22.4 | 0 |
| Termination | | | | |
| Average # of | 21.177 | 26.775 | 12.609 | 27 |
| subjects treated | | | | |
| in a trial | | | | |

TABLE 8
Scenario #2: Monotone Dose-Response and
One Dose with Acceptable Toxicity and Efficacy

| | True | | | | | | | | | |
|---|---|---|---|---|---|---|---|---|---|---|
| Dose | Prob | True Prob | Selection Prob (%) | | | | # of Subjects Treated | | | |
| Level | (Tox) | (Response) | TEPI | mTPI | 3 + 3 | CRM | TEPI | mTPI | 3 + 3 | CRM |
| 1 | 0.1 | 0.05 | 15.4 | 5 | 27.3 | 2.1 | 5.124 | 5.709 | 4.443 | 5.688 |
| 2 | 0.2 | 0.2 | 26.7 | 37.1 | 42.5 | 34.3 | 6.84 | 10.089 | 4.797 | 9.297 |
| 3 | 0.35 | 0.6 | 38.5 | 47.8 | 17 | 52.8 | 8.925 | 8.511 | 3.324 | 9.237 |
| 4 | 0.5 | 0.65 | 0.60 | 10.1 | 3.7 | 10.8 | 2.988 | 2.691 | 0.993 | 2.778 |

| | TEPI | mTPI | 3 + 3 | CRM |
|---|---|---|---|---|
| Prob of Early | 18.8 | 0 | 9.5 | 0 |
| Termination | | | | |
| Avg. # of | 23.877 | 27 | 13.557 | 27 |
| subjects treated | | | | |
| in a trial | | | | |

TABLE 9
Scenario #3: All Dose Safe with Increase Toxicity and Descending Efficacy

| | True | | | | | | | | | |
|---|---|---|---|---|---|---|---|---|---|---|
| Dose | Prob | True Prob | Selection Prob (%) | | | | # of Subjects Treated | | | |
| Level | (Tox) | (Response) | TEPI | mTPI | 3 + 3 | CRM | TEPI | mTPI | 3 + 3 | CRM |
| 1 | 0.05 | 0.1 | 9.1 | 1.1 | 19.1 | 0.2 | 3.981 | 3.966 | 3.96 | 3.966 |
| 2 | 0.15 | 0.4 | 41 | 18.7 | 32.2 | 7.9 | 6.285 | 7.878 | 4.62 | 6.207 |
| 3 | 0.25 | 0.7 | 46.8 | 37.6 | 23.8 | 36.2 | 10.293 | 8.367 | 3.954 | 8.397 |
| 4 | 0.3 | 0.3 | 0.60 | 42.6 | 22.1 | 55.7 | 5.991 | 6.789 | 2.124 | 8.43 |

| | TEPI | mTPI | 3 + 3 | CRM |
|---|---|---|---|---|
| Prob of Early | 2.5 | 0 | 2.8 | 0 |
| Termination | | | | |
| Avg. # of | 26.55 | 27 | 14.658 | 27 |
| subjects treated | | | | |
| in a trial | | | | |

TABLE 10
Scenario #4: Two Higher Doses Unsafe and All Doses
Efficacious But Dose Level 2 and 3 With Similar Efficacy

| | True | | | | | | | | | |
|---|---|---|---|---|---|---|---|---|---|---|
| Dose | Prob | True Prob | Selection Prob (%) | | | | # of Subjects Treated | | | |
| Level | (Tox) | (Response) | TEPI | mTPI | 3 + 3 | CRM | TEPI | mTPI | 3 + 3 | CRM |
| 1 | 0.15 | 0.43 | 53.9 | 11.3 | 23.8 | 7.7 | 6.102 | 7.308 | 4.554 | 7.656 |
| 2 | 0.2 | 052 | 41.3 | 41.6 | 41 | 43.8 | 9.567 | 9.585 | 4.314 | 9.723 |
| 3 | 0.4 | 0.5 | 3.6 | 38.8 | 11.7 | 41.7 | 8.954 | 7.926 | 2.829 | 7.593 |
| 4 | 0.5 | 0.6 | 1.20 | 7.1 | 2.5 | 6.8 | 2.136 | 1.938 | 0.681 | 2.028 |

| | TEPI | mTPI | 3 + 3 | CRM |
|---|---|---|---|---|
| Prob of Early | 1.2 | 1.2 | 21 | 0 |
| Termination | | | | |
| Avg. # of | 26.769 | 26.757 | 12.378 | 27 |
| subjects treated | | | | |
| in a trial | | | | |

TABLE 11
Scenario #5: All Doses Safe with Similar Toxicity But Different Efficacy

| | True | | | | | | | | | |
|---|---|---|---|---|---|---|---|---|---|---|
| Dose | Prob | True Prob | Selection Prob (%) | | | | # of Subjects Treated | | | |
| Level | (Tox) | (Response) | TEPI | mTPI | 3 + 3 | CRM | TEPI | mTPI | 3 + 3 | CRM |
| 1 | 0.25 | 0.3 | 46.8 | 35.6 | 25.5 | 36.6 | 9.939 | 12.9 | 4.845 | 13.212 |
| 2 | 0.28 | 0.45 | 31.7 | 26.5 | 15.6 | 25.1 | 7.83 | 25.1 | 2.928 | 7.143 |
| 3 | 0.3 | 0.55 | 9.8 | 18 | 7.7 | 21.6 | 4.38 | 21.6 | 1.617 | 4.086 |
| 4 | 0.33 | 0.7 | 2.90 | 13.2 | 6.4 | 16.7 | 3.036 | 16.7 | 0.66 | 2.559 |

| | TEPI | mTPI | 3 + 3 | CRM |
|---|---|---|---|---|
| Prob of Early | 8.8 | 6.7 | 44.8 | 0 |
| Termination | | | | |
| Avg. # of | 25.185 | 25.794 | 10.05 | 27 |
| subjects treated | | | | |
| in a trial | | | | |

TABLE 12
Scenario #6: All Doses Efficacious But No Dose Safe

| | | | | |
|---|---|---|---|---|
| | True | | | |
| Dose | Prob | True Prob | Selection Prob (%) | # of Subjects Treated |

| Level | (Tox) | (Response) | TEPI | mTPI | 3 + 3 | CRM | TEPI | mTPI | 3 + 3 | CRM |
|---|---|---|---|---|---|---|---|---|---|---|
| 1 | 0.5 | 0.4 | 33.9 | 23 | 10.7 | 99.3 | 14.859 | 13.98 | 4.509 | 25.809 |
| 2 | 0.6 | 0.5 | 0.3 | 0.9 | 0.3 | 0.7 | 1.809 | 1.179 | 0.627 | 1.083 |
| 3 | 0.7 | 0.6 | 0 | 0.1 | 0.2 | 0 | 0.111 | 0.087 | 0.033 | 0.108 |
| 4 | 0.8 | 0.8 | 0.00 | 0 | 0 | 0 | 0 | 0 | 0.009 | 0 |

| | TEPI | mTPI | 3 + 3 | CRM |
|---|---|---|---|---|
| Prob of Early | 65.8 | 76 | 88.8 | 0 |
| Termination | | | | |
| Avg. # of | 16.779 | 15.264 | 5.178 | 27 |
| subjects treated | | | | |
| in trial | | | | |

Under all scenarios, the 3+3 design resulted in the smallest average number of subjects treated, but poorer performance characteristics. TEPI was demonstrated to be safer (treating fewer subjects at toxic doses) and more reliable in finding the optimal dose level than the 3+3 design.

TEPI was shown to be more reliable than mTPI when there was no monotonic dose-response relationship. When a monotonic dose-response relationship existed, the results showed that TEPI was comparable to mTPI.

TEPI displayed a higher probability of stopping early when no efficacious dose level existed (see Scenario #1 in Table 7).

The simulated data in these scenarios demonstrated that the TEPI design for clinical trial dose recommendations simultaneously optimizes toxicity and efficacy, e.g., antitumor activity. This may allow for accelerated development of potent novel agents and/or therapies for treating diseases and disorders, including in oncology. The TEPI design allowed for close collaboration with clinicians in choosing the design parameters to make the dosing decisions sensible while reflecting clinical practice.

Based on simulations, the TEPI design is a good approach for dose escalation studies where one can continuously monitor toxicity and efficacy. The approach is easy to understand and implement.

### Example 4: Clinical Trial Dosage Recommendation Simulations Based on Toxicity and Efficacy Probability Intervals (TEPI)

The TEPI design described in Example 1 was tested in five additional scenarios and simulations were run. For the further scenarios 1 and 2, there were three dose levels and for the further scenarios 3-5 there were four dose levels. For each trial, the performance for both a small sample size (n=12 or 15) and a moderate sample size (n=21 or 27) was assessed. Table 13 summarizes the true toxicity probability (Tox) and true efficacy probability (Response) at each dose level in the assessed scenarios.

In the simulation, the following parameters were set for the TEPI design: the maximum acceptable toxicity rate, p_{T}, was set at 0.35; the minimum acceptable response rate (efficacy), q_{E}, was set at 0.4; α=1; β=1; delta=0.2; η=0.95 and ξ=0.95. Under each scenario and for each sample size, 10,000 trial simulations were conducted in silico. The results are shown in Table 13.

TABLE 13

| | Dose | 1 | 2 | 3 | 4 | |
|---|---|---|---|---|---|---|
| Scenario 1 | Tox | 0.1 | 0.2 | 0.3 | | None |
| | Eff | 0.5 | 0.7 | 0.9 | | |
| n = 21 | % BEST | 17.9 | 50.4 | 31.5 | | 0.2 |
| | # PTS | 5.74 | 9.63 | 5.60 | | |
| n = 12 | % BEST | 29.1 | 46.1 | 24.7 | | 0.1 |
| | # PTS | 4.67 | 5.17 | 2.15 | | |
| Scenario 2 | Tox | 0.1 | 0.2 | 0.3 | | None |
| | Eff | 0.1 | 0.5 | 0.3 | | |
| n = 21 | % BEST | 15.2 | 76.6 | 6.8 | | 1.4 |
| | # PTS | 3.75 | 7.76 | 9.38 | | |
| n = 12 | % BEST | 14.1 | 72.1 | 12.7 | | 1.2 |
| | # PTS | 3.51 | 4.75 | 3.72 | | |
| Scenario 3 | Tox | 0.05 | 0.35 | 0.55 | 0.9 | None |
| | Eff | 0.1 | 0.7 | 0.9 | 0.3 | |
| n = 27 | % BEST | 2.1 | 78.3 | 6.5 | 0 | 13.1 |
| | # PTS | 5.80 | 17.23 | 2.67 | 0.04 | |
| n = 15 | % BEST | 4.6 | 80.5 | 9.24 | 0 | 5.6 |
| | # PTS | 4.73 | 8.31 | 1.75 | 0.03 | |
| Scenario 4 | Tox | 0.1 | 0.2 | 0.3 | 0.7 | None |
| | Eff | 0.1 | 0.7 | 0.2 | 0.1 | |
| n = 27 | % BEST | 3.9 | 94.6 | 0.4 | 0.0 | 1.1 |
| | # PTS | 3.85 | 14.29 | 6.10 | 2.61 | |
| n = 15 | % BEST | 5.2 | 91.2 | 2.4 | 0.2 | 1.0 |
| | # PTS | 3.67 | 7.36 | 2.56 | 1.37 | |
| Scenario 5 | Tox | 0.1 | 0.2 | 0.3 | 0.7 | None |
| | Eff | 0.1 | 0.3 | 0.5 | 0.6 | |
| n = 27 | % BEST | 17.0 | 25.5 | 51.3 | 4.6 | 1.8 |
| | # PTS | 3.85 | 6.93 | 12.50 | 3.55 | |
| n = 15 | % BEST | 14.1 | 32.8 | 46.1 | 5.3 | 1.6 |
| | # PTS | 3.58 | 4.56 | 5.00 | 1.82 | |

### Example 5: Dosage Recommendation Simulations Based on Toxicity and Efficacy Probability Interval (TEPI) Model Compared with EffTox Model

The TEPI design described in Example 1 was tested in 1000 simulation runs and was compared to another approach, EffTox (Thall and Cook, Biometrics (2004) 60: 684-693), that considers both efficacy and toxicity in the dose escalation decision. EffTox is an adaptive Bayesian method and involves modeling the joint probability of efficacy and toxicity outcomes using logistic regression and copula models. With EffTox, doses for successive cohorts of subjects are selected based on a set of efficacy-toxicity trade-off contours. Prior distributions must be chosen prior to the start of the trial for six components of the parameter in the model. To find the contour, clinicians need to specify the minimum acceptable response rate if treatment has no toxicity, the highest tolerable toxicity if the treatment is 100% efficacious, and an intermediate point that depicts a realistic efficacy-toxicity trade-off.

For simulations comparing the EffTox method and the TEPI method, the same scenario setup as in Berry et al. (Chapman & Hall/CRC Biostatistics Series, Vol. 38, Chapter 3, 3.3.4) was used. The following parameters were used: minimum acceptable response rate (efficacy), q_{E}, was set at 0.2; maximum acceptable toxicity rate, p_{T}, was set at 0.5; true probability of efficacy q=(0.1, 0.3, 0.6, 0.7) and true probability of toxicity p=(0.05, 0.15, 0.2, 0.5). For TEPI, Tables 6A and 6B were used for dose-finding and a slightly different efficacy utility function was used for final dose selection in which the efficacy utility, based on the efficacy utility function, increased linearly from 0.2 to 1. This TEPI setup is referred to as scenario 7.

For the simulations, the maximum sample size was 60 subjects and the cohort size was 3 subjects. The simulations assumed dichotomous efficacy and safety outcomes that were independent, with a monotonic relationship between toxicity and dose. 1000 trial simulations were conducted in silico. The results are shown in Table 14.

TABLE 14

| | | | EffTox | | TEPI | |
|---|---|---|---|---|---|---|
| Dose | True Prob | | Sel Prob | | Sel Prob | |
| Level | Tox | Response | (%) | % of N | (%) | % of N |
| 1 | 0.05 | 0.1 | 0.1 | 5.7 | 3.2 | 7.2 |
| 2 | 0.15 | 0.3 | 13.3 | 20 | 15.4 | 10.1 |
| 3 | 0.2 | 0.6 | 75.6 | 56.7 | 76.3 | 43.6 |
| 4 | 0.5 | 0.7 | 11 | 17.7 | 1.7 | 39.1 |

As shown in Table 14, the TEPI design selected the optimal dose 76.3% of the time as compared with the EffTox model that selected the optimal dose 75.6% of the time. Thus, in this scenario, the selection probability of the most desirable dose was almost identical between the two methods, but TEPI allocated a higher percentage of subjects to dose level 4.

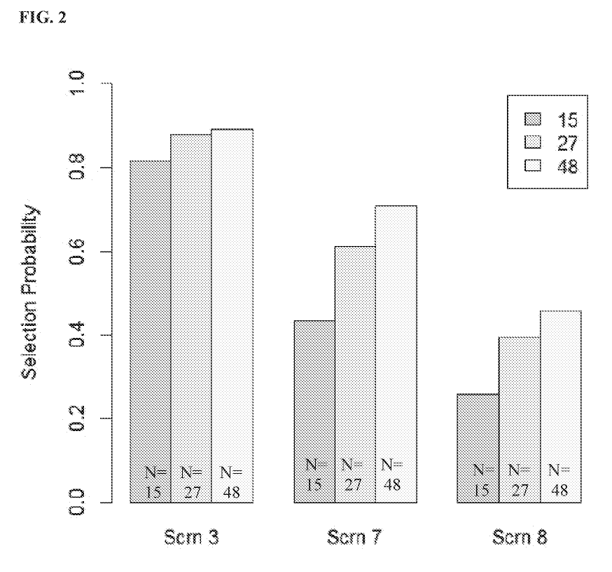

For TEPI, the same simulation set-up was run but with a different sample size of 15, 27, and 48 (see Example 6 below). Even at the smaller sample size, TEPI still selected the optimal dose with 40-70% probability, as shown in FIG. 2. For the EffTox model, a larger sample size was required to produce satisfactory results.

The data demonstrated that the TEPI design for clinical trial dose recommendations simultaneously optimizes toxicity and efficacy, e.g., antitumor activity. This may allow for accelerated development of potent novel agents and/or therapies for treating diseases and disorders, including in oncology.

Based on the simulations, the TEPI design is a good approach for dose escalation studies where one can continuously monitor toxicity and efficacy. The approach is easier to understand and implement than other models, such as the EffTox model.

### Example 6: Sample Size Impacts on Selection Probability

A simulation study was performed to evaluate the impact of varying sample size on the identification of the highest utility dose, e.g., optimal dose, by the TEPI model described in the above examples. The study was performed based on Scenarios 3 described in Example 7 below and scenario 7 described in Example 5. In addition, a further additional scenario (Scenario 8) was assessed with true probabilities of toxicity p=(0.1, 0.2, 0.3, 0.7) and true probability of efficacy q=(0.05, 0.2, 0.5, 0.6). The simulations were performed with a sample size of 15, 27 or 48.

The results for the simulations are shown in FIG. 2, which plots the probability that the true highest utility dose was selected against the sample size of 15, 27, and 48 in the simulated trial. In the scenarios, it was observed that with the increase of sample size, the percentage of time the true highest utility dose was chosen increased. Similar results were observed for other scenarios as well.

This result demonstrates that even under a small sample size of 15 or 27, the TEPI model performed well.

### Example 7: Additional Simulations

An additional simulation study was performed to compare the TEPI design to performance with the 3+3, CRM and mTPI, which are designs that only consider toxicity outcomes. The simulations were based on the following 6 scenarios:
Scenario 1: All doses safe; No dose is efficacious
Scenario 2: All doses are safe with the same efficacy
Scenario 3: All doses are safe with toxicity increasing and efficacy decreasing with dose
Scenario 4: Two higher doses unsafe and all doses efficacious but dose level 2 and 3 with similar efficacy
Scenario 5: All doses are safe but efficacy is reaching plateau
Scenario 6: All doses efficacious but no dose safe

In addition, the simulations also were carried out to compare TEPI with the EffTox model as described in Example 5, which uses both toxicity and efficacy outcomes. For each scenario, the maximum tolerable toxicity probability, p_{T}, was 0.4 and the minimum efficacy probability q_{E} was 0.2. The maximum sample size for the simulations was set at 27 and 1,000 trials were simulated for each scenario.

For TEPI, the simulation was carried out based on the decisions described in Table 4B and Tables 6A and 6B using hyperparameters αₚ=βₚ=α_{q}=β_{q}=1. For mTPI, the equivalence interval was set to [0.25, 0.35], and based on the dose-finding Table set forth as Table 15. For mTPI, 0.35 was used as the upper bound of the equivalence interval instead of 0.4 because it can be difficult to justify such a high toxicity rate without considering efficacy data. NextGen-DF (Yang et al., Contemp Clin Trials (2015) 45(PtB): 426-434) was used to implement the standard 3+3 design, the CRM design as implemented in the R package “dfCRM” (Cheung, Clinical Trials (2013) 10(6): 852-861), and the mTPI design. The simulation scenarios were based on 1,000 simulated trials with a maximum sample size of 27 patients and a cohort size of 3.

TABLE 15

| | | Number of Patients at Current Dose | | | | | | | | |
|---|---|---|---|---|---|---|---|---|---|---|
| | | 3 | 6 | 9 | 12 | 15 | 18 | 21 | 24 | 27 |
| Number of DLTs | 0 | E | E | E | E | E | E | E | E | E |
| | 1 | S | E | E | E | E | E | E | E | E |
| | 2 | D | S | S | E | E | E | E | E | E |
| | 3 | DU_{T} | S | S | S | S | E | E | E | E |
| | 4 | | DU_{T} | S | S | S | S | E | E | E |
| | 5 | | DU_{T} | DU_{T} | S | S | S | S | S | E |
| | 6 | | DU_{T} | DU_{T} | D | S | S | S | S | S |
| | 7 | | | DU_{T} | DU_{T} | S | S | S | S | S |
| | 8 | | | DU_{T} | DU_{T} | DU_{T} | S | S | S | S |
| | 9 | | | DU_{T} | DU_{T} | DU_{T} | DU_{T} | S | S | S |
| | 10 | | | | DU_{T} | DU_{T} | DU_{T} | DU_{T} | S | S |
| | 11 | | | | DU_{T} | DU_{T} | DU_{T} | DU_{T} | DU_{T} | S |
| | 12 | | | | DU_{T} | DU_{T} | DU_{T} | DU_{T} | DU_{T} | DU_{T} |

The results for each of these scenarios 1-6 are depicted in Table 16.

TABLE 16

| | | | True | | | | | | | | | | |
|---|---|---|---|---|---|---|---|---|---|---|---|---|---|
| | Dose | | Prob | Selection Prob (%) | | | | | Number of Subjects Treated | | | | |
| Scenario | Level | Tox | Eff | TEPI | mTPI | 3 + 3 | CRM | EffTox | TEPI | mTPI | 3 + 3 | CRM | EffTox |
| 1 | 1 | 0.16 | 0.05 | 22.1 | 12.1 | 23.8 | 7.9 | 0 | 6.02 | 7.7 | 4.6 | 7.9 | 3.1 |
| | 2 | 0.2 | 0.1 | 17.9 | 24.1 | 22.0 | 23.6 | 0 | 5.7 | 8.1 | 3.8 | 7.5 | 3.1 |
| | 3 | 0.25 | 0.15 | 17.2 | 30.6 | 16.0 | 31.9 | 0 | 5.1 | 6.1 | 2.7 | 6.3 | 3.8 |
| | 4 | 0.3 | 0.18 | 7.5 | 32.1 | 15.8 | 36.6 | 8 | 4.3 | 4.9 | 1.4 | 5.2 | 5.3 |

| | TEPI | mTPI | 3 + 3 | CRM | EffTox |
|---|---|---|---|---|---|
| Probability of | 35.3 | 1.1 | 22.4 | 0.0 | 92 |
| early termination | | | | | |
| Average # of | 21.2 | 26.8 | 12.6 | 27.0 | 15.3 |
| subjects treated | | | | | |
| in a trial | | | | | |

| | | | True | | | | | | | | | | |
|---|---|---|---|---|---|---|---|---|---|---|---|---|---|
| | Dose | | Prob | Selection Prob (%) | | | | | Number of Subjects Treated | | | | |
| | Level | Tox | Eff | TEPI | mTPI | 3 + 3 | CRM | EffTox | TEPI | mTPI | 3 + 3 | CRM | EffTox |
| 2 | 1 | 0.15 | 0.8 | 83.9 | 10.7 | 23.4 | 6.3 | 66 | 9.1 | 7.4 | 4.5 | 7.5 | 17.8 |
| | 2 | 0.2 | 0.8 | 13.6 | 25.8 | 22.6 | 23.6 | 30 | 8.5 | 8.3 | 3.9 | 7.6 | 8.4 |
| | 3 | 0.25 | 0.8 | 2.1 | 28.2 | 17.1 | 32.7 | 3 | 5.6 | 6.0 | 2.9 | 6.5 | 0.7 |
| | 4 | 0.3 | 0.8 | 0.3 | 34.6 | 16.1 | 37.4 | 1 | 3.8 | 5.1 | 1.5 | 5.3 | 0.1 |

| | TEPI | mTPI | 3 + 3 | CRM | EffTox |
|---|---|---|---|---|---|
| Probability of | 0.1 | 0.0 | 20.8 | 0.0 | 0 |
| early termination | | | | | |
| Average # of | 27.0 | 27.0 | 12.8 | 27.0 | 27.0 |
| subjects treated | | | | | |
| in a trial | | | | | |

| | | | True | | | | | | | | | | |
|---|---|---|---|---|---|---|---|---|---|---|---|---|---|
| | Dose | | Prob | Selection Prob (%) | | | | | Number of Subjects Treated | | | | |
| Scenario | Level | Tox | Eff | TEPI | mTPI | 3 + 3 | CRM | EffTox | TEPI | mTPI | 3 + 3 | CRM | EffTox |
| 3 | 1 | 0.1 | 0.1 | 7.2 | 4.0 | 25.8 | 2.1 | 3 | 4.4 | 5.4 | 4.4 | 5.5 | 3.7 |
| | 2 | 0.2 | 0.7 | 88.0 | 32.5 | 36.4 | 32.1 | 42 | 12.3 | 9.8 | 4.6 | 8.9 | 11.8 |
| | 3 | 0.3 | 0.2 | 0.3 | 60.7 | 25.9 | 60.6 | 3 | 7.1 | 9.8 | 3.5 | 10.0 | 4.3 |
| | 4 | 0.7 | 0.1 | 0.1 | 2.5 | 1.4 | 5.2 | 2 | 2.3 | 1.9 | 1.1 | 2.6 | 2.0 |

| | TEPI | mTPI | 3 + 3 | CRM | EffTox |
|---|---|---|---|---|---|
| Probability of | 4.4 | 0.3 | 10.5 | 0.0 | 50 |
| early termination | | | | | |
| Average # of | 26.1 | 26.9 | 13.6 | 27.0 | 21.8 |
| subjects treated | | | | | |
| in a trial | | | | | |

| | | | True | | | | | | | | | | |
|---|---|---|---|---|---|---|---|---|---|---|---|---|---|
| | Dose | | Prob | Selection Prob (%) | | | | | Number of Subjects Treated | | | | |
| | Level | Tox | Eff | TEPI | mTPI | 3 + 3 | CRM | EffTox | TEPI | mTPI | 3 + 3 | CRM | EffTox |
| 4 | 1 | 0.15 | 0.43 | 53.9 | 11.3 | 23.8 | 7.7 | 19 | 6.1 | 7.3 | 4.6 | 7.7 | 7.3 |
| | 2 | 0.2 | 0.52 | 41.3 | 41.6 | 41.0 | 43.8 | 49 | 9.6 | 9.6 | 4.3 | 9.7 | 12.4 |
| | 3 | 0.4 | 0.5 | 3.6 | 38.8 | 11.7 | 41.7 | 22 | 9.0 | 7.9 | 2.8 | 7.6 | 5.3 |
| | 4 | 0.5 | 0.6 | 1.2 | 7.1 | 2.5 | 6.8 | 5 | 2.1 | 1.9 | 0.7 | 2.0 | 1.1 |

| | TEPI | mTPI | 3 + 3 | CRM | EffTox |
|---|---|---|---|---|---|
| Probability of | 1.2 | 1.2 | 21.0 | 0.0 | 6 |
| early termination | | | | | |
| Average # of | 27.0 | 27.0 | 12.4 | 27.0 | 26.1 |
| subjects treated | | | | | |
| in a trial | | | | | |

| | | | True | | | | | | | | | | |
|---|---|---|---|---|---|---|---|---|---|---|---|---|---|
| | Dose | | Prob | Selection Prob (%) | | | | | Number of Subjects Treated | | | | |
| Scenario | Level | Tox | Eff | TEPI | mTPI | 3 + 3 | CRM | EffTox | TEPI | mTPI | 3 + 3 | CRM | EffTox |
| 5 | 1 | 0.1 | 0.2 | 16.4 | 5.1 | 27.0 | 3.1 | 1 | 4.7 | 5.6 | 4.4 | 5.4 | 3.6 |
| | 2 | 0.2 | 0.6 | 65.4 | 31.5 | 34.8 | 25.0 | 48 | 8.6 | 9.6 | 4.6 | 8.2 | 12.4 |
| | 3 | 0.3 | 0.6 | 13.8 | 44.4 | 19.5 | 45.4 | 38 | 7.2 | 8.1 | 3.4 | 8.6 | 8.5 |
| | 4 | 0.4 | 0.6 | 1.0 | 19.0 | 9.1 | 26.5 | 10 | 4.9 | 3.7 | 1.4 | 4.8 | 2.0 |

| | TEPI | mTPI | 3 + 3 | CRM | EffTox |
|---|---|---|---|---|---|
| Probability of | 3.4 | 0.0 | 9.6 | 0.0 | 3 |
| early termination | | | | | |
| Average # of | 26.3 | 27.0 | 13.8 | 27.0 | 26.5 |
| subjects treated | | | | | |
| in a trial | | | | | |

| | | | True | | | | | | | | | | |
|---|---|---|---|---|---|---|---|---|---|---|---|---|---|
| | Dose | | Prob | Selection Prob (%) | | | | | Number of Subjects Treated | | | | |
| | Level | Tox | Eff | TEPI | mTPI | 3 + 3 | CRM | EffTox | TEPI | mTPI | 3 + 3 | CRM | EffTox |
| 6 | 1 | 0.5 | 0.4 | 33.9 | 23.0 | 10.7 | 99.3 | 16 | 14.9 | 14.0 | 4.5 | 25.8 | 7.9 |
| | 2 | 0.6 | 0.5 | 0.3 | 0.9 | 0.3 | 0.7 | 13 | 1.8 | 1.2 | 0.6 | 1.1 | 6.6 |
| | 3 | 0.7 | 0.6 | 0.0 | 0.1 | 0.2 | 0.0 | 2 | 0.1 | 0.1 | 0.0 | 0.1 | 1.2 |
| | 4 | 0.8 | 0.8 | 0.0 | 0.0 | 0.0 | 0.0 | 0 | 0.0 | 0.0 | 0.0 | 0.0 | 0.2 |

| | TEPI | mTPI | 3 + 3 | CRM | EffTox |
|---|---|---|---|---|---|
| Probability of | 65.8 | 76.0 | 88.8 | 0.0 | 69 |
| early termination | | | | | |
| Average # of | 16.8 | 15.3 | 5.2 | 27.0 | 15.9 |
| subjects treated | | | | | |
| in a trial | | | | | |

In scenario 1, all four doses were tolerable but unacceptable due to low response rates. With the TEPI design, 35% of the trials terminated early with an average sample size of 21. As expected, the mTPI and CRM designs almost never stopped early and exhausted the sample size, while the 3+3 design stopped early 22% of the time. Thus, TEPI more effectively terminated the trials early, minimizing the number of patients treated with a drug (or the selected dose ranges of a drug) having minimal antitumor activity. In this scenario, TEPI allocated similar numbers of patients across doses.

Scenario 2 was an extreme case where all doses were safe but had the same high response rate, so the starting dose had the highest utility. TEPI selected this dose level 84% of the time compared to 6-24% by other designs, and TEPI never selected dose levels 3 or 4. TEPI put more patients on average at dose levels 1 and 2, and fewer patients at the dose levels 3 and 4 than the mTPI and CRM designs.

Under scenario 3, dose levels 1-3 were safe, dose level 4 was unsafe, and dose level 2 had the highest efficacy. TEPI selected dose level 2 with a probability of 88% and allocated an average of 12 patients to this dose level. In contrast, mTPI and CRM selected this dose level with much smaller probabilities and allocated fewer patients thereto. Moreover, mTPI and CRM selected dose level 3 with 60% probability, while TEPI only selected this level 0.3% of the time. A similar trend was observed for dose level 4.

In scenario 4, the two lower doses were safe and effective, while the two higher doses were unsafe. There was a monotonic increasing relationship between response and dose, which was the underlying assumption for the mTPI, 3+3 and CRM designs. In this scenario, dose level 2 had the highest utility. The four designs selected this dose with similar probability (˜40%), which demonstrated that TEPI has good performance characteristics even under the conventional assumptions. However, TEPI had a 54% probability of selecting dose level 1, which had slightly lower utility than dose level 2. Both mTPI and CRM were aggressive, selecting the unsafe dose (level 3 or 4), 46% and 49% of the time, respectively; in comparison, the 3+3 design selected an unsafe dose 14.2% of the time, and TEPI only 5% of the time.

In scenario 5, all doses were tolerable and efficacy increased from dose level 1 but plateaued at dose level 2. Dose level 2 was optimal. TEPI selected this optimal dose level with a probability of 65% and allocated an average 8.6 patients at this dose level. In contrast, mTPI and CRM selected this dose with 31% and 25% probabilities, respectively, while both selected the suboptimal dose level 3 with approximately 45% probability.

In scenario 6, all doses were too toxic despite having acceptable efficacy. In this case, TEPI terminated early 66% of the times with an average sample size of 17, and mTPI terminated early 76% of the times with an average sample size of 15. CRM did not stop early with an average of 26 patients treated at dose levels 1 and 2 compared to 15 and 14 patients treated at this dose by the TEPI and mTPI models. Here, TEPI seemed more aggressive than mTPI, which was expected because dose level 1 had acceptable efficacy.

In all the scenarios where an acceptable dose existed (e.g., scenarios 2, 3, 4, 5), the simulations showed that the 3+3 was less likely to select the desirable doses compared to the TEPI model. It appeared that the 3+3 design was too conservative in that it was unable to escalate quickly even when the doses were safe.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1-86. (canceled)

87. A method of dosing a subject with a therapeutic agent for treating a disease or condition, comprising:
administering a therapeutic agent to a subject that has a disease or condition, wherein the therapeutic agent is administered at a dose level according to a selected dose recommendation, wherein said dose recommendation is selected from instructions produced by:
i) obtaining a matrix by designating two or more toxicity probability intervals and two or more efficacy probability intervals of the therapeutic agent, respectively, wherein a dosing action is assigned to each combination of toxicity and efficacy probability intervals;
ii) determining a joint unit probability mass (UPM) for each combination of toxicity and efficacy probability intervals based on one or more toxicity probabilities at current dose level i (pᵢ) and one or more efficacy probabilities at current dose level i (qᵢ);
iii) identifying the combination of toxicity and efficacy intervals that has the highest joint UPM; and
iv) assigning the dosing action associated with the identified combination as a dose recommendation, thereby producing the instructions.

88. A method of dosing a subject with a therapeutic agent for treating a disease or condition, comprising:
a) obtaining instructions that specify a dose recommendation, wherein the instructions are produced by:
i) obtaining a matrix by designating two or more toxicity probability intervals and two or more efficacy probability intervals of the therapeutic agent, respectively, wherein a dosing action to each combination of toxicity and efficacy probability intervals;
ii) determining a joint unit probability mass (UPM) for each combination of toxicity and efficacy probability intervals based on one or more possible toxicity probabilities at current dose level i (pᵢ) and one or more possible efficacy probabilities at current dose level i (qᵢ);
iv) identifying the combination of toxicity and efficacy intervals that has the highest joint UPM; and
v) assigning the dosing action associated with the identified combination as a dose recommendation, thereby producing the instructions;
b) administering the therapeutic agent to the subject at a dose level according to the instructions.

89. The method of claim 87, wherein the matrix comprises three or more toxicity probability intervals.

90. The method of claim 87, wherein the matrix comprises three or more efficacy probability intervals.

91. The method of claim 87, wherein at least one dosing action or dose recommendation is:
(a) escalate (E) to dose level i+1;
(b) stay (S) at dose level i; or
(c) de-escalate (D) to dose level i−1.

92. The method of claim 87, comprising prior to step i) obtaining the maximum acceptable toxicity probability (p_{T}) and minimum acceptable efficacy probability (q_{E}) of the therapeutic agent.

93. The method of claim 92, further comprising prior to producing the instructions, altering the dose recommendation to:
a) de-escalate and not return to current dose if the probability that pᵢ is greater than the maximum acceptable toxicity probability (p_{T}) exceeds 0.95;
b) de-escalate and not return to current dose if the probability that qᵢ is less than the minimum acceptable efficacy probability (q_{E}) exceeds 0.7; or
c) escalate and not return to current dose if the probability that qᵢ is less than q_{E} exceeds 0.7.

94. The method of claim 92, wherein prior to producing instructions, the method further comprises altering the dose recommendation to:
(a) de-escalate and not return to current dose if pᵢ is greater than p_{T};
(b) de-escalate and not return to current dose if qᵢ is less than q_{E}; or
(c) escalate and not return to current dose if qᵢ is less than q_{E}.

95. The method of claim 87, wherein at least one dose action or dose recommendation is:
(a) de-escalate and do not return to the current dose level or any higher dose level (DU);
(b) de-escalate and do not return to the current dose level or any higher dose level (DEU);
or
(c) escalate and do not return to the current dose level or any lower dose level (EEU).

96. The method of claim 87, wherein each toxicity probability interval is defined by a start value a and an end value b and each efficacy probability interval is defined by a start value c and an end value d.

97. The method of claim 96, wherein each combination of toxicity and efficacy probability intervals is defined as (a, b)×(c, d).

98. The method of claim 87, wherein the matrix comprises a two-way grid, and wherein the dosing actions associated with the combination of toxicity and efficacy probability intervals are displayed in the two-way grid.

99. The method of claim 87, wherein determining the joint UPM for each combination of toxicity and probability intervals comprises:
a) determining the probability that pᵢ and qᵢ are contained within the combination of toxicity and probability intervals;
b) dividing the probability determined in step a) by the product of the toxicity probability interval length and the efficacy probability interval length.

100. The method of claim 97, wherein the joint UPM (JUPM) is determined as:
$$JUPM \begin{matrix}( c , d ) \\ ( a , b )\end{matrix} ≡ \frac{Pr ( {p}_{i} ∈ ( a , b ) , {q}_{i} ∈ ( c , d ) \vert 𝒟 )}{( b - a ) \times ( d - c )} , \\ 0 < a < b < 1 , 0 < c < d < 1. \qquad ( 1 )$$

101. The method of claim 87, wherein determining the joint UPM is based on the posterior distributions of pᵢ and qᵢ according to Bayes' rule.

102. The method of claim 87, wherein the toxicity and efficacy probability are associated with a rate of a toxic outcome or a rate of a response outcome, respectively.

103. The method of claim 87, wherein pᵢ is a ratio of a number of subjects experiencing a toxic outcome (xᵢ) to a total number of subjects (nᵢ) administered with the therapeutic agent at dose level i.

104. The method of claim 87, wherein qi is a ratio of the number of subjects experiencing a response outcome (yᵢ) to a total number of subjects (nᵢ) administered with the therapeutic agent at dose level i.

105. The method of claim 102, wherein the toxicity outcome is a dose-limiting toxicity (DLT).

106. The method of claim 102, wherein the response outcome is a complete response (CR).

107. The method of claim 87, wherein the instructions comprise one or more decision tables.

108. The method of claim 103, wherein nᵢ is within a range from about 1 to about 100.

109. The method of claim 87, wherein one or more of steps of obtaining a matrix, determining a joint unit probability mass (UPM), identifying the combination with the highest joint UPM, assigning the dosing action associated with the identified combination, and producing or outputting instructions occur at an electronic device comprising one or more processors and memory.

110. The method of claim 87, wherein the therapeutic agent is administered for a clinical trial.

111. The method of claim 110, wherein the selected dose is determined based on the number of subjects previously treated in the clinical trial and the actual probabilities of the toxic outcomes and response outcomes among the subjects previously treated.

112. The method of claim 110, wherein the clinical trial is terminated when the number of subjects enrolled reaches a pre-specified maximum.

113. The method of claim 112, wherein the pre-specified maximum is within a range from about 1 to about 100.

114. The method of claim 92, further comprising identifying an optimal dose level, wherein the optimal dose level is associated with the highest probability that pᵢ is less than p_{T} and qᵢ is less than q_{E}.

115. The method of claim 87, further comprising identifying an optimal dose level, wherein the optimal dose level is identified based on a combined utility function determined from a safety utility function ƒ₁(p) and an efficacy utility function ƒ₂(q), wherein p is associated with a rate of a toxic outcome, and q is associated with a rate of a response outcome, respectively.

116. The method of claim 115, wherein the combined utility function U(p,q) is determined as:
U(p,q)=ƒ₁(p)ƒ₂(q)

117. The method of claim 115, further comprising determining a posterior expected utility for each dose level, wherein the optimal dose level is determined based on the posterior utility.

118. The method of claim 117, wherein the posterior utility is determined as:
$$\hat{E} [ U ( {p}_{i} , {q}_{i} ) \vert D ] = \frac{1}{T} {\sum }_{t = 1}^{T} {U}^{t} ( {\hat{p}}_{i}^{t} , {q}_{i}^{t} ) .$$

119. The method of claim 118, wherein the optimal dose level is associated with the largest posterior utility.

120. The method of claim 119, wherein the optimal dose level is determined by:
î=argmaxᵢE[U(pᵢ,qᵢ)| [symbol] ]

121. The method of claim 87, wherein the disease or condition is a tumor or a cancer.

122. The method of claim 87, wherein the therapeutic agent is one for which the response outcome can be assessed within the timeframe in which the toxicity outcome is assessed.

123. The method of claim 87, wherein the therapeutic agent is or comprises a small molecule, a gene therapy, a transplant or an adoptive cell therapy.

124. The method of claim 123, wherein the therapeutic agent is or comprises an adoptive cell therapy.

125. The method of claim 124, wherein the adoptive cell therapy comprises cells expressing a chimeric antigen receptor (CAR).

126. The method of claim 124, wherein the cells are administered at a dose level that is between about 0.5×10⁶ cells/kg body weight of the subject and 6×10⁶ cells/kg.

127. The method of claim 124, wherein the number of cells administered is between about 1×10⁶ and about 1×10⁸ CAR expressing cells.

128. The method of claim 124, wherein the dose of cells are administered in a single pharmaceutical composition comprising the cells of the dose.

129. The method of claim 124, wherein:
the dose is a split dose, wherein the cells of the dose are administered in a plurality of compositions, collectively comprising the cells of the dose, which, optionally, are administered over a period of no more than three days.

130. The method of claim 121, wherein the disease or condition is a leukemia or lymphoma.

131. The method of claim 130, wherein the disease or condition is acute lymphoblastic leukemia or non-Hodgkin lymphoma (NHL).

132. A method for providing a dose recommendation for a therapeutic agent, comprising:
a) obtaining a matrix comprising one or more dosing actions associated with a combination of toxicity and efficacy probability intervals;
b) determining a joint unit probability mass (UPM) for each combination of toxicity and efficacy probability intervals for one or more possible toxicity probabilities at current dose level i (pᵢ) and one or more possible efficacy probabilities at current dose level i (qᵢ);
c) identifying the combination of toxicity and efficacy intervals that has the highest joint UPM;
d) assigning the dosing action associated with the identified combination as a dose recommendation for each of the one or more possible toxicity and efficacy probabilities; and
e) producing or outputting instructions that specify the dose recommendations.

133. A computer implemented method for providing a dose recommendation for a therapeutic agent, comprising:
at an electronic device having a processor and memory:
a) obtaining a matrix comprising one or more dosing actions associated with a combination of toxicity and efficacy probability intervals;
b) determining, by the processor, a joint unit probability mass (UPM) for each combination of toxicity and efficacy probability intervals for one or more possible toxicity probabilities at current dose level i (pᵢ) and one or more possible efficacy probabilities at current dose level i (qᵢ);
c) identifying the combination of toxicity and efficacy intervals that has the highest joint UPM;
d) assigning the dosing action associated with the identified combination as a dose recommendation for each of the one or more possible toxicity and efficacy probabilities; and
e) producing or outputting instructions that specify the dose recommendations.

134. A computer system comprising a processor and memory, the memory comprising instructions operable to cause the processor to carry out one or more of steps of the method of claim 87, wherein the steps are selected from obtaining a matrix, determining a joint unit probability mass (UPM) for each combination of toxicity and efficacy probability intervals, identifying the combination with the highest joint UPM, assigning the dosing action associated with the identified combination, and producing instructions.

135. A computer system comprising a processor and memory, the memory comprising instructions operable to cause the processor to carry out one or more of steps of the method of claim 87.

136. Dose recommendation instructions for performing a clinical trial produced by the method of claim 87.
